SEQUENCE LISTING (4 sequences)

SEQ ID NO: 1 - protein, 289 aa, Synthetic construct
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEVDGGGGGLTDTLQAETDQLEDEKSALQTEIANLLKEKEKLEFILAAGGSGGSGLNDIFEAQKIEWHEHHHHHH

SEQ ID NO: 2 - protein, 289 aa, Synthetic construct; identical to SEQ ID NO: 1

SEQ ID NO: 3 - protein, 310 aa, Synthetic construct
MVCLKLPGGSCMTALTVTLMVLSSPLALAGDTGLPVSKMRMATPLLMQASGGGSLVPRGSGGGGSGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKVDGGGGGRIARLEEKVKTLKAQNSELASTANMLREQVAQLKQKVMNH

SEQ ID NO: 4 - protein, 601 aa, Synthetic construct
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSESPFGNEKLTFGTGTRLTIIPIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRRKRGSGATNFSLLKQAGDVEENPGPMGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSSTGLPYGYTFGSGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

(19) United States
(12) Patent Application Publication
(10) Pub. No.: US 2026/0130992 A1
(43) Pub. Date: May 14, 2026

(54) NANOPARTICLES FOR DELIVERY OF IMMUNOREGULATORY MATERIALS TO T CELLS

(71) Applicant: The Johns Hopkins University, Baltimore, MD (US)

(72) Inventors: Jonathan Schneck, Silver Spring, MD (US); Ariel Isser, Baltimore, MD (US)

(21) Appl. No.: 19/118,262

(22) PCT Filed: Oct. 4, 2023
(86) PCT No.: PCT/US2023/075959

§ 371 Date: Apr. 3, 2025

Related U.S. Application Data
(60) Provisional application No. 63/378,441, filed on Oct. 5, 2022.

(51) Int. Cl. A61K 40/11; A61K 35/17; A61K 40/24; A61K 40/33; A61K 40/42; A61P 35/00; C12N 5/0783

(52) U.S. Cl. CPC A61K 40/11; A61K 35/17; A61K 40/24; A61K 40/33; A61K 40/4213; A61K 40/4224; A61P 35/00; C12N 5/0638; A61K 2239/27; C12N 2501/2302; C12N 2501/2312; C12N 2501/24

(57) ABSTRACT

Artificial antigen presenting cells (aAPC) including a major histocompatibility class II (MHC II) molecule and methods of their use for identifying, isolating, or detecting one or more antigen-specific T cells, and treating a disease, disorder, or condition, including cancer, are disclosed.

### STATEMENT OF GOVERNMENTAL INTEREST

This invention was made with government support under EB028239 awarded by the National Institutes of Health. The government has certain rights in the invention.

### SEQUENCE LISTING

The text of the computer readable sequence listing filed herewith, titled “JHU_41362_601_SequenceListing”, created Oct. 4, 2023, having a file size of 5,987 bytes, is hereby incorporated by reference in its entirety.

### BACKGROUND

Clinical successes of adoptive cell transfer (ACT) therapies across a wide range of hematologic malignancies, Porter et al., 2011; Grupp et al., 2013, and solid tumors, Tran et al., 2014; Hunder et al., 2008, have propelled T cell therapies to the forefront of treatment options for a variety of diseases, especially cancer. Despite their promise, some of the largest hurdles these therapies face in moving toward widespread translation are the associated time, costs, and complexities of ex vivo T cell culture, Isser et al., 2021, as well as the variability of clinical products.

A range of approaches has been developed for ex vivo expansion of tumor-specific T cells, including polyclonal T cell stimulation with plate- or bead-bound anti-CD3 (αCD3) antibodies or antigen-specific T cell stimulation with peptide-pulsed autologous antigen presenting cells (APCs). To simultaneously address the lack of specificity of αCD3 stimulation, as well as the manufacturing challenges and variability of donor-derived APCs, biomimetic artificial APCs (aAPCs) that include MHC proteins and co-stimulatory molecules have been produced. Oelke et al., 2003. Thus far, these synthetic platforms have focused almost exclusively on CD8⁺ T cells, whereas little progress has been made for CD4⁺ targeted technologies.

CD4⁺ T cells serve several critical functions in the antitumor immune response, including recognizing neoantigens that result from tumor-specific mutations, Tran et al., 2014; Kreiter et al., 2015; Alspack et al., 2019; Sahin et al., 2017, recruiting and activating innate immune cells, Mumberg et al., 1999; Hung et al., 1998; Perez-diez et al., 2016; Isser and Schneck, 2018. directly lysing MHC II positive tumor cells, Quezada et al., 2010, and relaying indispensable “help” signals to CD8⁺ T cells to enhance their antitumor function and memory formation. Borst et al., 2018.

### SUMMARY

In some aspects, the presently disclosed subject matter provides an artificial antigen presenting cell (aAPC) comprising a particle having a major histocompatibility complex class II (MHC II) molecule conjugated to a surface thereof. In other aspects, the presently disclosed subject matter provides an artificial antigen presenting cell (aAPC) consisting essentially of a particle having a major histocompatibility complex class II (MHC II) molecule conjugated to a surface thereof.

In certain aspects, the MHC II molecule comprises an MHC II I-A^{b} monomer.

In certain aspects, the aAPC further comprises a costimulatory ligand conjugated to a surface thereof. In particular aspects, the costimulatory ligand is selected from the group consisting of an antibody or antigen-binding fragment thereof that specifically binds to CD28, CD80 (B7-1), CD86 (B7-2), B7- H3, 4-1BBL, 4-1BB, CD27, CD30, CD134 (OX-40L), B7h (B7RP-1), CD40, LIGHT, an antibody or antigen-binding fragment thereof that specifically binds to HVEM, an antibody or antigen-binding fragment thereof that specifically binds to CD40L, an antibody or antigen binding fragment thereof that specifically binds to OX40, and an antibody or antigen-binding fragment thereof that specifically binds to 4-1BB. In more particular aspects, the costimulatory ligand comprises an anti-CD28 (αCD28) antibody.

In some aspects, the aAPC further comprises a major histocompatibility complex class I molecule conjugated to a surface thereof. In certain aspects, the MHC-class I molecule comprises a K^{b}-Ig dimer.

In certain aspects, the MHC II molecule comprises a human leukocyte antigen (HLA) class II monomer. In certain aspects, the HLA class II monomer is selected from the group consisting of HLA-DR, HLA-DP, and HLA-DQ. In certain aspects, the HLA class II monomer comprises DR1 or DR4. In particular aspects, the HLA class II monomer comprises a cleavable thrombin linker, wherein the cleavable thrombin linker enables peptide exchange.

In certain aspects, the HLA class II monomer comprises DR1 fused to an Fc domain. In certain aspects, the Fc domain comprises an amino acid sequence comprising a cysteine substitution. In certain aspects, the Fc domain comprises a cysteine at position 473.

In certain aspects, the HLA class II monomer comprises DR4 fused to an Fc domain. In certain aspects, the Fc domain comprises an amino acid sequence comprising a cysteine substitution. In certain aspects, the Fc domain comprises a cysteine at position 473.

In some aspects, the presently disclosed subject matter provides an aAPC having an HLA class I molecule and an HLA class II molecule conjugated to a surface thereof. In certain aspects, the aAPC comprises DR1 or DR4 monomers with HLA A2-Ig dimers. In certain aspects, the HLA class II monomer comprises DR1 fused to an Fc domain. In certain aspects, the Fc domain comprises an amino acid sequence comprising a cysteine substitution. In certain aspects, the Fc domain comprises a cysteine at position 473. In certain aspects, the HLA class II monomer comprises DR4 fused to an Fc domain. In certain aspects, the Fc domain comprises an amino acid sequence comprising a cysteine substitution. In certain aspects, the Fc domain comprises a cysteine at position 473.

In some aspects, the particle comprises a paramagnetic particle. In particular aspects, the particle comprises an iron-dextran particle.

In other aspects, the presently disclosed subject matter provides a method for identifying, isolating, or detecting one or more antigen-specific T cells, the method comprising:
(a) contacting a plurality of unpurified immune cells comprising one or more antigen-specific T cells with a plurality of aAPCs of any one of claims 1-27;
(b) separating antigen-specific T cells associated with the plurality of aAPCs from cells not associated with the plurality of aAPCs;
(c) recovering antigen-specific T cells associated with the plurality of aAPCs; and
(d) expanding the recovered antigen-specific T cells in culture for a period of time to provide a composition comprising antigen-specific T cells.

In some aspects, the plurality of unpurified immune cells comprising one or more antigen-specific T cells are obtained from a sample comprising one or more of a peripheral blood mononuclear cell (PBMC) sample, memory T cells, naive T cells, previously activated T cells, and tumor infiltrating lymphocytes. In some aspects, the plurality of unpurified immune cells comprising one or more antigen-specific T cells are obtained from a sample comprising one or more of bone marrow, lymph node tissue, spleen tissue, and a tumor.

In certain aspects, the plurality of unpurified immune cells are obtained from a patient or a donor. In certain aspects, the donor comprises a donor who is HLA-matched to an adoptive transfer recipient. In certain aspects, the plurality of unpurified immune cells are obtained from a patient and the patient has one or more diseases, disorders, or conditions selected from the group consisting of a cancer, an infectious disease, and an autoimmune disease.

In some aspects, the one or more antigen-specific T cells are selected from the group consisting of cytotoxic CD4⁺ T cells, CD4⁺ helper T cells, CD8⁺ cytotoxic T lymphocytes, T-helper 17 (Th17) cells, regulatory T cells (Tregs), and combinations thereof.

In certain aspects, the method further comprises ex vivo generation of cytotoxic CD4⁺ T cells.

In some aspects, the method further comprises administering a soluble costimulatory ligand to the antigen-specific T cells associated with the plurality of aAPCs after step (b).

In certain aspects, the method further comprises administering one or more cytokines to the plurality of unpurified immune cells comprising one or more antigen-specific T cells. In particular aspects, the one or more cytokines include one or more of IL-2, IL-12p70, and IFN-γ.

In some aspects, the aAPC comprises a particle having a major histocompatibility complex class II (MHC II) molecule and major histocompatibility complex class I molecule conjugated to a surface thereof. In certain aspects, the method co-activates CD4⁺ and CD8+ T cells. In certain aspects, the co-activation of CD4⁺ and CD8⁺ T cells enhances the therapeutic function and memory formation of the CD8⁺ T cells.

In some aspects, the method comprises redirecting CD4⁺ T cell help of one specificity toward CD8⁺ T cells of a multitude of specificities,

In other aspects, the presently disclosed subject matter provides a method for treating a disease, disorder, or condition, the method comprising administering to a subject in need of treatment thereof a composition comprising one or more antigen-specific T cells prepared by the presently disclosed methods. In certain aspects, the disease, disorder, or condition is selected from the group consisting of a cancer, an infectious disease, and an autoimmune disease. In particular aspects, the disease, disorder, or condition is a cancer and the one or more antigen-specific T cells comprise cytotoxic T cells specific for one or more tumor-associated peptide antigens to the subject in need of treatment thereof. In yet more particular aspects, the cancer comprises a solid tumor or a hematological malignancy. In even yet more particular aspects, the cancer is selected from the group consisting of a melanoma, colon cancer, duodenal cancer, prostate cancer, breast cancer, ovarian cancer, ductal cancer, hepatic cancer, pancreatic cancer, renal cancer, endometrial cancer, testicular cancer, stomach cancer, dysplastic oral mucosa, polyposis, head and neck cancer, invasive oral cancer, non-small cell lung carcinoma, small-cell lung cancer, mesothelioma, transitional and squamous cell urinary carcinoma, brain cancer, a neuroblastoma, and a glioma.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, FIG. 1D, FIG. 1E, FIG. 1F, FIG. 1G, and FIG. 1H demonstrate that MHC II aAPCs stimulate functional antigen-specific murine CD4⁺ T cells. (FIG. 1A) Design of MHC II aAPCs with MHC class II molecules (MHC II) as Signal 1 (S1) and anti-CD28 antibodies (αCD28) as Signal 2 (S2). S2 is either attached to aAPCs (S1/2) or delivered solubly (S1+S2). Created with BioRender.com. (FIG. 1B) Fluorescent quantification of I-A_{OVA}^{b} and αCD28 conjugated to S1/2 and S 1 aAPCs. (FIG. 1C) OT-II CD4⁺ T cell fold proliferation after 7 days of stimulation with I-A_{OVA}^{b} S1/2 aAPCs compared to polyclonal αCD3/αCD28 or I-A^{CLIP}_{b} aAPCs. (FIG. 1D) Day 7 OT-II fold proliferation following treatment with S1 aAPCs and a titration of S2, compared to S1/2 or αCD3/αCD28 aAPCs. (FIG. 1E) Day 7 T-bet staining, (FIG. 1F) CD4⁺ lineage transcription factor staining, and (FIG. 1G) cytokine production of OT-II cells stimulated with S1/2 aAPCs in media containing: no cytokines, IL-2, T cell growth factor (TF) cytokine cocktail, or a Th1 mix (IL-2, IL-12p70, IFN-γ). (FIG. 1H) Day 7 cytokine production of OT-II cells stimulated with S1/2, S1+S2, or αCD3/αCD28 aAPCs versus peptide pulsed OT-II splenocytes or bone-marrow derived dendritic cells (BMDCs). Data in (FIG. 1B-FIG. 1D, FIG. 1F-FIG. 1H) represent mean±standard error of the mean (s.e.m.) from three or more independent experiments. (FIG. 1B) n=8, (FIG. 1C-FIG. 1D) n=4 mice, (FIG. 1F) n=3 (no cytokines) or 4 mice (naive OT-II, IL-2, TF, Th1 mix), (FIG. 1G) n=4 mice, (FIG. 1H) n=4 (S1/2), 5 (BMDCs), or 6 mice (Naive, Spleen APCs, αCD3/αCD28, S1+S2), analyzed using (FIG. 1B) n unpaired Student's t test, two-tailed, (FIG. 1C-FIG. 1D) a one-way ANOVA compared to ‘no stim.’ with Dunnet's multiple-comparisons test, or (FIG. 1F-FIG. 1H) a two-way ANOVA with Tukey's multiple-comparisons test;

FIG. 2A, FIG. 2B, FIG. 2C, FIG. 2D, FIG. 2E, FIG. 2F, FIG. 2G, and FIG. 2H demonstrate that MHC II aAPCs expand rare murine CD4⁺ T cell subsets. (FIG. 2A) Schematic of magnetic enrichment of rare CD4⁺ T cells with aAPCs. Created with BioRender.com. (FIG. 2B) Representative flow plots (left) and fold enrichment (right) of OT-II cells diluted into a B6 background at a ratio of 1:1000 after magnetic enrichment with S1/2 or S1 aAPCs. (FIG. 2C) Representative flow plots and (FIG. 2D) percent of OT-II (cognate) and B6 (non-cognate) CD4⁺ T cells bound to particles after 2 hours of incubation at 37° C. with S1/2 versus S1 aAPCs across a range of doses. (FIG. 2E) Representative flow plots and (FIG. 2F) fold expansion of OT-II and SMART-A1 cells diluted 1:1000 into a B6 background, as measured 7 days after S1+S2 enrichment and expansion. (FIG. 2G) pMHC Tetramer staining and (FIG. 2H) quantified number of I-A_{OVA}^{b} CD4⁺ T cells 7 days after S1/2 or S1+S2 enrichment and expansion. Data in (FIG. 2B, FIG. 2D, FIG. 2F, FIG. 2H) represent mean±standard error of the mean (s.e.m.) from three or more independent experiments. (FIG. 2B) n=4 mice, (FIG. 2D) n=3 (S1/2) or 4 (S1) mice, (FIG. 2F.1) n=6 mice, (FIG. 2F.2) n=3 mice, (FIG. 2H) n=3 (S1/2) or 4 (S1+S2) mice, analyzed using an (FIG. 2B, FIG. 2F, FIG. 2H) unpaired Student's t test, two-tailed or (FIG. 2D) a two-way ANOVA with Tukcy's multiple-comparisons test;

FIG. 3A, FIG. 3B, FIG. 3C, FIG. 3D, FIG. 3E, FIG. 3F, FIG. 3G, FIG. 3H, and FIG. 31 demonstrate that MHC II aAPCs promote CD4⁺ T cell cytotoxicity. (FIG. 3A) Schematic of direct CD4⁺ T cell lysis of target cells. Created with BioRender.com. (FIG. 3B) Granzyme B (GzmB) staining in OT-II cells stimulated with S1+S2 aAPCs for 7 days in media containing: no cytokines, TF, or a Th1 mix. (FIG. 3C) Day 7 GzmB levels of OT-II cells stimulated in Th1 media with S1/2, S1+S2, or αCD3/αCD28 aAPCs versus peptide pulsed OT-II splenocytes or bone-marrow derived dendritic cells (BMDCs). (FIG. 3D) Specific lysis of B16- OVA tumor cells after overnight incubation with naive or aAPC stimulated OT-II cells (cultured in TF or Th1 media). Various effector to target (E:T) ratios are presented. (FIG. 3E) Specific lysis of B16-OVA cells after overnight incubation with aAPC-stimulated and Th1-skewed OT-II cells with MHC II antibody blocking or Z-AAD-CMK GzmB inhibition. (FIG. 3F) Percentage of MHC II-expressing live B16-OVA cells after overnight incubation with aAPC-stimulated Th1 OT-II cells and MHC II or IFN-7R antibody blocking. (FIG. 3G) Experimental overview of in vivo killing and cytokine production assays on naive vs. aAPC activated Th1 OT-II cells. (FIG. 3H-FIG. 31) Specific lysis of OVA₃₂₃₋₃₃₉ pulsed splenocytes six days after adoptive T cell transfer (ACT) of naive or Th1 OT-II cells. Data in (FIG. 3B-FIG. 3F, FIG. 31) represent mean±standard error of the mean (s.e.m.) from three or more independent experiments. (FIG. 3B) n=3 mice, (FIG. 3C) n=4 (S1/2), 5 (Naive), or 6 (No Stim., Spleen APCs, BMDCs, S1+S2) mice, (FIG. 3D) n=3 mice, (FIG. 3E) n=3 (iso, MHC II, Z-AAD-CMK) or 4 (Th1 OT-II) mice, (FIG. 3F) n=3 mice, (FIG. 31) n=4 mice/group, analyzed using a (FIG. 3B-FIG. 3C) one-way or (FIG. 3D, FIG. 3F, FIG. 31) two-way ANOVA with Tukey's multiple-comparisons test;

FIG. 4A, FIG. 4B, FIG. 4C, FIG. 4D, FIG. 4E, FIG. 4F, FIG. 4G, and FIG. 4H demonstrate that MHC II aAPCs modulate CD4⁺ T cell helper function. (FIG. 4A) Schematic showing separate (I+II) or joint presentation (I/II) of MHC I and MHC II on aAPCs to CD4⁺ and CD8⁺ T cells to facilitate cell-cell crosstalk. Created with BioRender.com. (FIG. 4B-FIG. 4H) OT-I CD8⁺ T cells were activated with MHC I K_{OVA}^{b} aAPCs in TF supplemented media either alone or in co-culture with naive or aAPC-stimulated Th1 OT-II cells and MHC II I-A_{b}^{OVA} aAPCs. On day 7, (FIG. 4B) IL-7Rα (CD127) surface expression, (FIG. 4C) intracellular Granzyme B, (FIG. 4D) cytokine production, and (FIG. 4E) specific lysis of B16-OVA tumor cells after overnight incubation with CD8⁺ T cells were compared between stimulation cohorts. (FIG. 4F) Experimental overview of subcutaneous (s.c.) B16-OVA melanoma adoptive transfer model. (FIG. 4G) Tumor growth and (FIG. 4H) survival in mice subjected to adoptive transfer of OT-I CD8+ T cells that were either freshly harvested, activated in isolation, or co-activated with Th1 OT-1I CD4⁺ T cells. The black arrows indicate time of ACT. Data in (FIG. 4B, FIG. 4D-FIG. 4E) represent mean±standard error of the mean (s.e.m.) or (FIG. 4G) mean±standard deviation (s.d.) from three or more independent experiments. (FIG. 4B) n=4 (OT-I+naive OT-II) or 6 (OT-I stim, OT-I+Th1 OT-II) mice, (FIG. 4D) n=3 mice, and (FIG. 4E) n=3 (OT-I+naive OT-II, OT- I+Th1 OT-II) or 4 (OT-I stim, Naive OT-I) mice analyzed using a (FIG. 4B) one-way or (FIG. 4D-FIG. 4E) two-way ANOVA with Tukey's multiple comparisons test, (FIG. 4G-FIG. 4H) n=6 mice/group analyzed using (FIG. 4G) a repeated measure two-way ANOVA with Tukey's multiple comparisons test or (FIG. 4H) log-rank test;

FIG. 5A, FIG. 5B, FIG. 5C, FIG. 5D, FIG. 5E, FIG. 5F, FIG. 5G, FIG. 5H, FIG. 5I, and FIG. 5J demonstrate that aAPC mediated T cell help is driven by soluble factors and extends to endogenous CD8⁺ T cells. (FIG. 5A) Epifluorescent imaging and (FIG. 5B) co-localization analysis of OT-I cells (green) with naive or Th1 OT-II cells (red) and MHC 1/II aAPCs 24 hours post co-incubation. Scale bar: 100 m. (FIG. 5C) Transmigration of OT-I cells towards naive or Th1 OT-II cells relative to basal medium. (FIG. 5D) Day 7 intracellular cytokine production of OT-I cells activated alone, separated (sep.) from, or mixed (mix.) with Th1 OT-II cells in a transwell plate. (FIG. 5E) Cytokine array heatmap depicting secreted proteins from unstimulated or re-stimulated Th1 OT-II cells. (FIG. 5F) CD127 expression of OT-I cells co-cultured with Th1 OT-II cells with blocking antibodies targeting IL-10 and TNF-α. (FIG. 5G) CD127 expression of OT-I cells stimulated in IL-10 or TNF-α supplemented media. (FIG. 5H-FIG. 5J) K_{SIY}^{b}, K_{OVA}^{b}, K_{Trp2}^{b}, and D_{gp100}^{b} specific CD8⁺ T cells were enriched from B6 mice and then expanded either alone or in co-culture with Th1 OT-II cells. (FIG. 5H) Representative flow plots, (FIG. 5I) memory phenotype, and (FIG. 5J) overall cytokine production of antigen-specific CD8⁺ T cells on day 7. Data in (FIG. 5B-FIG. 5D, FIG. 5F-FIG. 5G, FIG. 5I-FIG. 5J) represent mean±standard error of the mean (s.e.m.) from three or more independent experiments. (FIG. 5B) n=8, (FIG. 5C) n =4 mice, (FIG. 5D) n=3 (OT-I+Th1 OT-II sep.) or 5 (OT-I stim, OT-I+Th1 OT-II mix.) mice, (FIG. 5E) n=4 mice, (FIG. 5F) n=4 (OT-I+Th1 OT-II, OT-I+Th1 OT-II⁺αTNFα) or 5 (OT-I+Th1 OT-II⁺αIL-10) mice, (FIG. 5G) n=5 (OT-I+TNFα) or 7 (OT-I stim, OT-I+IL-10) mice, and (FIG. 5I-FIG. 5J) n=3 mice analyzed using an (FIG. 5B) unpaired Student's t test, two-tailed, (FIG. 5C, FIG. 5F-FIG. 5G) one-way, or (FIG. 5D-FIG. 5E, FIG. 5I-FIG. 5J) two-way ANOVA with Tukey's multiple-comparisons test, mice;

FIG. 6A, FIG. 6B, FIG. 6C, FIG. 6D, FIG. 6E, FIG. 6F, and FIG. 6G demonstrate that HLA II aAPCs stimulate functional antigen-specific human CD4⁺ T cells. (FIG. 6A) HLA II aAPC design includes HLA II molecules with cleavable thrombin linkers to facilitate peptide exchange. Created with BioRender.com. (FIG. 6B) CD69 induction on HA1.7 TCR transfected Jurkat cells following stimulation with αCD3/αCD28 microparticles or a titration of DR1/αCD28 aAPCs loaded with cognate hemagglutinin (DR1 HA) or non-cognate CLIP (DR1 CLIP) peptides. (FIG. 6C-FIG. 6G) Expansion of HA-specific CD4⁺ T cells from DRB1*04:01 healthy donor peripheral blood mononuclear cells (PBMC) treated with DR4 HA aAPCs in media supplemented with four different cytokine mixes: (i) IL-2; (ii) IL-2, IL-4, IL-6, IL-1, and IFN-γ; (iii) IL-2 and IL-12; and (iv) IL-2, IL-7, and IL-15. (FIG. 6C) Representative tetramer staining, (FIG. 6D) frequency and (FIG. 6E) fold expansion of DR4 HA CD4⁺ T cells on days 0,7,14, and 21. (FIG. 6F) Memory phenotype and (FIG. 6G) intracellular cytokine production of HA-specific CD4⁺ T cells on days 14 and 21. Data in (FIG. 6B, FIG. 6D-FIG. 6G) represent mean±standard error of the mean (s.e.m.) from three or more independent experiments. (FIG. 6B) n=4 and (FIG. 6D-FIG. 6G) n=3 donors analyzed using a (FIG. 6D-FIG. 6E) repeated measure or (FIG. 6B, FIG. 6F-FIG. 6G) ordinary two-way ANOVA with Tukey's multiple-comparisons test;

FIG. 7A, FIG. 7B, FIG. 7C, FIG. 7D, FIG. 7E, FIG. 7F, FIG. 7G, FIG. 7H, FIG. 7I, FIG. 7J, FIG. 7K, FIG. 7L, FIG. 7M demonstrate characterization and function of MHC II aAPCs. (FIG. 7A) Size distribution of MHC II aAPCs as measured by dynamic light scattering (DLS). (FIG. 7B) Transmission electron imaging of MHC II aAPCs. Scale bar: 500 nm. (FIG. 7C) CFSE dilutions and (FIG. 7D) percentage of OT-II CD4⁺ T cells divided after 3 days of stimulation with a titration of I-A_{OVA}^{b} S1/2 aAPCs compared to polyclonal αCD3/αCD28 or I-A_{CLIP}^{b} aAPCs. (FIG. 7E) CFSE dilutions and (FIG. 7F) percentage of OT-II cells divided after 3 days of stimulation with I-A^{b} O_{VA} S1 aAPCs and a titration of S2, compared to S1/2 or αCD3/αCD28 aAPCs. (FIG. 7G) Representative day 7 cytokine staining of OT-II cells stimulated with S1/2 aAPCs in media containing: no cytokines, IL-2, T cell growth factor (TF) cytokine cocktail, or a Th1 mix (IL-2, IL-12p70, IFN-γ). (FIG. 7H-FIG. 7I) Fold proliferation and representative day 7 cytokine staining of OT-II cells stimulated with saturating doses of S1/2, S1+S2, or αCD3/αCD28 aAPCs versus peptide pulsed OT-II splenocytes or bone-marrow derived dendritic cells (BMDCs). (FIG. 7J) Fluorescent quantification of I-A^{b} O_{VA} on 300 nm nanoparticles conjugated with S1, S1 and αCD28 (S1/2) at a 1:1 ratio, S1 and isotype antibodies (S1/I), or S1 and BSA (S1/B) at 1:1 and 1:3 ratios. (FIG. 7K) Day 3 CFSE, (FIG. 7L) day 7 fold proliferation, and (FIG. 7M) day 7 cytokine secretion of OT-II CD4⁺ T cells stimulated with S1/2, S1, S1/I, and S1/B nanoparticles with soluble S2, or S1/2 4.5 m microparticles. Data in (a-b) are representative of two independent samples. Data in (FIG. 7D, FIG. 7F, FIG. 7H, FIG. 7J- FIG. 7M) represent mean±standard error of the mean (s.e.m.) from three or more independent experiments. (FIG. 7D) n=4 mice, (FIG. 7F) n=3 mice, (FIG. 7H) n=4 (CLIP) or 7 (No Stim., Spleen APCs, BMDCs, αCD3/αCD28, S1/2, S1+S2) mice, (FIG. 7I) n=4, (FIG. 7J-FIG. 7K) n=6 mice, (FIG. 7L) n=3 mice, analyzed using a (FIG. 7D, FIG. 7F) one-way ANOVA compared to no stim. condition with Dunnet's multiple-comparisons test, an (FIG. 7H, FIG. 7J-FIG. 7L) ordinary one-way, or a (FIG. 7M) two-way ANOVA with Tukey's multiple-comparisons test;

FIG. 8A, FIG. 8B, FIG. 8C, FIG. 8D, FIG. 8E, FIG. 8F, FIG. 8G, FIG. 8H, and FIG. 81 demonstrate that antigen-specific MHC II aAPC internalization enhances CD4⁺ T cell magnetic enrichment. (FIG. 8A) Fold enrichment and (FIG. 8B) percent cell recovery of OT-II cells diluted into a B6 background at a ratio of 1:1000 after magnetic enrichment with S1 aAPCs after 2 hours of incubation at various temperatures with and without sodium azide (NaN₃) uptake inhibitor. (FIG. 8C- FIG. 8E) Binding and internalization of PE-labelled S 1 aAPCs by OT-II (cog.) and B6 (non-cog) CD4⁺ T cells after incubation for 30 minutes and 2 hours at various temperatures with and without NaN₃. CD4⁺ T cells with particles on their surface are Tet⁺MHC II⁺, whereas cells with internalized particles are Tet⁺MHC II. (FIG. 8C) Representative flow plots of CD4⁺ T cells that have either bound or internalized particles, (FIG. 8D) Representative flow plots and (FIG. 8E) overall MHC II and TCRβ staining of the Tet* CD4⁺ T cells from (FIG. 8C). (FIG. 8F) Pearson's correlation of MHC II detection and particle fluorescence from (FIG. 8G) confocal imaging of OT-1I CD4⁺ T cells incubated with AF488- labelled S1 aAPCs after incubation for 2 hours at various temperatures with and without NaN₃. Scale bar: 4 m. (FIG. 8H-FIG. 81) Particle internalization tracking after magnetic enrichment of OT-II cells diluted into a B6 background at a ratio of 1:1000 with PE-labelled S 1 aAPCs after incubation for 30 minutes and 2 hours at various temperatures with and without NaN₃. (FIG. 8H) Representative flow plots and (FIG. 81) overall MHC II and PE staining of enriched OT-II, enriched B6, or unenriched OT-1I CD4⁺ T cell populations from the enrichment experiments. Data in (FIG. 8A-FIG. 8B, FIG. 8E-FIG. 8F, FIG. 81) represent mean±standard error of the mean (s.e.m.) from three or more independent experiments. (FIG. 8A) n=3 (37° C.+NaN₃) or 5 (4° C., 37° C.−NaN₃) mice, (FIG. 8E) n=3 mice, (FIG. 8F) n=3 (37° C.+NaN₃) or 4 (4° C., 37° C.—NaN₃), (FIG. 81) n=3 mice, analyzed using a one-way (FIG. 8A-FIG. 8B, FIG. 8F) or two-way ANOVA (FIG. 8E, FIG. 81) with Tukey's multiple-comparisons test;

FIG. 9A, FIG. 9B, FIG. 9C, FIG. 9D, FIG. 9E, FIG. 9F, FIG. 9G, and FIG. 9H demonstrate the impact of MHC II aAPC size, ligand density, and dosing on antigen-specific CD4⁺ T cell binding and enrichment. (FIG. 9A-FIG. 9B) Particle binding to OT-II (cog.) and B6 (non-cog.) CD4⁺ T cells after incubation at 30 minutes and 37° C. with 300 nm nanoparticles conjugated with S1, S1 and αCD28 (S1/2) at a 1:1 ratio, S 1 and isotype antibodies (S1/I) or BSA (S 1/B) at 1:1 or 1:3 ratios, or with S1/2 4.5 m microparticles. (FIG. 9A) Representative flow plots at 30 ng I-Ab/10⁵ CD4⁺ T cells, and (FIG. 9B) Percent cells bound across a range of doses. (FIG. 9C) OT-II CD4⁺ T cells were diluted 1:1000 into a B6 background and incubated for 2 hours at 37° C. with 30 ng I-Ab/10⁶ CD4⁺ T cells of S1/2, S1, or S1/I 1:1 nano-aAPCs versus S1/2 micro-aAPCs. Fold enrichment of magnetically enriched samples relative to baseline. (FIG. 9D) Representative flow plots of OT-II (top) and SMART-A1 CD4⁺ T cells (bottom) pre and post-enrichment, (FIG. 9E-FIG. 9F) fold enrichment and percent cell recovery of (FIG. 9E) OT-II and (FIG. 9F) SMART-A1 cells post-enrichment with a titration of cognate S1 nano-aAPCs. (FIG. 9G) Total number of CD4⁺ T cells and (FIG. 9H) percentage of I-A_{OVA}^{b} tetramer positive CD4⁺ T cells 7 days after S1/2 or S1+S2 enrichment and expansion. Data in (FIG. 9B, FIG. 9C, FIG. 9E-FIG. 9H) represent mean±standard error of the mean (s.e.m.) from three or more independent experiments. (FIG. 9B- FIG. 9C) n=3 mice, (FIG. 9E) n=3 (3-30 ng) or 5 (60-480 ng) mice, (FIG. 9F) n=3 mice, and (FIG. 9G- FIG. 9H) n=3 (S1/2) or 4 (S1+52) mice analyzed using a (FIG. 9B) two-way or (FIG. 9C, FIG. 9E-FIG. 9F) one-way ANOVA with Tukey's multiple-comparisons test, or (FIG. 9G-FIG. 9H) an unpaired Student's t test.

FIG. 10A, FIG. 10B, FIG. 10C, FIG. 10D, FIG. 10E, FIG. 10F. FIG. 10G, FIG. 10H, FIG. 101, FIG. 10J, FIG. 10K, and FIG. 10L demonstrate that MHC II aAPCs promote CD4⁺ T cell cytotoxicity. (FIG. 10A) Day 7 GzmB levels in OT-II cells stimulated with S1+S2 aAPCs in media containing: no cytokines, TF, or a Th1 mix (IL-2, IL-12p70, IFN-γ). (FIG. 10B) Day 7 GzmB levels of OT-II cells stimulated in Th1 media with S1/2, S1+S2, or αCD3/αCD28 aAPCs versus peptide pulsed OT-II splenocytes or bone-marrow derived dendritic cells (BMDCs). (FIG. 10C) OT-II cytokine production on days 0, 3, 5, 7 of stimulation with S1 aAPCs in Th1 media. (FIG. 10D) GzmB staining and (FIG. 10E) percent positive of OT-II cells after 7 days of S1+S2 stimulation in the various components of the Th1 mix. (FIG. 10F) Percentage of GzmB+OT-II cells after 7 days of stimulation with 300 nm nanoparticles conjugated with S1, S1 and αCD28 (S1/2) at a 1:1 ratio, S1 and isotype antibodies (S1/I) or BSA (S 1/B) at 1:1 or 1:3 ratios, or S1/2 4.5 m microparticles. (FIG. 10G) B16-OVA tumor cell viability after overnight incubation at an effector-to-target (E:T) ratio of 30:1 with naive or aAPC stimulated OT-II cells cultured in TF or Th1 media. (FIG. 10H) Live B16-OVA MHC II expression after overnight incubation with aAPC stimulated Th1 OT-II cells and MHC II or IFN-γR antibody blocking. (FIG. 101) T-bet staining and (FIG. 10J) percentage, (FIG. 10K) IFN-γ and TNF-α staining and (FIG. 101) percentage of naive versus Th1 OT-II CD4⁺ T cells 21 days post adoptive cell transfer (ACT). Data in (FIG. 10C, FIG. 10E, FIG. 10F, FIG. 10J, FIG. 10L) represent mean±standard error of the mean (s.e.m.). (FIG. 10c) n=3 (D3) or 4 (DO, D5, D7) mice, (FIG. 10E) n=3 mice, (FIG. 10F) n=6 mice, analyzed using a (FIG. 10C) two-way or (FIG. 10E-FIG. 10F) one-way ANOVA with Tukey's multiple-comparisons test, (FIG. 10J, FIG. 10L) n=2 mice/group analyzed using an (FIG. 10J) unpaired Student's t test, two-tailed or (FIG. 101) two-way ANOVA with Tukey's multiple- comparisons test;

FIG. 11A, FIG. 11B, FIG. 11C, FIG. 11D, FIG. 11E, FIG. 11F, FIG. 11G, FIG. 11H, FIG. 111, FIG. 1 J, FIG. 11K, FIG. 11L, and FIG. 11M demonstrate that MHC II aAPCs modulate CD4⁺ T cell helper function. (FIG. 11A- FIG. 11D) OT-I cells in TF supplemented media were activated with MHC I K^{b} O_{VA} aAPCs either alone or in co-culture with naive or aAPC activated Th1 OT-II cells and MHC II I-Ae O_{VA} aAPCs. Day 7 (FIG. 11A-FIG. 11B) memory phenotype, (FIG. 11C) CD127 expression, and (FIG. 11D) cytokine staining from the various stimulations. (FIG. 11E-FIG. 11F) OT-I cells were cultured as above but with different stimuli: K^{b} O_{VA} only (MHC I), separate (MHC I+II), and co-presenting (MHC I/II) aAPCs. Day 7 (FIG. 11E-FIG. 11F) intracellular GzmB levels and (FIG. 11G) memory phenotype of OT-I cells stimulated under these various conditions. (FIG. 11H) B16-OVA viability after overnight incubation at an E:T ratio of 30:1 with OT-I cells stimulated alone or co-cultured with naive or Th1 OT-II cells. (FIG. 11I) B16-SIY and (FIG. 11J) B16-F10 specific lysis after overnight incubation with 2C or PMEL CD8⁺ T cells, respectively, stimulated alone or co-cultured with naive or Th1 OT-II cells. (FIG. 11K-FIG. 11L) Percentage of CD3+lymphocytes that are CD4⁺ or CD8⁺ T cells over five days of OT-I and Th1 OT-II co-culture. (FIG. 11M) Spider plots depicting tumor growth of B16-OVA in B6 mice subjected to adoptive transfer of OT-I cells that were either freshly isolated, activated alone, or co-activated with Th1 OT-II CD4⁺ T cells. Data in (FIG. 111B, FIG. 11F, FIG. 11G, FIG. 11I-FIG. 11K) represent mean±standard error of the mean (s.e.m.) from two or more independent experiments. (FIG. 11B) n=4 mice, (FIG. 11F) n=5 mice, (FIG. 11G) n=3 (MHC I/II) or 6 (OT-I stim, OT-I+naive OT-II, MHC I, MHC 1+II) mice, (FIG. 111- FIG. 11J) n=2 mice, and (FIG. 11k) n=3 mice, analyzed using a (FIG. 11F) one-way or (FIG. 11B, FIG. 11G) two-way ANOVA with Tukey's multiple-comparisons test;

FIG. 12A, FIG. 12B, FIG. 12C, FIG. 12D, FIG. 12E, FIG. 12F, FIG. 12G, FIG. 12H, and FIG. 121 demonstrate that aAPC mediated T cell help is driven by soluble factors and extends to endogenous CD8⁺ T cells. (FIG. 12A) Memory phenotype, (FIG. 12B) intracellular GzmB levels, and (FIG. 12C) cytokine staining of OT-I cells activated alone, separated (sep.) from, or mixed (mix.) with Th1 OT-II cells in a transwell plate. (FIG. 12D) Representative cytokine arrays of supernatants harvested from unstimulated or re-stimulated Th1 OT-II cells. (FIG. 12E) Flow cytometry detection of GzmB expression in OT-I cells co-cultured with Th1 OT-II cells in the presence of blocking antibodies to IL-10 and TNF-α. (FIG. 12F) Flow cytometry detection of GzmB in OT-I cells stimulated in media supplemented with IL-10 or TNF-α. (FIG. 12G-FIG. 121) K_{SIY}^{b}, K_{OVA}^{b}, K_{Trp2}^{b}, and D_{gp100}^{b} specific CD8⁺ T cells were enriched from B6 mice and then expanded either alone or in co-culture with Th1 OT-II cells. (FIG. 12G) Dimer staining and (FIG. 12H) numbers of CD8⁺ T cells of corresponding antigenic specificities at day 7. (FIG. 121) Percent of antigen-specific CD8⁺ T cells that were CD127 positive. Data in (FIG. 12A, FIG. 12E-FIG. 12F, FIG. 12H-FIG. 121) represent mean±standard error of the mean (s.e.m.) and three or more independent experiments. (FIG. 12A) n=3 mice, (FIG. 12E) n=8 mice, (FIG. 12F) n=3 mice, and (FIG. 12H-FIG. 121) n=3 mice analyzed using a (FIG. 12E-FIG. 12F, FIG. 12H-FIG. 121) one-way or (FIG. 12A) two-way ANOVA with Tukey's multiple-comparisons test;

FIG. 13A, FIG. 13B, FIG. 13C, FIG. 13D, FIG. 13E, and FIG. 13F demonstrate that HLA II aAPCs stimulate functional antigen-specific human CD4⁺ T cells. (FIG. 13A-FIG. 13B) SDS-PAGE analysis of human embryonic kidney (HEK) 293-F cell15 secreted (FIG. 13A) DR1 and (FIG. 13B) DR4 monomers. (FIG. 13C) Detection of HA 1.7 TCR on Jurkat cells after overnight transfection and (FIG. 13D) comparison of CD69 induction on HA1.7 TCR positive and negative Jurkat cells following stimulation with either αCD3/αCD28 microparticles or a titration of DR1/αCD28 aAPCs loaded with cognate hemagglutinin (DR1 HA) or non-cognate CLIP (DR1 CLIP) peptides. (FIG. 13E) Memory phenotype and (FIG. 13F) intracellular cytokine production after cognate (HA) and irrelevant (NY-ESO-1) peptide stimulation of DR4 HA tetramer positive CD4⁺ T cells expanded from healthy donor peripheral blood mononuclear cells (PBMC) using DR4 HA aAPCs and four cytokine mixes: (i) IL-2 only; (ii) IL-2, IL-4, IL-6, IL-10, and IFN-γ; (iii) IL-2 and 12; and (iv) IL-2, IL-7, and IL-15;

FIG. 14A, FIG. 14B, and FIG. 14C show representative flow cytometry gating strategies. (FIG. 14A) Gating strategy for T cell functional, phenotypic, or specificity analysis, based on sequential gating for viability markers, lymphocytes, singlets, and then T cell subsets. (FIG. 14B) Gating strategy for in vitro killing assays, based on sequential gating for CFSE labelled tumor cells and viability markers. (FIG. 14C) Gating strategy for in vivo killing assays, based on sequential gating on lymphocytes, singlets, CD45.2 and CFSE positive cells, and then either MHC II high or low subsets;

FIG. 15A, FIG. 15B, FIG. 15C, and FIG. 15D show MHC II aAPCs for CD4⁺ T cell stimulation. (FIG. 15A) Schematic of MHC II aAPCs. (FIG. 15B) Day 7 fold proliferation of OT-II CD4⁺ T cells stimulated through traditional means or MHC II aAPCs. (FIG. 15C-FIG. 15D) Day 7 cytokine production of OT-TT cells stimulated as above. (FIG. 15B) One-way or (FIG. 15D) two-way ANOVA with Tukey's post-hoc test; **p<0.01, ****p<0.0001;

FIG. 16A, FIG. 16B, FIG. 16C, and FIG. 16D show MHC II aAPCs induce CD4⁺ T cell cytotoxicity. (FIG. 16A-FIG. 16B) Day 7 GzmB levels of OT-II CD4⁺ T cells stimulated through traditional means or MHC II aAPCs. (FIG. 16C) Cytotoxicity of aAPC-stimulated OT-II cells against B16-OVA tumor cells with MHC II blockade (αMHC II) or GzmB inhibition (Z-AAD-CMK). (FIG. 16D) Day 7 GzmB levels of aAPC-stimulated OT-II cells in various components of Th1 cytokines. (FIG. 16B, FIG. 16D) One-way or (FIG. 16C) two-way ANOVA with Tukey's post-hoc test; **p<0.01, ***p<0.001, ****p<0.0001;

FIG. 17A, FIG. 17B, FIG. 17C, and FIG. 17D show mechanical cues from MHC II aAPCs influence CD4⁺ T cell cytotoxicity. (FIG. 17A-FIG. 17B) OT-II internalization of MHC II aAPCs after incubation at different temperatures with or without NaN₃ metabolic inhibition. (FIG. 17C) Degree of particle internalization versus Day 7 CD4⁺ T cell GzmB levels. (FIG. 17D) Hydrogel cross-linker density versus Day 7 CD4⁺ T cell GzmB levels. (FIG. 17B) One-way ANOVA with Tukey's post-hoc test, (FIG. 17C-FIG. 17D) F-test for linear regression; **p<0.01, ****p<0.0001; and

FIG. 18a, FIG. 18b, FIG. 18c, FIG. 18d, FIG. 18e, and FIG. 18f show production and testing of DR-Fc aAPCs. (FIG. 13a) Design of cysteine substituted (bottom) and cysteine non-substituted (top) DR-Fc fusion constructs. (FIG. 18b) Gel electrophoresis of constructs produced under non-reducing conditions in small-scale co-transfection tests of DRα and DRβ chain plasmids titrated in 1:2, 1:1, or 2:1 ratios. Lane 1: DR1-Fc^{S473C} 1:2 (α:β); lane 2: DR1-FC^{S473C} 1:1 (α:β); lane 3: DR1-Fc^{S473C} 2:1 (α:β); lane 4: DR4-Fc^{S473C} 1:2 (α:β); lane 5: DR4-FC^{S473C}1:1 (α:β); lane 6: DR4-Fc^{S473C}2:1 (α:β); lane 7: DR1-Fc; lane 8: DR4-Fc. (FIG. 18c) Gel electrophoresis of constructs produced under reducing conditions in small-scale co-transfection tests of DRα and DRβ chain plasmids titrated in 1:2. 1:1, or 2:1 ratios. Lane 1: DR1-Fc^{S473C} 1:2 (α:); lane 2: DR1-Fc^{S473C} 1:1 (α: P); lane 3: DR1-Fc^{S473C}2:1 (α:β); lane 4: DR4-Fc^{S473C} 1:2 (α: p); lane 5: DR4-Fc^{S473C}1:1 (α:); lane 6: DR4-Fc^{S473C} 2:1 (α:β); lane 7: DR1-Fc; lane 8: DR4-Fc. (FIG. 18d) Large-scale preparation (purified and concentrated) of DR1-Fc and DR4-Fc constructs produced under reducing and non-reducing conditions. Lane 1: DR1-Fc non-reducing; lane 2: DR1-Fc reducing; lane 3: DR4-Fc non-reducing; lane 4: DR4-Fc reducing; lane 5: DR1-Fc^{S473C} non-reducing: lane 6: DR1-Fc^{S473C}reducing; lane 7: DR4-Fc^{S473C} non-reducing; lane 8: DR4-Fe^{S473C} reducing. (FIG. 18e) Flow cytometry results of HA1.7+(left) and HA1.7- (right) Jurkat cells induced with DR4 aAPC, DR1-Fc^{S473C} aAPC, and DR4-Fc^{S473C} aAPC. Anti-CD3 antibody and non-stimulated Jurkat cells are shown as controls. (FIG. 18f) Quantification of results shown in FIG. 18e.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. MAJOR HISTOCOMPATIBILITY COMPLEX CLASS II (MHC II) ARTIFICIAL ANTIGEN PRESENTING CELLS HARNESS THE EFFECTOR AND HELPER FUNCTIONS OF ANTIGEN-SPECIFIC CD4⁺ T CELLS

In some embodiments, the presently disclosed subject matter provides an artificial antigen presenting cell (aAPC) comprising a particle having a major histocompatibility complex class II (MHC II) molecule conjugated to a surface thereof. In other embodiments, the presently disclosed subject matter provides an artificial antigen presenting cell (aAPC) consisting essentially of a particle having a major histocompatibility complex class II (MHC II) molecule conjugated to a surface thereof.

In certain embodiments, the MHC II molecule comprises an MHC II I-A^{b} monomer.

In certain embodiments, the aAPC further comprises a costimulatory ligand conjugated to a surface thereof. In particular embodiments, the costimulatory ligand is selected from the group consisting of an antibody or antigen-binding fragment thereof that specifically binds to CD28, CD80 (B7-1), CD86 (B7-2), B7- H3, 4-1BBL, 4-1BB, CD27, CD30, CD134 (OX-40L), B7h (B7RP-1), CD40, LIGHT, an antibody or antigen-binding fragment thereof that specifically binds to HVEM, an antibody or antigen-binding fragment thereof that specifically binds to CD40L, an antibody or antigen binding fragment thereof that specifically binds to OX40, and an antibody or antigen-binding fragment thereof that specifically binds to 4-1BB. In more particular embodiments, the costimulatory ligand comprises an anti-CD28 (αCD28) antibody.

In some embodiments, the aAPC further comprises a major histocompatibility complex class I molecule conjugated to a surface thereof. In certain embodiments, the MHC-class I molecule comprises a K^{b}-Ig dimer.

In certain embodiments, the MHC II molecule comprises a human leukocyte antigen (HLA) class II monomer. In certain embodiments, the HLA class II monomer is selected from the group consisting of HLA-DR, HLA-DP, and HLA-DQ. In certain embodiments, the HLA class II monomer comprises DR1 or DR4. In particular embodiments, the HLA class II monomer comprises a cleavable thrombin linker, wherein the cleavable thrombin linker enables peptide exchange.

In certain embodiments, the HLA class II monomer comprises DR1 fused to an Fc domain. In certain embodiments, the Fc domain comprises an amino acid sequence comprising a cysteine substitution. In certain embodiments, the Fc domain comprises a cysteine at position 473.

In certain embodiments, the HLA class II monomer comprises DR4 fused to an Fc domain. In certain embodiments, the Fc domain comprises an amino acid sequence comprising a cysteine substitution. In certain embodiments, the Fc domain comprises a cysteine at position 473.

In some embodiments, the presently disclosed subject matter provides an aAPC having an HLA class I molecule and an HLA class II molecule conjugated to a surface thereof. In certain embodiments, the aAPC comprises DR1 or DR4 monomers with HLA A2-Ig dimers. In certain embodiments, the HLA class II monomer comprises DR1 fused to an Fc domain. In certain embodiments, the Fc domain comprises an amino acid sequence comprising a cysteine substitution. In certain embodiments, the Fc domain comprises a cysteine at position 473. In certain embodiments, the HLA class II monomer comprises DR4 fused to an Fc domain. In certain embodiments, the Fc domain comprises an amino acid sequence comprising a cysteine substitution. In certain embodiments, the Fc domain comprises a cysteine at position 473.

In some embodiments, the particle comprises a paramagnetic particle. In particular embodiments, the particle comprises an iron-dextran particle.

In other embodiments, the presently disclosed subject matter provides a method for identifying, isolating, or detecting one or more antigen-specific T cells, the method comprising:
(a) contacting a plurality of unpurified immune cells comprising one or more antigen-specific T cells with a plurality of aAPCs of any one of claims 1-27;
(b) separating antigen-specific T cells associated with the plurality of aAPCs from cells not associated with the plurality of aAPCs;
(c) recovering antigen-specific T cells associated with the plurality of aAPCs; and
(d) expanding the recovered antigen-specific T cells in culture for a period of time to provide a composition comprising antigen-specific T cells.

In some embodiments, the plurality of unpurified immune cells comprising one or more antigen-specific T cells are obtained from a sample comprising one or more of a peripheral blood mononuclear cell (PBMC) sample, memory T cells, naive T cells, previously activated T cells, and tumor infiltrating lymphocytes. In some embodiments, the plurality of unpurified immune cells comprising one or more antigen-specific T cells are obtained from a sample comprising one or more of bone marrow, lymph node tissue, spleen tissue, and a tumor.

In certain embodiments, the plurality of unpurified immune cells are obtained from a patient or a donor. In certain embodiments, the donor comprises a donor who is HLA-matched to an adoptive transfer recipient. In certain embodiments, the plurality of unpurified immune cells are obtained from a patient and the patient has one or more diseases, disorders, or conditions selected from the group consisting of a cancer, an infectious disease, and an autoimmune disease.

In some embodiments, the one or more antigen-specific T cells are selected from the group consisting of cytotoxic CD4⁺ T cells, CD4⁺ helper T cells, CD8⁺ cytotoxic T lymphocytes, T-helper 17 (Th17) cells, regulatory T cells (Tregs), and combinations thereof.

In certain embodiments, the expanding of the recovered cells in culture for a period of time is performed on a multi-well microtiter plate. In particular embodiments, the multi-well microtiter plate comprises a 96-well microtiter plate.

In some embodiments, a purity of the expanded recovered antigen-specific T cells is improved relative to a method in which the antigen-specific T cells are isolated from the plurality of unpurified immune cells prior to contacting the plurality of unpurified immune cells with the plurality of aAPCs.

In some embodiments, a percent of antigen-specific T cells is increased relative to a method in which the antigen-specific T cells are isolated from the plurality of unpurified immune cells prior to contacting the plurality of unpurified immune cells with the plurality of aAPCs.

In some embodiments, a number of antigen-specific T cells is increased relative to a method in which the antigen-specific T cells are isolated from the plurality of unpurified immune cells prior to contacting the plurality of unpurified immune cells with the plurality of aAPCs.

In certain embodiments, the plurality of aAPCs comprise or consist essentially of a particle having a major histocompatibility complex class II (MHC II) molecule conjugated to a surface thereof.

In certain embodiments, the method further comprises ex vivo generation of cytotoxic CD4⁺ T cells.

In some embodiments, the method further comprises administering a soluble costimulatory ligand to the antigen-specific T cells associated with the plurality of aAPCs after step (b).

In certain embodiments, the costimulatory ligand is selected from the group consisting of an antibody or antigen-binding fragment thereof that specifically binds to CD28, CD80 (B7-1), CD86 (B7-2), B7- H3, 4-1BBL, 4-1BB, CD27, CD30, CD134 (OX-40L), B7h (B7RP-1), CD40, LIGHT, an antibody or antigen-binding fragment thereof that specifically binds to HVEM, an antibody or antigen-binding fragment thereof that specifically binds to CD40L, an antibody or antigen binding fragment thereof that specifically binds to OX40, and an antibody or antigen-binding fragment thereof that specifically binds to 4-1BB. In particular embodiments, the costimulatory ligand comprises an anti-CD28 (αCD28) antibody.

In certain embodiments, the method further comprises administering one or more cytokines to the plurality of unpurified immune cells comprising one or more antigen-specific T cells. In particular embodiments, the one or more cytokines include one or more of IL-2, IL-12p70, and IFN-7.

In certain embodiments, the method further comprises incubating the plurality of unpurified immune cells comprising one or more antigen-specific T cells contacted with a plurality of aAPCs for a period of time at a predetermined temperature. In particular embodiments, the period of time is about 2 hr. In particular embodiments, the predetermined temperature is about 37° C.

In some embodiments, the method comprises a ratio of major histocompatibility complex class II (MHC II) molecule to CD4⁺ T cells is about 30 ng MHC II/106 CD4⁺ T cells.

In some embodiments, the aAPC comprises a particle having a major histocompatibility complex class II (MHC II) molecule and major histocompatibility complex class I molecule conjugated to a surface thereof. In certain embodiments, the method co-activates CD4⁺ and CD8⁺ T cells. In certain embodiments, the co-activation of CD4⁺ and CD8⁺ T cells enhances the therapeutic function and memory formation of the CD8⁺ T cells.

In some embodiments, the method comprises redirecting CD4⁺ T cell help of one specificity toward CD8⁺ T cells of a multitude of specificities.

In some embodiments, the aAPC has an HLA class I molecule and an HLA class II molecule conjugated to a surface thereof. In certain embodiments, the aAPC comprises DR1 or DR4 monomers with HLA A2-Ig dimers. In certain embodiments, the HLA class II monomer comprises DR1 fused to an Fc domain. In certain embodiments, the Fc domain comprises an amino acid sequence comprising a cysteine substitution. In certain embodiments, the Fc domain comprises a cysteine at position 473. In certain embodiments, the HLA class II monomer comprises DR4 fused to an Fc domain. In certain embodiments, the Fc domain comprises an amino acid sequence comprising a cysteine substitution. In certain embodiments, the Fc domain comprises a cysteine at position 473.

In some embodiments, the method comprises redirecting a particular HLA class II specificity to relay help from CD4⁺ T cells of that specificity to CD8⁺ T cells of a range of specificities.

In certain embodiments, the particle comprises a paramagnetic particle. In particular embodiments, the paramagnetic particle comprises an iron-dextran particle. In certain embodiments, the separating of the antigen-specific T cells associated with the plurality of aAPCs from the cells not associated with the plurality of aAPCs is by magnetic separation.

In other embodiments, the presently disclosed subject matter provides a method for treating a disease, disorder, or condition, the method comprising administering to a subject in need of treatment thereof a composition comprising one or more antigen-specific T cells prepared by the presently disclosed methods. In certain embodiments, the disease, disorder, or condition is selected from the group consisting of a cancer, an infectious disease, and an autoimmune disease. In particular embodiments, the disease, disorder, or condition is a cancer and the one or more antigen-specific T cells comprise cytotoxic T cells specific for one or more tumor-associated peptide antigens to the subject in need of treatment thereof. In yet more particular embodiments, the cancer comprises a solid tumor or a hematological malignancy. In even yet more particular embodiments, the cancer is selected from the group consisting of a melanoma, colon cancer, duodenal cancer, prostate cancer, breast cancer, ovarian cancer, ductal cancer, hepatic cancer, pancreatic cancer, renal cancer, endometrial cancer, testicular cancer, stomach cancer, dysplastic oral mucosa, polyposis, head and neck cancer, invasive oral cancer, non-small cell lung carcinoma, small-cell lung cancer, mesothelioma, transitional and squamous cell urinary carcinoma, brain cancer, a neuroblastoma, and a glioma.

### A. Representative Sources of Immune Cells

As provided hereinabove, in some embodiments, the presently disclosed methods involve enrichment and expansion of antigen-specific T cells, including, but not limited to, cytotoxic T lymphocytes (CTLs), helper T cells, and regulatory T cells. In some embodiments, the presently disclosed methods involve enrichment and expansion of antigen-specific CTLs.

Precursor T cells can be obtained from a patient or from a suitable HLA-matched donor. Precursor T cells can be obtained from a number of sources, including, but not limited to, peripheral blood mononuclear cells (PBMC), bone marrow, lymph node tissue, spleen tissue, tumors, and combinations thereof. In some embodiments, the T cells are obtained from a PBMC sample from a patient. In some embodiments, the PBMC sample is used to isolate the T cell population of interest, such as CD8⁺, CD4⁺ or regulatory T cells. In some embodiments, precursor T cells are obtained from a unit of blood collected from a patient or a donor using any number of techniques known to the skilled artisan, such as Ficoll separation. For example, precursor T cells from the circulating blood of a patient or a donor can be obtained by apheresis or leukapheresis. The apheresis product typically contains lymphocytes, including T cells and precursor T cells, monocytes, granulocytes, B cells, other nucleated white blood cells, red blood cells, and platelets. Leukapheresis is a laboratory procedure in which white blood cells are separated from a sample of blood.

Cells collected by apheresis can be washed to remove the plasma fraction and to place the cells in an appropriate buffer or media for subsequent processing steps. Washing steps can be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample can be removed, and the cells directly re-suspended in a culture medium.

If desired, precursor T cells can be isolated from peripheral blood lymphocytes by lysing the red blood cells and depleting the monocytes, for example, by centrifugation through a PERCOLL™ gradient. In certain embodiments, the sample from which the T cells are obtained can be used without any isolation or preparatory steps.

If desired, subpopulations of T cells can be separated from other cells that may be present. For example, specific subpopulations of T cells, such as CD28+, CD4+, CD8+, CD45RA+, and CD45RO+ T cells, can be further isolated by positive or negative selection techniques. Other enrichment techniques include cell sorting and/or selection via negative magnetic immunoadherence or flow cytometry, e.g., using a cocktail of monoclonal antibodies directed to cell surface markers present on the cells negatively selected.

In certain embodiments, leukocytes are collected by leukapheresis, and are subsequently enriched for CD8⁺ T cells using known processes, such as magnetic enrichment columns that are commercially available. The CD8-enriched cells are then enriched for antigen-specific T cells using magnetic enrichment with the aAPC reagent. In various embodiments, at least about 10⁵, or at least about 10⁶, or at least about 10⁷ CD8-enriched cells are isolated for antigen-specific T cell enrichment.

B. Artificial Antigen Presenting Cells (aAPCs) Comprising Magnetic Particles

Representative methods for preparing aAPCs are provided in International PCT Patent Application No. PCT/US21/38676 for Adaptive Nanoparticle Platforms for High Throughput Expansion and Detection of Antigen-Specific T Cells, filed Jun. 23, 2021, which is incorporated herein in its entirety.

As provided hereinabove, the sample comprising the immune cells (e.g., CD4⁺ T cells and/or CD8⁺ T cells) is contacted with an artificial Antigen Presenting Cell (aAPC) comprising a particle having magnetic properties. In some embodiments, such particles are nanoparticles and are referred to herein as “nano-aAPCs.” Paramagnetic materials have a small, positive susceptibility to magnetic fields. These materials are attracted by a magnetic field and the material does not retain the magnetic properties when the external field is removed. Exemplary paramagnetic materials include, without limitation, magnesium, molybdenum, lithium, tantalum, and iron oxide. Paramagnetic beads suitable for magnetic enrichment are commercially available (e.g., DYNABEADS®, MACS MICROBEADS™ Miltenyi Biotec, and the like). In some embodiments, the aAPC particle comprises an iron dextran bead (e.g., a dextran-coated iron-oxide bead).

In certain embodiments, the aAPCs contain at least two ligands, an antigen presenting complex (e.g.. a major histocompatibility complex (MHC), including a peptide-MHC), and a costimulatory ligand, e.g., a lymphocyte activating ligand. Antigen presenting complexes comprise an antigen binding cleft, which harbors an antigen for presentation to a T cell or T cell precursor. Antigen presenting complexes can be, for example, MHC class I or class II molecules, and can be linked or tethered to provide dimeric or multimeric MHC. In some embodiments, the MHC are monomeric, but their close association on the paramagnetic nanoparticle is sufficient for avidity and activation. In some embodiments, the MHC are dimeric. Dimeric MHC class I constructs can be constructed by fusion to immunoglobulin heavy chain sequences, which are then associated through one or more disulfide bonds (and with associated light chains). In some embodiments, the signal 1 complex is a non-classical MHC-like molecule, such as member of the CD1 family (e.g., CD1a, CD1b, CD1c, CD1d, and CD1e). MHC multimers can be created by direct tethering through peptide or chemical linkers, or can be multimeric via association with streptavidin through biotin moieties. In some embodiments, the antigen presenting complexes are MHC class I or MHC class II molecular complexes involving fusions with immunoglobulin sequences, which are extremely stable and easy to produce, based on the stability and secretion efficiency provided by the immunoglobulin backbone.

MHC class I molecular complexes having immunoglobulin sequences are described in U.S. Pat. No. 6,268,411, which is hereby incorporated by reference in its entirety. These MIIC class I molecular complexes may be formed in a conformationally intact fashion at the ends of immunoglobulin heavy chains. MHC class I molecular complexes to which antigenic peptides are bound can stably bind to antigen-specific lymphocyte receptors (e.g., T cell receptors). In various embodiments, the immunoglobulin heavy chain sequence is not full length, but comprises an Ig hinge region, and one or more of CH1, CH2, and/or CH3 domains. The Ig sequence may or may not comprise a variable region, but where variable region sequences are present, the variable region may be full or partial. The complex may further comprise immunoglobulin light chains.

Exemplary MHC class I molecular complexes comprise at least two fusion proteins. A first fusion protein comprises a first MHC class I a chain and a first immunoglobulin heavy chain (or portion thereof comprising the hinge region), and a second fusion protein comprises a second MHC class I a chain and a second immunoglobulin heavy chain (or portion thereof comprising the hinge region). The first and second immunoglobulin heavy chains associate to form the MHC class I molecular complex, which comprises two MHC class I peptide-binding clefts. The immunoglobulin heavy chain can be the heavy chain of an IgM, IgD, IgG1, IgG3, IgG2P, IgG2a, IgG4, IgE, or IgA. In some embodiments, an IgG heavy chain is used to form MHC class I molecular complexes. If multivalent MHC class I molecular complexes are desired, IgM or IgA heavy chains can be used to provide pentavalent or tetravalent molecules, respectively. Exemplary class I molecules include HLA-A, HLA-B, HLA-C, HLA-E, and these may be employed individually or in any combination. In some embodiments, the antigen presenting complex is an HLA-A2 ligand.

Exemplary MHC class II molecular complexes are described in U.S. Pat. Nos. 6,458,354, 6,015,884, 6,140,113, and 6,448,071, which are hereby incorporated by reference in their entireties. MHC class II molecular complexes comprise at least four fusion proteins. Two first fusion proteins comprise (i) an immunoglobulin heavy chain (or portion thereof comprising the hinge region) and (ii) an extracellular domain of an MHC class 11 chain. Two second fusion proteins comprise (i) an immunoglobulin x or X light chain (or portion thereof) and (ii) an extracellular domain of an MHC class IIa chain. The two first and the two second fusion proteins associate to form the MHC class II molecular complex. The extracellular domain of the MHC class II chain of each first fusion protein and the extracellular domain of the MHC class IIa chain of each second fusion protein form an MHC class II peptide binding cleft.

The immunoglobulin heavy chain can be the heavy chain of an IgM, IgD, IgG3, IgG1, IgG2P, IgG2a, IgG4, IgE, or IgA. In some embodiments, an IgG1 heavy chain is used to form divalent molecular complexes comprising two antigen binding clefts. Optionally, a variable region of the heavy chain can be included. IgM or IgA heavy chains can be used to provide pentavalent or tetravalent molecular complexes, respectively.

Fusion proteins of an MHC class II molecular complex can comprise a peptide linker inserted between an immunoglobulin chain and an extracellular domain of an MHC class II polypeptide. The length of the linker sequence can vary, depending upon the flexibility required to regulate the degree of antigen binding and receptor cross linking.

Immunoglobulin sequences in some embodiments are humanized monoclonal antibody sequences.”

The presently disclosed paramagnetic nano-aAPC also can have a costimulatory molecule bound thereto. Such costimulatory molecules can be referred to herein as a “Signal 2.” Such costimulatory molecules are generally a T cell affecting molecule, that is, a molecule that has a biological effect on a precursor T cell or on an antigen-specific T cell. Such biological effects include, for example, differentiation of a precursor T cell into a CTL, helper T cell (e.g., Th1, Th2), or regulatory T cell; and/or proliferation of T cells. Thus, T cell affecting molecules include T cell costimulatory molecules, adhesion molecules, T cell growth factors, and regulatory T cell inducer molecules. In some embodiments, an aAPC comprises at least one such ligand; optionally, an aAPC comprises at least two, three, or four such ligands.

In certain embodiments, signal 2 is a T cell costimulatory molecule. T cell costimulatory molecules contribute to the activation of antigen-specific T cells. Such molecules include, but are not limited to, molecules that specifically bind to CD28 (including antibodies), CD80 (B7-1), CD86 (B7-2), B7-H3, 4-1BB, 4-1BBL, CD27, CD30, CD134 (OX-40L), B7h (B7RP-1), CD40, LIGHT, antibodies that specifically bind to HVEM, antibodies that specifically bind to CD40L, antibodies that specifically bind to OX40, and antibodies that specifically bind to 4-1BB. In some embodiments, the costimulatory molecule (signal 2) is an antibody (e.g., a monoclonal antibody) or portion thereof, such as F(ab′)₂, Fab, scFv. or single chain antibody, or other antigen binding fragment. In some embodiments, the antibody is a humanized monoclonal antibody or portion thereof having antigen-binding activity, or is a fully human antibody or portion thereof having antigen-binding activity.

Adhesion molecules useful for nano-aAPC can be used to mediate adhesion of the nano-aAPC to a T cell or to a T cell precursor. Useful adhesion molecules include, for example, ICAM-1 and LFA-3.

In some embodiments, signal 1 is provided by peptide-HLA-A2 complexes, and signal 2 is provided by B7.1-Ig or anti-CD28. An exemplary anti-CD28 monoclonal antibody is 9.3 mAb (Tan et al., J. Exp. Med. 1993 177:165), which may be humanized in certain embodiments and/or conjugated to the bead as a fully intact antibody or an antigen-binding fragment thereof.

Some embodiments employ T cell growth factors, which affect proliferation and/or differentiation of T cells. Examples of T cell growth factors include cytokines (e.g., interleukins, interferons) and superantigens. If desired, cytokines can be present in molecular complexes comprising fusion proteins, or can be encapsulated by the aAPC.

Particularly useful cytokines include IL-2, IL-4, IL-7, IL-10, IL-12, IL-15, IL-21 gamma interferon, and CXCL10. Optionally, cytokines are provided solely by media components during expansion steps.

The nanoparticles can be made of any material, and materials can be appropriately selected for the desired magnetic property, and may comprise, for example, metals such as iron, nickel, cobalt, or alloy of rare earth metal. Paramagnetic materials also include magnesium, molybdenum, lithium, tantalum, and iron oxide. Paramagnetic beads suitable for enrichment of materials (including cells) are commercially available, and include iron dextran beads, such as dextran-coated iron oxide beads. In embodiments of the presently disclosed subject matter where magnetic properties are not required, nanoparticles can also be made of nonmetal or organic (e.g., polymeric) materials such as cellulose, ceramics, glass, nylon, polystyrene, rubber, plastic, or latex. In exemplary material for preparation of nanoparticles is poly(lactic-co-glycolic acid) (PLGA) and copolymers thereof, which may be employed in connection with these embodiments. Other materials including polymers and co-polymers that may be employed include those described in PCT/US2014/25889, which is hereby incorporated by reference in its entirety.

In some embodiments, the magnetic particles are biocompatible. This characteristic is particularly important in embodiments where the aAPC will be delivered to the patient in association with the enriched and expanded cells. For example, in some embodiments, the magnetic particles are biocompatible iron dextran paramagnetic beads.

In particular embodiments, the particle has a size (e.g., average diameter) of between about 100 nm to about 5000 nm, including about 100 nm, 200 nm, 300 nm, 400 nm, 500 nm, 600 nm, 700 nm, 800 nm, 900 nm, 1000 nm, 2000 nm, 3000 nm, 4000 nm, and 5000 nm. In some embodiments, the particle has a size of between about 100 nm to about 500 nm, including about 100 nm, 150 nm, 200 nm, 250 nm, 300 nm, 350 nm, 400 nm, 450 nm, and 500 nm. In particular embodiments, the particle has a size of about 300 nm. This size of magnetic nanoparticle affords the ability to use less expensive, lower power magnets, such as neodymium magnets associated with multi-well plates, to separate antigen-specific T cells associated with the magnetic nanoparticles. In previous embodiments, for example those disclosed in U.S. Pat. No. 10,098,939, which is incorporated herein by reference in its entirety, smaller superparamagnetic nanoparticles, e.g., 20 nm to about 200 nm, were used. These superparamagnetic nanoparticles of a smaller size required high gradient magnetic fields generated by specialized magnetic particle columns required to amplify the magnetic field strength.

Receptor-ligand interactions at the cell-nanoparticle interface are not well understood. Nanoparticle binding and cellular activation, however, are sensitive to membrane spatial organization, which is particularly important during T cell activation, and magnetic fields can be used to manipulate cluster-bound nanoparticles to enhance activation. See WO/2014/150132. For example, T cell activation induces a state of persistently enhanced nanoscale TCR clustering and nanoparticles are sensitive to this clustering in a way that larger particles are not. See WO/2014/150132, which is incorporated herein by reference in its entirety.

Furthermore, nanoparticle interactions with TCR clusters can be exploited to enhance receptor triggering. T cell activation is mediated by aggregation of signaling proteins, with “signaling clusters” hundreds of nanometers across, initially forming at the periphery of the T cell-APC contact site and migrating inward. As described herein, an external magnetic field can be used to enrich antigen-specific T cells (including rare naive cells) and to drive aggregation of magnetic nano-aAPC bound to TCR, resulting in aggregation of TCR clusters and enhanced activation of naive T cells. Magnetic fields can exert appropriately strong forces on paramagnetic particles, but are otherwise biologically inert, making them a powerful tool to control particle behavior. T cells bound to paramagnetic nano-aAPC are activated in the presence of an externally applied magnetic field. Nano-aAPC are themselves magnetized, and attracted to both the field source and to nearby nanoparticles in the field, inducing bead and thus TCR aggregation to boost aAPC-mediated activation. See WO/2014/150132.

Nano-aAPCs bind more TCR on and induce greater activation of previously activated compared to naive T cells. In addition, application of an external magnetic field induces nano-aAPC aggregation on naive cells, enhancing T cells proliferation both in vitro and following adoptive transfer in vivo. Importantly, in a melanoma adoptive immunotherapy model, T cells activated by nano-aAPC in a magnetic field mediate tumor rejection. Thus, the use of applied magnetic fields permits activation of naive T cell populations, which otherwise are poorly responsive to stimulation. This is an important feature of immunotherapy as naive T cells have been shown to be more effective than more differentiated subtypes for cancer immunotherapy, with higher proliferative capacity and greater ability to generate strong, long-term T cell responses. Thus, nano-aAPC can used for magnetic field enhanced activation of T cells to increase the yield and activity of antigen-specific T cells expanded from naive precursors, improving cellular therapy for example, patients with infectious diseases, cancer, or autoimmune diseases, or to provide prophylactic protection to immunosuppressed patients.

Molecules can be directly attached to nanoparticles by adsorption or by direct chemical bonding, including covalent bonding. See, Hermanson, BIOCONJUGATE TECHNIQUES, Academic Press, New York, 1996. A molecule itself can be directly activated with a variety of chemical functionalities, including nucleophilic groups, leaving groups, or electrophilic groups. Activating functional groups include alkyl and acyl halides, amines, sulfhydryls, aldehydes, unsaturated bonds, hydrazides, isocyanates, isothiocyanates, ketones, and other groups known to activate for chemical bonding. Alternatively, a molecule can be bound to a nanoparticle through the use of a small molecule-coupling reagent. Non-limiting examples of coupling reagents include carbodiimides, maleimides, n-hydroxysuccinimide esters, bischloroethylamines, bifunctional aldehydes such as glutaraldehyde, anhydrides and the like. In other embodiments, a molecule can be coupled to a nanoparticle through affinity binding such as a biotin-streptavidin linkage or coupling, as is well known in the art. For example, streptavidin can be bound to a nanoparticle by covalent or non-covalent attachment, and a biotinylated molecule can be synthesized using methods that are well known in the art.

If covalent binding to a nanoparticle is contemplated, the support can be coated with a polymer that contains one or more chemical moieties or functional groups that are available for covalent attachment to a suitable reactant, typically through a linker. For example, amino acid polymers can have groups, such as the s-amino group of lysine, available to couple a molecule covalently via appropriate linkers. This disclosure also contemplates placing a second coating on a nanoparticle to provide for these functional groups.

Activation chemistries can be used to allow the specific, stable attachment of molecules to the surface of nanoparticles. There are numerous methods that can be used to attach proteins to functional groups. For example, the common cross-linker glutaraldehyde can be used to attach protein amine groups to an aminated nanoparticle surface in a two-step process. The resultant linkage is hydrolytically stable. Other methods include use of cross-linkers containing n-hydrosuccinimido (NHS) esters which react with amines on proteins, cross-linkers containing active halogens that react with amine-, sulfhydryl-, or histidine-containing proteins, cross-linkers containing epoxides that react with amines or sulfhydryl groups, conjugation between maleimide groups and sulfhydryl groups, and the formation of protein aldehyde groups by periodate oxidation of pendant sugar moieties followed by reductive amination.

The ratio of particular ligands on the same nanoparticle can be varied to increase the effectiveness of the nanoparticle in antigen or costimulatory ligand presentation. For example, nanoparticles can be coupled with IILA-A2-Ig and anti-CD28 at a variety of ratios, such as about 30:1, about 25:1, about 20:1, about 15:1, about 10:1, about 5:1, about 3:1, about 2:1, about 1:1, about 0.5:1, about 0.3:1; about 0.2:1, about 0.1:1, or about 0.03:1. The total amount of protein coupled to the supports may be, for example, about 250 mg/mL, about 200 mg/mL, about 150 mg/mL, about 100 mg/mL, or about 50 mg/mL of particles. Because effector functions such as cytokine release and growth may have differing requirements for Signal 1 versus Signal 2 than T cell activation and differentiation, these functions can be determined separately.

The configuration of nanoparticles can vary from being irregular in shape to being spherical and/or from having an uneven or irregular surface to having a smooth surface. Non-spherical aAPCs are described in WO 2013/086500, which is hereby incorporated by reference in its entirety.

The aAPCs present antigen to T cells and thus can be used to both enrich for and expand antigen-specific T cells, including from naive T cells. The peptide antigens will be selected based on the desired therapy, for example, cancer, type of cancer, infectious disease, and the like. In some embodiments, the method is conducted to treat a cancer patient, and neoantigens specific to the patient are identified, and synthesized for loading aAPCs. In some embodiments, between three and ten neoantigens are identified through genetic analysis of the tumor (e.g., nucleic acid sequencing), followed by predictive bioinformatics. As shown herein, several antigens can be employed together (on separate aAPCs), with no loss of functionality in the method. In some embodiments, the antigens are natural, non-mutated, cancer antigens, of which many are known. This process for identifying antigens on a personalized basis is described in greater detail below.

A variety of antigens can be bound to antigen presenting complexes. The nature of the antigens depends on the type of antigen presenting complex that is used. For example, peptide antigens can be bound to MHC class I and class II peptide binding clefts. Non-classical MHC-like molecules can be used to present non-peptide antigens such as phospholipids, complex carbohydrates, and the like (e.g., bacterial membrane components such as mycolic acid and lipoarabinomannan). Any peptide capable of inducing an immune response can be bound to an antigen presenting complex. Antigenic peptides include tumor-associated antigens, autoantigens, alloantigens, and antigens of infectious agents.

The terms “cancer-specific antigen (CSA)” and “tumor-specific antigen (TSA)” are used interchangeably herein and refer to a protein, carbohydrate, or other molecule that is uniquely expressed by and/or displayed on cancer cells and is not expressed by or displayed on other cells in the body (e.g., normal healthy cells). In contrast, the terms “cancer-associated-antigen (CAA)” and “tumor-associated-antigen (TAA)” are used interchangeably herein and refer to a protein, carbohydrate, or other molecule that is not uniquely expressed by or displayed on a tumor cell and instead also is expressed on normal cells under certain conditions. Cancer-specific antigens and cancer-associated antigens are well known in the art. In some embodiments, the CSA or CAA comprises one or more antigenic cancer epitopes associated with a malignant cancer or tumor, a metastatic cancer or tumor, or a leukemia. A cancer “neoantigen” is a novel cancer-specific antigen that arises as a consequence of tumor-specific mutations (T.N. Schumacher and R.D. Schreiber, Science, 348(6230):69-74 (2015); and T.C. Wirth and F. Kuhnel, Front Immunol., 8: 1848 (2017)).

“Tumor-associated antigens” include unique tumor antigens expressed exclusively by the tumor from which they are derived, shared tumor antigens expressed in many tumors but not in normal adult tissues (oncofetal antigens), and tissue-specific antigens expressed also by the normal tissue from which the tumor arose. Tumor associated antigens can be, for example, embryonic antigens, antigens with abnormal post-translational modifications, differentiation antigens, products of mutated oncogenes or tumor suppressors, fusion proteins, or oncoviral proteins.

A variety of tumor-associated antigens are known in the art, and many of these are commercially available. Oncofetal and embryonic antigens include carcinoembryonic antigen and alpha-fetoprotein (usually only highly expressed in developing embryos but frequently highly expressed by tumors of the liver and colon, respectively), MAGE-1 and MAGE-3 (expressed in melanoma, breast cancer, and glioma), placental alkaline phosphatase sialyl-Lewis X (expressed in adenocarcinoma), CA-125 and CA-19 (expressed in gastrointestinal, hepatic, and gynecological tumors), TAG-72 (expressed in colorectal tumors), epithelial glycoprotein 2 (expressed in many carcinomas), pancreatic oncofetal antigen, 5T4 (expressed in gastriccarcinoma), alphafetoprotein receptor (expressed in multiple tumor types, particularly mammary tumors), and M2A (expressed in germ cell neoplasia).

Tumor-associated differentiation antigens include tyrosinase (expressed in melanoma) and particular surface immunoglobulins (expressed in lymphomas). Mutated oncogene or tumor-suppressor gene products include Ras and p53, both of which are expressed in many tumor types, Her-2/neu (expressed in breast and gynecological cancers), EGF-R, estrogen receptor, progesterone receptor, retinoblastoma gene product, myc (associated with lung cancer), ras, p53, nonmutant associated with breast tumors, MAGE-1, and MAGE-3 (associated with melanoma, lung, and other cancers). Fusion proteins include BCR-ABL, which is expressed in chromic myeloid leukemia. Oncoviral proteins include HPV type 16, E6, and E7, which are found in cervical carcinoma.

Tissue-specific antigens include melanotransferrin and MUC1 (expressed in pancreatic and breast cancers); CD10 (previously known as common acute lymphoblastic leukemia antigen, or CALLA) or surface immunoglobulin (expressed in B cell leukemias and lymphomas); the a chain of the IL-2 receptor, T cell receptor, CD45R, CD4⁺/CD8+(expressed in T cell leukemias and lymphomas); prostate specific antigen and prostatic acid-phosphatase (expressed in prostate carcinoma); GP 100, MelanA/Mart-1, tyrosinase, gp75/brown, BAGE, and S-100 (expressed in melanoma); cytokeratins (expressed in various carcinomas); and CD19, CD20, and CD37 (expressed in lymphoma).

Tumor-associated antigens also include altered glycolipid and glycoprotein antigens, such as neuraminic acid-containing glycosphingolipids (e.g., GM2 and GD2, expressed in melanomas and some brain tumors); blood group antigens, particularly T and sialylated Tn antigens, which can be aberrantly expressed in carcinomas; and mucins, such as CA-125 and CA-19-9 (expressed on ovarian carcinomas) or the underglycosylated MUC-1 (expressed on breast and pancreatic carcinomas).

“Antigens of infectious agents” include components of protozoa, bacteria, fungi (both unicellular and multicellular), viruses, prions, intracellular parasites, helminths, and other infectious agents that can induce an immune response.

Bacterial antigens include antigens of gram-positive cocci, gram positive bacilli, gram-negative bacteria, anaerobic bacteria, such as organisms of the families Actinomycetaceae, Bacillaceae, Bartonellaceae, Bordetellae, Captophagaceae, Corynebacteriaceae, Enterobacteriaceae, Legionellaceae, Micrococcaceae, Mycobacteriaceae, Nocardiaceae, Pasteurellaccae, Pseudomonadaceae, Spirochaetaceae, Vibrionaceae and organisms of the genera Acinetobacter, Brucella, Campylobacter, Erysipelothrix, Ewingella, Francisella, Gardnerella, Helicobacter, Levinea, Listeria, Streptobacillus and Tropheryma.

Antigens of protozoan infectious agents include antigens of malarial plasmodia, Leishmania species, Trypanosoma species and Schistosoma species. Fungal antigens include antigens of Aspergillus, Blastomyces, Candida, Coccidioides, Cryptococcus, Histoplasma, Paracoccicioides, Sporothrix, organisms of the order Mucorales, organisms inducing choromycosis and mycetoma and organisms of the genera Trichophyton, Microsporum, Epidermophyton, and Malassezia.

Viral peptide antigens include, but are not limited to, those of adenovirus, herpes simplex virus. papilloma virus, respiratory syncytial virus, poxviruses, HIV, influenza viruses, and CMV. Particularly useful viral peptide antigens include HIV proteins such as HIV gag proteins (including, but not limited to, membrane anchoring (MA) protein, core capsid (CA) protein and nucleocapsid (NC) protein), HIV polymerase, influenza virus matrix (M) protein and influenza virus nucleocapsid (NP) protein, hepatitis B surface antigen (HBsAg), hepatitis B core protein (HBcAg), hepatitis e protein (HBeAg), hepatitis B DNA polymerase, hepatitis C antigens, and the like.

Antigens, including antigenic peptides, can be bound to an antigen binding cleft of an antigen presenting complex either actively or passively, as described in U.S. Pat. No. 6,268,411, which is hereby incorporated by reference in its entirety. Optionally, an antigenic peptide can be covalently bound to a peptide binding cleft.

If desired, a peptide tether can be used to link an antigenic peptide to a peptide binding cleft. For example, crystallographic analyses of multiple class I MHC molecules indicate that the amino terminus of 02M is very close, approximately 20.5 Angstroms away, from the carboxyl terminus of an antigenic peptide resident in the MHC peptide binding cleft. Thus, using a relatively short linker sequence, approximately 13 amino acids in length, one can tether a peptide to the amino terminus of p2M. If the sequence is appropriate, that peptide will bind to the MHC binding groove (see U.S. Pat. No. 6,268,411).

Antigen-specific T cells which are bound to the aAPCs can be separated from cells which are not bound using magnetic enrichment, or other cell sorting or capture technique.

Other processes that can be used for this purpose include flow cytometry and other chromatographic means (e.g., involving immobilization of the antigen-presenting complex or other ligand described herein). In one embodiment antigen-specific T cells are isolated (or enriched) by incubation with beads, for example, antigen-presenting complex/anti-CD28-conjugated paramagnetic beads (such as DYNABEADS®), for a time period sufficient for positive selection of the desired antigen-specific T cells.

In some embodiments, a population of T cells can be substantially depleted of previously active T cells using, e.g., an antibody to CD44, leaving a population enriched for naive T cells. Binding nano-aAPCs to this population would not substantially activate the naive T cells, but would permit their purification.

In still other embodiments, ligands that target NK cells, NKT cells, or B cells (or other immune effector cells), can be incorporated into a paramagnetic nanoparticle, and used to magnetically enrich for these cell populations, optionally with expansion in culture as described below. Additional immune effector cell ligands are described in PCT/US2014/25889, which is hereby incorporated by reference in its entirety.

Without wishing to be bound by theory, removal of unwanted cells may reduce competition for cytokines and growth signals, remove suppressive cells, or may simply provide more physical space for expansion of the cells of interest.

Enriched T cells are then expanded in culture within the proximity of a magnet to produce a magnetic field, which enhances T cell receptor clustering of aAPC bound cells. Cultures can be stimulated for variable amounts of time (e.g., about 0.5, 2, 6, 12, 36, 48, or 72 hours as well as continuous stimulation) with nano-aAPC. The effect of stimulation time in highly enriched antigen-specific T cell cultures can be assessed. Antigen-specific T cell can be placed back in culture and analyzed for cell growth, proliferation rates, various effector functions, and the like, as is known in the art. Such conditions may vary depending on the antigen-specific T cell response desired. In some embodiments, T cells are expanded in culture from about 2 days to about 3 weeks, or in some embodiments, about 5 days to about 2 weeks, or about 5 days to about 10 days. In some embodiments, the T cells are expanded in culture for about 1 week, after which time a second enrichment and expansion step is optionally performed. In some embodiments, 2, 3, 4, or 5 enrichment and expansion rounds are performed.

After the one or more rounds of enrichment and expansion, the antigen-specific T cell component of the sample will be at least about 1% of the cells, or in some embodiments, at least about 5%, at least about 10%, at least about 15%, or at least about 20%, or at least about 25% of the cells in the sample. Further, these T cells generally display an activated state. From the original sample isolated from the patient, the antigen-specific T cells in various embodiments are expanded from about 100-fold to about 10,000 fold, such as at least about 1000-fold, at least about 2000-fold, at least about 3.000 fold, at least about 4,000-fold, or at least about 5,000-fold in various embodiments. After the one or more rounds of enrichment and expansion, at least about 10⁶, or at least about 10⁷, or at least about 10⁸, or at least about 10⁹ antigen-specific T cells are obtained.

The effect of nano-aAPC on expansion, activation and differentiation of T cell precursors can be assayed in any number of ways known to those of skill in the art. A rapid determination of function can be achieved using a proliferation assay, by determining the increase of CTL, helper T cells, or regulatory T cells in a culture by detecting markers specific to each type of T cell. Such markers are known in the art. CTL can be detected by assaying for cytokine production or for cytolytic activity using chromium release assays.

In addition to generating antigen-specific T cells with appropriate effector functions, another parameter for antigen-specific T cell efficacy is expression of homing receptors that allow the T cells to traffic to sites of pathology (Sallusto et al., Nature 401, 708-12, 1999; Lanzavecchia & Sallusto, Science 290, 92-97, 2000).

For example, effector CTL efficacy has been linked to the following phenotype of homing receptors, CD62L+, CD45RO+, and CCR7−. Thus, a nano-aAPC-induced and/or expanded CTL population can be characterized for expression of these homing receptors. Homing receptor expression is a complex trait linked to initial stimulation conditions. Presumably, this is controlled both by the costimulatory complexes as well as cytokine milieu. One important cytokine that has been implicated is IL-12 (Salio et al., 2001). As discussed below, nano-aAPC offer the potential to vary individually separate components (e.g., T cell effector molecules and antigen presenting complexes) to optimize biological outcome parameters. Optionally, cytokines such as IL-12 can be included in the initial induction cultures to affect honing receptor profiles in an antigen-specific T cell population.

Optionally, a cell population comprising antigen-specific T cells can continue to be incubated with either the same nano-aAPC or a second nano-aAPC for a period of time sufficient to form a second cell population comprising an increased number of antigen-specific T cells relative to the number of antigen-specific T cells in the first cell population. Typically, such incubations are carried out for 3-21 days, preferably 7-10 days.

Suitable incubation conditions (culture medium, temperature, etc.) include those used to culture T cells or T cell precursors, as well as those known in the art for inducing formation of antigen-specific T cells using DC or artificial antigen presenting cells. See, e.g., Latouche & Sadelain, Nature Biotechno. 18, 405-09, April 2000; Levine et al., J. Immunol. 159, 5921-30, 1997; Maus et al., Nature Biotechnol. 20, 143-48, February 2002. See also the specific examples, below.

To assess the magnitude of a proliferative signal, antigen-specific T cell populations can be labeled with CFSE and analyzed for the rate and number of cell divisions. T cells can be labeled with CFSE after one-two rounds of stimulation with nano-aAPC to which an antigen is bound. At that point, antigen-specific T cells should represent 2-10% of the total cell population. The antigen-specific T cells can be detected using antigen-specific staining so that the rate and number of divisions of antigen-specific T cells can be followed by CFSE loss. At varying times (for example, 12, 24, 36. 48, and 72 hours) after stimulation, the cells can be analyzed for both antigen presenting complex staining and CFSE. Stimulation with nano-aAPC to which an antigen has not been bound can be used to determine baseline levels of proliferation. Optionally, proliferation can be detected by monitoring incorporation of 3H-thymidine, as is known in the art.

### C. Methods for Personalized Medicine

In some embodiments, the presently disclosed subject matter provides methods for personalized medicine, including cancer immunotherapy. The methods are accomplished using the aAPCs to identify antigens to which the patient will respond, followed by administration of the appropriate peptide-loaded aAPC to the patient, or followed by enrichment and expansion of the antigen specific T cells ex vivo.

Genome-wide sequencing has dramatically altered our understanding of cancer biology. Sequencing of cancers has yielded important data regarding the molecular processes involved in the development of many human cancers. Driving mutations have been identified in key genes involved in pathways regulating three main cellular processes (1) cell fate, (2) cell survival and (3) genome maintenance. Vogelstein et al., Science 339, 1546-58 (2013).

Genome-wide sequencing also has the potential to revolutionize our approach to cancer immunotherapy. Sequencing data can provide information about both shared as well as personalized targets for cancer immunotherapy. In principle, mutant proteins are foreign to the immune system and are putative tumor-specific antigens. Indeed, sequencing efforts have defined hundred if not thousands of potentially relevant immune targets. Limited studies have shown that T cell responses against these neo-epitopes can be found in cancer patients or induced by cancer vaccines. However, the frequency of such responses against a particular cancer and the extent to which such responses are shared between patients are not well known. One of the main reasons for our limited understanding of tumor-specific immune responses is that current approaches for validating potential immunologically relevant targets are cumbersome and time consuming.

Thus, in some embodiments, the presently disclosed subject matter provides a high-throughput platform-based approach for detection of T cell responses against neo-antigens in cancer. This approach uses the aAPC platform described herein for the detection of even low-frequency T cell responses against cancer antigens. Understanding the frequency and between-person variability of such responses would have important implications for the design of cancer vaccines and personalized cancer immunotherapy.

Although central tolerance abrogates T cell responses against self-proteins, oncogenic mutations induce neo-epitopes against which T cell responses can form. Mutation catalogues derived from whole exome sequencing provide a starting point for identifying such neo-epitopes. Using HLA binding prediction algorithms (Srivastava, PLoS One 4, e6094 (2009), it has been predicted that each cancer can have up 7-10 neo-epitopes. A similar approach estimated hundreds of tumor neo-epitopes. Such algorithms, however, may have low accuracy in predicting T cell responses, and only 10% of predicted HLA-binding epitopes are expected to bind in the context of HLA (Lundegaard C, Immunology 130, 309-18 (2010)). Thus, predicted epitopes must be validated for the existence of T cell responses against those potential neo-epitopes.

In certain embodiments, the nano-aAPC system is used to screen for neo-epitopes that induce a T cell response in a variety of cancers, or in a particular patient's cancer.

Cancers may be genetically analyzed, for example, by whole exome-sequencing. For example, of a panel of 24 advanced adenocarcinomas, an average of about 50 mutations per tumor were identified. Of approximately 20,000 genes analyzed, 1327 had at least one mutation, and 148 had two or more mutations. 974 missense mutations were identified, with a small additional number of deletions and insertions.

A list of candidate peptides can be generated from overlapping nine amino acid windows in mutated proteins. All nine-AA windows that contain a mutated amino acid, and 2 non-mutated “controls” from each protein will be selected. These candidate peptides will be assessed computationally for MHC binding using a consensus of MHC binding prediction algorithms, including NetMHC and stabilized matrix method (SMM). Nano-aAPC and MHC binding algorithms have been developed primarily for HLA-A2 allele. The sensitivity cut-off of the consensus prediction can be adjusted until a tractable number of mutation containing peptides (approximately 500) and non-mutated control peptides (approximately 50) are identified.

A peptide library is then synthesized. MHC (e.g., A2) bearing aAPC are deposited in multi well plates and passively loaded with peptide. CD8 T cells may be isolated from PBMC of both A2 positive healthy donors and A2 positive pancreatic cancers patients (or other cancer or disease described herein). Subsequently, the isolated T cells are incubated with the loaded aAPCs in the plates for the enrichment step. Following the incubation, the plates are placed on a magnetic field and the supernatant containing irrelevant T cells not bound to the aAPCs is removed. The remaining T cells that are bound to the aAPCs will be cultured and allowed to expand for 7 to 21 days. Antigen specific expansion is assessed by re-stimulation with aAPC and intracellular IFNγ fluorescent staining.

In some embodiments, a patient's T cells are screened against an array or library of nanoAPCs, and the results are used for diagnostic or prognostic purposes. For example, the number and identity of T cell anti-tumor responses against mutated proteins, overexpressed proteins, and/or other tumor-associated antigens can be used as a biomarker to stratify risk. For example, the number of such T cell responses may be inversely proportionate to the risk of disease progression or risk of resistance or non-responsiveness to chemotherapy. In other embodiments, the patient's T cells are screened against an array or library of nano-APCs, and the presence of T cells responses, or the number or intensity of these T cells responses identifies that the patient has a sub-clinical tumor, and/or provides an initial understanding of the tumor biology.

In some embodiments, a patient or subject's T cells are screened against an array or library of paramagnetic aAPCs, each presenting a different candidate peptide antigen. This screen can provide a wealth of information concerning the subject or patient's T cell repertoire, and the results are useful for diagnostic or prognostic purposes. For example, the number and identity of T cell anti-tumor responses against mutated proteins, overexpressed proteins, and/or other tumor-associated antigens can be used as a biomarker to stratify risk, to monitor efficacy of immunotherapy, or predict outcome of immunotherapy treatment.

Further, the number or intensity of such T cell responses may be inversely proportionate to the risk of disease progression or may be predictive of resistance or non-responsiveness to chemotherapy. In other embodiments, a subject's or patient's T cells are screened against an array or library of nano-APCs each presenting a candidate peptide antigen, and the presence of T cells responses, or the number or intensity of these T cells responses, provides information concerning the health of the patient, for example, by identifying autoimmune disease, or identifying that the patient has a sub-clinical tumor. In these embodiments, the process not only identifies a potential disease state, but provides an initial understanding of the disease biology.

### C.1 Methods for Treating a Disease, Disorder, or Condition

In some embodiments, the presently disclosed subject matter provides methods for treating a disease, disorder, or condition through immunotherapy in which detection, enrichment and/or expansion of antigen-specific immune cells ex vivo is therapeutically or diagnostically desirable. Accordingly, the presently disclosed subject matter is generally applicable for detecting, enriching and/or expanding antigen-specific T cells, including cytotoxic T lymphocytes (CTLs), helper T cells, and regulatory T cells.

Antigen-specific T cells obtained using nano-aAPC, can be administered to patients by any appropriate routes, including intravenous administration, intra-arterial administration, subcutaneous administration, intradermal administration, intralymphatic administration, and intratumoral administration. Patients include both human and veterinary patients.

Antigen-specific regulatory T cells can be used to achieve an immunosuppressive effect, for example, to treat or prevent graft versus host disease in transplant patients, or to treat or prevent autoimmune diseases, such as those listed above, or allergies. Uses of regulatory T cells are disclosed, for example, in US 2003/0049696, US 2002/0090724, US 2002/0090357, US 2002/0034500, and US 2003/0064067, which are hereby incorporated by reference in their entireties.

Antigen-specific T cells prepared according to these methods can be administered to patients in doses ranging from about 5-10×10⁶ CTL/kg of body weight (approximately 7×10⁸ CTL/treatment) up to about 3.3×10⁹ CTL/kg of body weight (approximately 6×10⁹ CTL/treatment) (Walter et al., New England Journal of Medicine 333, 1038-44, 1995; Yee et al., J Exp Med 192, 1637-44, 2000). In other embodiments, patients can receive about 10³, about 5×10³, about 10⁴, about 5×10⁴, about 10⁵, about 5×10⁵, about 10⁶, about 5×10⁶, about 10⁷, about 5×10⁷, about 108, about 5×10⁸, about 10⁹, about 5×10⁹, or about 10¹⁰ cells per dose administered intravenously. In still other embodiments, patients can receive intranodal injections of, e.g., about 8×10⁶ or about 12×10⁶ cells in a 200 L bolus. Doses of nano-APC that are administered with cells include about 10³, about 5×10³, about 10⁴, about 5×10⁴, about 10⁵, about 5×10⁵, about 10⁶, about 5×10⁶, about 10⁶, about 5×10⁷, about 10⁸, about 5×10⁸, about 10⁹, about 5×10⁹, or about 10¹⁰ nano-aAPC per dose.

In an exemplary embodiment, the enrichment and expansion process is performed repeatedly on the same sample derived from a patient. A population of T cells is enriched and activated on Day 0, followed by a suitable period of time (e.g., about 3-20 days) in culture. Subsequently, nano-aAPC can be used to again enrich and expand against the antigen of interest, further increasing population purity and providing additional stimulus for further T cell expansion. The mixture of nano-aAPC and enriched T cells may subsequently again be cultured in vitro for an appropriate period of time, or immediately re-infused into a patient for further expansion and therapeutic effect in vivo. Enrichment and expansion can be repeated any number of times until the desired expansion is achieved.

In some embodiments, a cocktail of nano-aAPC, each against a different antigen, can be used at once to enrich and expand antigen T cells against multiple antigens simultaneously. In this embodiment, a number of different nano-aAPC batches, each bearing a different MHC-peptide, would be combined and used to simultaneously enrich T cells against each of the antigens of interest. The resulting T cell pool would be enriched and activated against each of these antigens, and responses against multiple antigens could thus be cultured simultaneously. These antigens could be related to a single therapeutic intervention; for example, multiple antigens present on a single tumor.

In some embodiments, the patient receives immunotherapy with one or more checkpoint inhibitors, prior to receiving the antigen-specific T cells by adoptive transfer, or prior to direct administration of aAPCs bearing neoantigens identified in vitro through genetic analysis of the patient's tumor. In various embodiments, the checkpoint inhibitor(s) target one or more of CTLA-4 or PD-1/PD-Li, which may include antibodies against such targets, such as monoclonal antibodies, or portions thereof, or humanized or fully human versions thereof. In some embodiments, the checkpoint inhibitor therapy comprises ipilimumab or Keytruda (pembrolizumab).

In some embodiments, the patient receives about 1 to 5 rounds of adoptive immunotherapy (e.g., one, two, three, four or five rounds). In some embodiments, each administration of adoptive immunotherapy is conducted simultaneously with, or after (e.g., from about 1 day to about 1 week after), a round of checkpoint inhibitor therapy. In some embodiments, adoptive immunotherapy is provided about 1 day, about 2 days, or about 3 days after checkpoint inhibitor therapy.

In still other embodiments, adoptive transfer or direct infusion of nano-aAPCs to the patient comprises, as a ligand on the bead, a ligand that targets one or more of CTLA-4 or PD-1/PD-Li. In these embodiments, the method can avoid certain side effects of administering soluble checkpoint inhibitor therapy.

### C.1.1 Methods for Treating Cancer

In some embodiments, the disease, disorder, or condition is a cancer. In particular embodiments, the cancer is a solid tumor or a hematological malignancy. The enrichment and expansion of antigen-specific CTLs ex vivo for adoptive transfer to a patient provides for a robust anti-tumor immune response.

Cancers that can be treated or evaluated according to the presently disclosed methods include cancers that historically illicit poor immune responses or have a high rate of recurrence. Exemplary cancers include various types of solid tumors, including carcinomas, sarcomas, and lymphomas. In various embodiments the cancer is melanoma (including metastatic melanoma), colon cancer, duodenal cancer, prostate cancer, breast cancer, ovarian cancer, ductal cancer, hepatic cancer, pancreatic cancer, renal cancer, endometrial cancer, testicular cancer, stomach cancer, dysplastic oral mucosa, polyposis, head and neck cancer, invasive oral cancer, non-small cell lung carcinoma, small-cell lung cancer, mesothelioma, transitional and squamous cell urinary carcinoma, brain cancer, neuroblastoma, and glioma.

In some embodiments, the cancer is a hematological malignancy, such as chronic myelogenous leukemia, childhood acute leukemia, non-Hodgkin's lymphomas, chronic lymphocytic leukemia, malignant cutaneous T-cells, mycosis fungoids, non-MF cutaneous T-cell lymphoma, lymphomatoid papulosis, T-cell rich cutaneous lymphoid hyperplasia, and discoid lupus erythematosus.

In various embodiments, the cancer is stage I, stage II, stage III, or stage IV. In some embodiments, the cancer is metastatic and/or recurrent. In some embodiments, the cancer is preclinical, and is detected in the screening system described herein (e.g., colon cancer, pancreatic cancer, or other cancer that is difficult to detect early).

### C.1.2 Method for Treating an Infectious Disease

In other embodiments, the presently disclosed subject matter includes a method for treating an infectious disease. The infectious disease may be one in which enrichment and expansion of antigen-specific immune cells (such as CD8⁺ or CD4⁺ T cells) ex vivo for adoptive transfer to the patient could enhance or provide for a productive immune response. Infectious diseases that can be treated include those caused by bacteria, viruses, prions, fungi, parasites, helminths, and the like. Such diseases include AIDS, hepatitis, CMV infection, and post-transplant lymphoproliferative disorder (PTLD).

CMV, for example, is the most common viral pathogen found in organ transplant patients and is a major cause of morbidity and mortality in patients undergoing bone marrow or peripheral blood stem cell transplants. This is due to the immunocompromised status of these patients, which permits reactivation of latent virus in seropositive patients or opportunistic infection in seronegative individuals. A useful alternative to these treatments is a prophylactic immunotherapeutic regimen involving the generation of vims-specific CTL derived from the patient or from an appropriate donor before initiation of the transplant procedure. PTLD occurs in a significant fraction of transplant patients and results from Epstein-Barr virus (EBV) infection. EBV infection is believed to be present in approximately 90% of the adult population in the United States. Active viral replication and infection is kept in check by the immune system. but, as in cases of CMV, individuals immunocompromised by transplantation therapies lose the controlling T cell populations, which permits viral reactivation. This represents a serious impediment to transplant protocols. EBV may also be involved in tumor promotion in a variety of hematological and non-hematological cancers.

Other viral pathogens potentially treated by the presently disclosed methods include, but are not limited to adenovirus, herpes simplex virus, papilloma virus, respiratory syncytial virus, poxviruses, HIV, influenza viruses, and COVID-19.

### C.1.3 Method for Treating an Autoimmune Disease

In some embodiments, the patient has an autoimmune disease, in which enrichment and expansion of regulatory T cells (e.g., CD4⁺, CD25+, Foxp3+) ex vivo for adoptive transfer to the patient could dampen the deleterious immune response. Autoimmune diseases that can be treated include systemic lupus erythematosus, rheumatoid arthritis, type I diabetes, multiple sclerosis, Crohn's disease, ulcerative colitis, psoriasis, myasthenia gravis, Goodpasture's syndrome, Graves' disease, pemphigus vulgaris, Addison's disease, dermatitis herpetiformis, celiac disease, and Hashimoto's thyroiditis. In some embodiments, the patient is suspected of having an autoimmune disease or immune condition (such as those described in the preceding sentence), and the evaluation of T cell responses against a library of paramagnetic nano-aAPCs as described herein, is useful for identifying or confirming the immune condition.

### D. Reagents/Kits

In other embodiments, the presently disclosed subject matter provides a kit comprising the presently disclosed nano-aAPCs together with components for performing the enrichment and expansion process. Suitable containers for the presently disclosed paramagnetic nanoparticles include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). Optionally, one or more different antigens can be bound to the paramagnetic nanoparticles or can be supplied separately. Kits may comprise, alternatively or in addition, one or more multi-well plates or culture plates for T cells. In some embodiments, kits comprise a sealed container comprising paramagnetic nanoparticles, a magnet, and optionally test tubes and/or solution or buffers for performing magnetic enrichment.

A kit can further comprise a second container comprising a pharmaceutically acceptable buffer, such as phosphate-buffered saline, Ringer's solution, or dextrose solution. It can also contain other materials useful to an end user, including other buffers, diluents, filters, needles, and syringes.

Kits also may contain reagents for assessing the extent and efficacy of antigen-specific T cell activation or expansion, such as antibodies against specific marker proteins, MHC class I or class II molecular complexes, TCR molecular complexes, anticlonotypic antibodies, and the like.

A kit can also comprise a package insert containing written instructions for methods of inducing antigen-specific T cells, expanding antigen-specific T cells, using paramagnetic nanoparticles in the kit in various protocols. The package insert can be an unapproved draft package insert or can be a package insert approved by the Food and Drug Administration (FDA) or other regulatory body.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, and the like.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of. in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1. and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Nanoparticle-Based Modulation of CD4⁺ T Cell Effector and Helper Functions Enhances Adoptive Immunotherapy

### 1.1 Overview

Helper (CD4⁺) T cells perform direct therapeutic functions and augment responses of cells, such as cytotoxic (CD8⁺) T cells, against a wide variety of diseases and pathogens. Nevertheless, inefficient synthetic technologies for expansion of antigen-specific CD4⁺ T cells hinders consistency and scalability of CD4⁺ T cell-based therapies, and complicates mechanistic studies. Here we describe a nanoparticle platform for ex vivo CD4⁺ T cell culture that mimics antigen presenting cells (APC) through display of major histocompatibility class II (MHC II) molecules. When combined with soluble co-stimulation signals, MHC II artificial APCs (aAPC) expand cognate murine CD4⁺ T cells, including rare endogenous subsets, to induce potent effector functions in vitro and in vivo. Moreover, MHC II aAPCs provide help signals that enhance antitumor function of aAPC-activated CD8⁺ T cells in a mouse tumor model. Lastly, human leukocyte antigen class II-based aAPCs expand rare subsets of functional, antigen-specific human CD4⁺ T cells. Overall, MHC II aAPCs provide a promising approach for harnessing targeted CD4⁺ T cell responses.

### 1.2 Background

Clinical successes of adoptive cell transfer (ACT) therapies across a wide range of hematologic malignancies, Grubb et al., 2013; Porter et al., 2011, and solid tumors, Tran et al., 2014; Hunder et al., 2008, have propelled T cell therapies to the forefront of treatment options for a variety of cancers and other diseases. Despite their promise, some of the largest hurdles these therapies face in moving toward widespread translation are the associated time, costs, and complexities of ex vivo T cell expansion, Isser et al., 2021, as well as the variability of the resulting clinical products. A range of approaches has been developed for ex vivo expansion of tumor-specific T cells, including polyclonal T cell stimulation with plate- or bead-bound anti-CD3 (αCD3) antibodies, or antigen-specific T cell stimulation with peptide-pulsed autologous antigen presenting cells (APCs). To simultaneously address the lack of specificity of αCD3 stimulation, as well as the manufacturing challenges and variability of donor-derived APCs, biomimetic artificial APCs (aAPCs) that include MHC proteins and co-stimulatory molecules have been produced. Oelke et al., 2003. Thus far, these synthetic platforms have focused almost exclusively on CD8⁺ T cells, whereas little progress has been made for CD4⁺ targeted technologies.

CD4⁺ T cells serve several critical functions in the antitumor immune response, including recognizing neoantigens that result from tumor-specific mutations, Kreiter et al., 2015; Alspach et al., 2019, recruiting and activating innate immune cells, Mumberg et al., 1999, Hung et al., 1998, Perez-diez et al., 2016, directly lysing MHC II positive tumor cells, Quezada et al., 2010, and relaying indispensable “help” signals to CD8⁺ T cells to enhance their antitumor function and memory formation. Borst et al., 2018. A simplified system that modulates these functions could pave the way toward scalable, consistent CD4⁺ T cell or “helped” CD8⁺ T cell cancer therapies, while also providing mechanistic insight into CD4⁺ T cell tumor biology.

Herein, we describe a platform for antigen-specific CD4⁺ T cell expansion, consisting of iron-dextran nanoparticles coated with MHC II and co-stimulatory proteins. These MHC II aAPCs lead to expansion of cognate murine CD4⁺ T cells that display high levels of effector cytokine production and demonstrate robust lytic capacity in vitro and in vivo. MHC II aAPCs also relay help signals from CD4⁺ T cells to tumor-specific CD8⁺ T cells, which, in turn, enhance CD8⁺ T cell cytokine production, memory formation, and in vitro and in vivo antitumor activity. Lastly, murine MHC II and human counterpart HLA II aAPCs can expand rare subsets of endogenous murine and human CD4⁺ T cells. Together. this work highlights a variety of applications of nanoparticle technologies for enrichment, expansion, and modulation of CD4⁺ T cell effector and helper functions.

### 1.3 Results

1.3.1 MHC II aAPCs stimulate functional antigen-specific murine CD4⁺ T cells

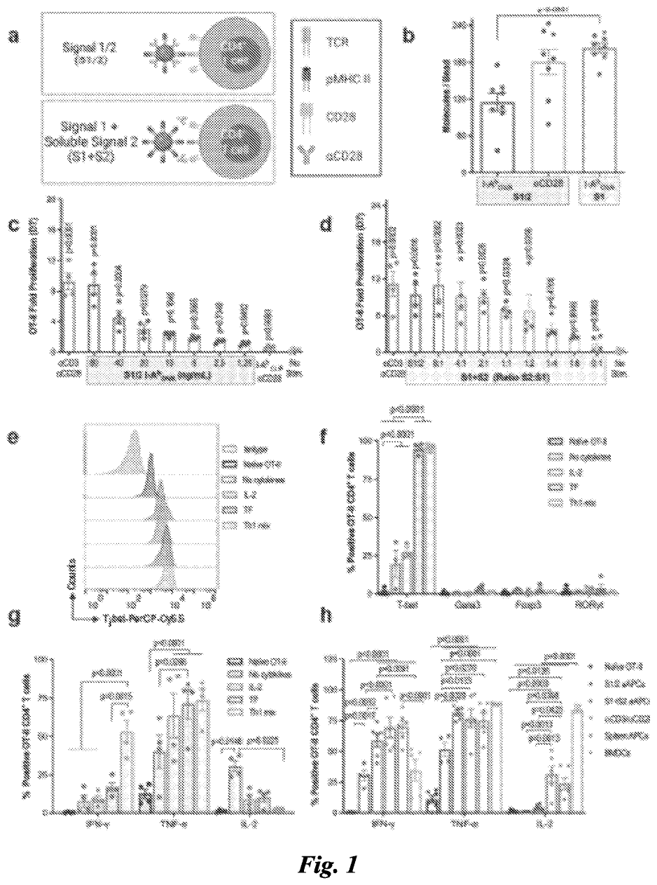

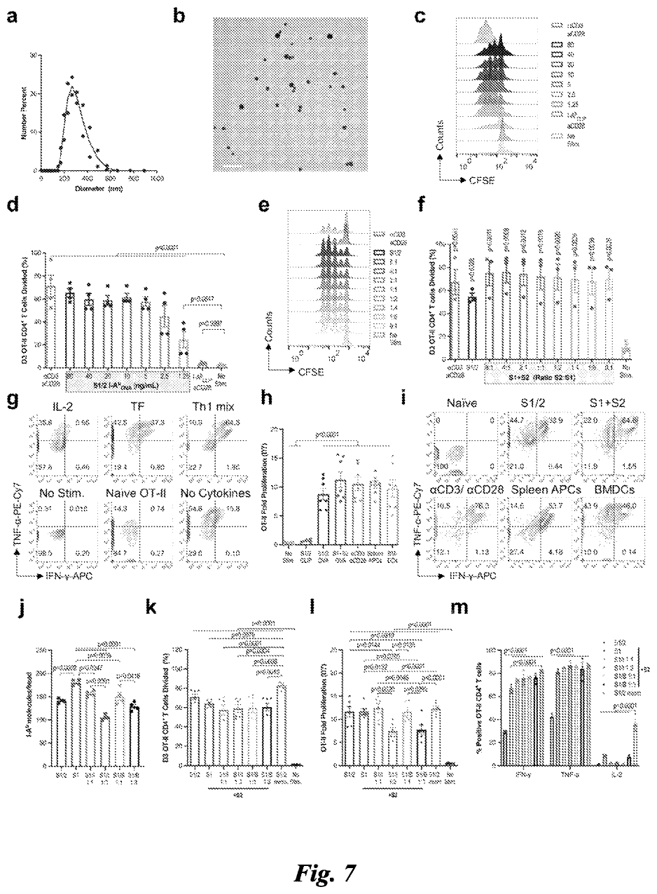

T cells require two signals to become activated: T cell receptor (TCR) stimulation, known as signal 1 (S1) through cognate peptide-loaded MHC (pMHC) interactions, and co-stimulation, termed signal 2 (S2), most commonly through the CD28 receptor. TCR99 pMHC interactions tend to be lower affinity for CD4⁺ T cells than for CD8⁺ T cells. Sugata et al., 2021. Based on this premise, we formulated two aAPC designs for ex vivo activation of antigen-specific murine CD4⁺ T cells: one that, similar to traditional MHC I aAPCs6, co-presents MHC II I-A^{b} proteins and anti-CD28 (αCD28) antibodies (S1/2) and a second that presents only I-A^{b} proteins, with addition of soluble αCD28 (S1+S2), to maximize MHC II valency on aAPCs (FIG. 1a). To synthesize the aAPCs, signals were conjugated to 200-nm iron oxide nanoparticles, a size which corresponds to the pre-formed TCR clusters found on naive T cells, Lillemeier et al., 2010, and which we have previously shown is optimal for CD8⁺ T cell engagement. Hickey et al., 2017. Post fabrication, the aAPCs were approximately 300 nm in size (FIG. 7a-b), with around 100 I-A^{b} molecules per S1/2 bead and 200 I-A^{b} molecules per S1 bead (FIG. 1b).

Through titration of the S1/2 aAPCs into culture with TCR transgenic OT-II ovalbumin (OVA) specific CD4⁺ T cells, we found that a concentration of 80 ng/mL I-A^{b}loaded with the OVA₃₂₉₋₃₃₇ peptide (I-A_{OVA}^{b}) led to similar percentage of T cells dividing at day 3 (FIG. 7c-d) and fold proliferation at day 7 (FIG. 1c) compared to control αCD3/αCD28 microbeads. The stimulation was antigen-specific, as I-A^{b} aAPCs loaded with an irrelevant CLIP₈₇₋₁₀₁ peptide (I-A_{CLIP}^{b}) did not induce OT-II proliferation (FIG. 1c, FIG. 7c-d). For S1 aAPCs, increasing the amount of soluble S2 added into culture did not impact the percentage of cells dividing at day 3 (FIG. 7e-f) but did increase fold proliferation at day 7 to levels similar to αCD3/αCD28 microbeads and S1/2 aAPCs (FIG. 1d). As certain CD4⁺ T cell subsets can either promote or inhibit antitumor responses, Tay et al., 2021 (e.g., Th1 versus regulatory T cells), we next analyzed CD4⁺ T cell polarization, by activating OT-II CD4⁺ T cells with MHC II aAPCs in the presence of various cytokine mixes. In comparison to treatment with interleukin-2 (IL-2) only and no cytokines, a Th1 mix (IL-2, IL-12p70, and IFN-γ) led to higher T-bet expression (FIG. 1e124f) and IFN-7 production (FIG. 1g, FIG. 7g), hallmark transcription factors and cytokines of the Th1 lineage, respectively. Interestingly, a standard T cell growth factor (TF) cytokine cocktail for CD8⁺ T cell culture, Oelke et al., 2000, led to a similar increase in T-bet levels but not IFN-γ production.

We also compared the impact of different types of T cell stimulation on OT-II cell proliferation and function when cultured in the Th1 mix. We found that optimal doses of S1/2 and S1+S2 aAPCs led to equivalent proliferation as αCD3/αCD28 microbeads, OT-II splenocytes pulsed with OVA₃₂₃₋₃₃₉ peptide, or bone marrow derived dendritic cells (BMDCs) (FIG. 7h). Additionally, S1+S2 aAPC stimulation led to IFN-γ production equivalent to peptide-pulsed splenocytes or αCD3/αCD28 microbeads, whereas cells cultured with S1/2 aAPCs or BMDCs produced less IFN-γ (FIG. 1h, FIG. 7i).

To assess the impact of signal density on aAPC-mediated OT-II proliferation and function, we mixed I-A_{OVA}^{b} proteins at 1:1 and 1:3 molar ratios with isotype antibodies (S1/I) or bovine serum albumin (S1/B), detecting lower conjugation of I-A^{b} at higher ratios of these additional proteins (FIG. 7j). We found that at equivalent doses of S 1(80 ng/mL), aAPCs with lower S1 density led to similar percentages of OT-II cells dividing at day 3 (FIG. 7k), but lower overall proliferation at day 7 compared to higher density S1 or S1/2 aAPCs (FIG. 7I). That said, the density of S1 did not impact OT-II function at day 7 (FIG. 7m). We performed similar analyses using 4.5-μm S1/2 aAPCs that more closely mimic the size of endogenous APCs, observing no significant differences in OT-II proliferation or IFN-γ and TNF-α secretion compared to nano-aAPCs, but higher frequencies of IL-2 producing cells (FIG. 7k-m).

Together, these results demonstrate robust expansion of functional, antigen-specific CD4⁺ T cell is achieved by both S1/2 and S1+S2 MHC II aAPCs and that the extent of expansion is directly dependent upon S1 density.

1.3.2 MHC II aAPCs Expand Rare Murine CD4⁺ T Cell Subsets

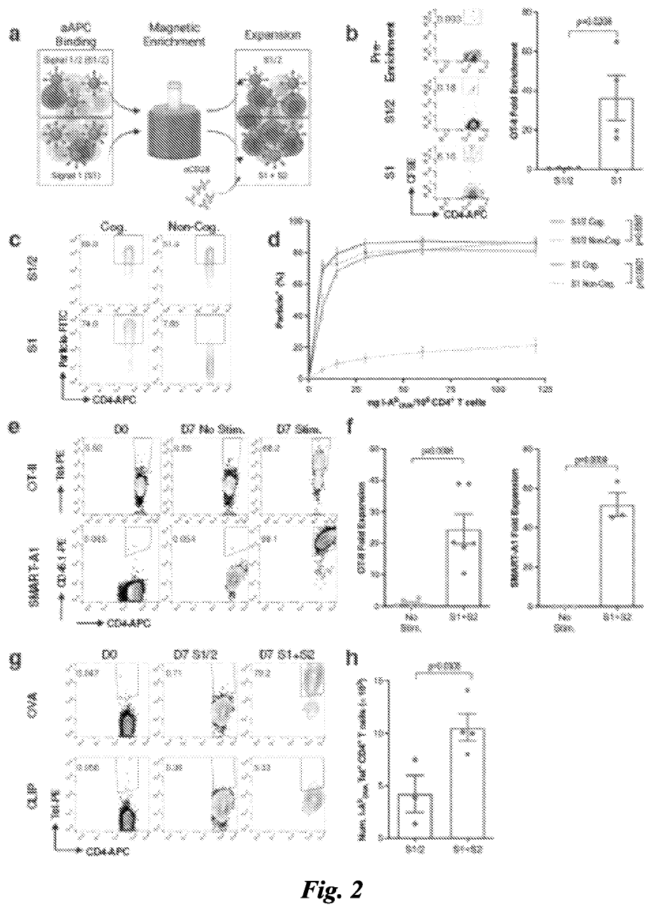

To explore whether MHC II aAPCs could be used to expand rare antigen-specific CD4⁺ T cells, we employed an analogous approach to our previous work with murine, Perica et al., 2015; Hickey et al., 2018, and human, Ichikawa et al., 2020, CD8⁺ T cells, following a three-step process that includes aAPCs binding to T cells, magnetic enrichment of aAPC-bound T cells, and expansion of the enriched T cell product (FIG. 2a). For S1 aAPCs, excess soluble S2 was added to the enriched product to facilitate T cell expansion. To optimize the enrichment and expansion system, we diluted CFSE labelled OT-II CD4⁺ T cells at a ratio of 1:1000 into a background of C57BL/6 (B6) CD4⁺ T cells. Mimicking MHC II tetramer binding protocols, Cameron et al., 2001, we incubated the mixed cell population with aAPCs at 37° C. for 2 hours, followed by magnetic enrichment of aAPC-bound cells using a 96 well plate magnet compatible with our 300 nm aAPCs. Hickey et al., 2020. Immediately post enrichment, we found that S1 aAPCs led to significantly higher fold enrichment than S1/2 aAPCs (FIG. 2b). To understand why this was, we tracked aAPC binding to cognate OT-II CD4⁺ T cells compared to non-cognate B6 CD4⁺ T cells. S1 aAPCs bound with significantly greater specificity to cognate CD4⁺ T cells across a range of doses (FIG. 2c-d).

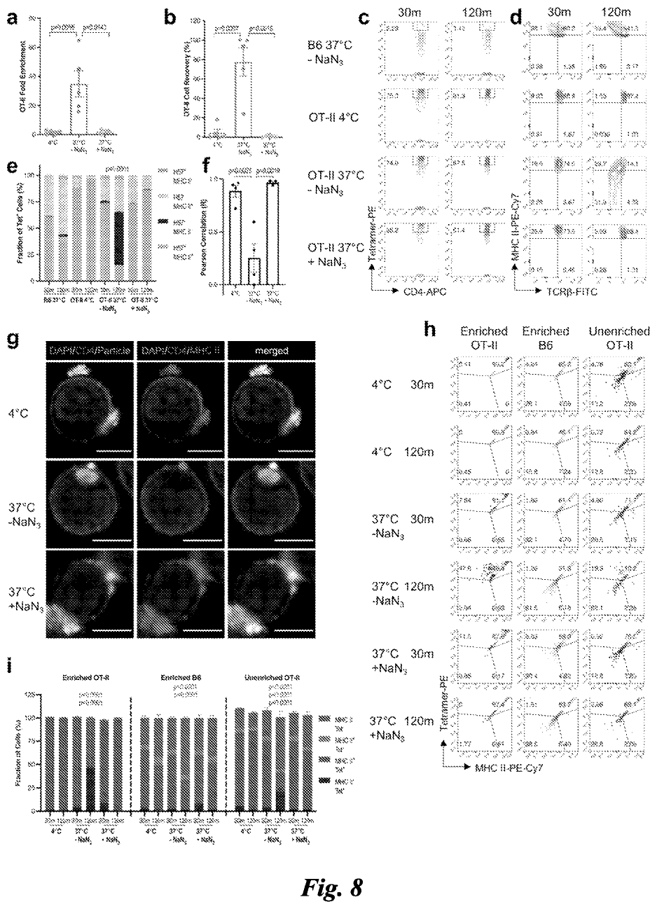

The temperature of incubation, as well as active cellular processes, both affected the enrichment and recovery of diluted OT-II cells, as binding at 4° C. or metabolic inhibition with sodium azide (NaN₃) each impaired the enrichment process (FIG. 8a-b). To understand these findings, we tracked nanoparticle internalization over time by incubating cells with 200-nm particles conjugated with PE-labelled I-A_{OVA}^{b} tetramers. Particles remaining on the surface of cells were subsequently detected with an anti-MHC II antibody. Interestingly, OT-II cells remained positive for the tetramer-labelled particles regardless of incubation time, temperature, or metabolic inhibition (FIG. 8c); however, within the tetramer positive cell populations, two hours of incubation at 37° C. in the absence of NaN₃ led to a significant reduction in cells staining positive for MHC II, indicating internalization of the particles (FIG. 8d-e). Loss of MHC II staining coincided with downregulation of TCRs, suggesting the particle internalization was TCR-mediated; indeed, non-cognate B6 CD4⁺ T cells that were incubated with particles in the same manner non-specifically bound to but did not internalize aAPCs (FIG. 8d-e).

We confirmed aAPC internalization through confocal microscopy, observing a significant drop in the spatial correlation between particle and MHC II fluorescence at 37° C. in the absence of NaN₃ (FIG. 8f-g). To more closely assess the impact of aAPC internalization on enrichment of rare CD4⁺ T cell populations, we performed an enrichment study as above with CFSE labelled OT-II CD4⁺ T cells, analyzing the surface binding versus internalization patterns of cognate CFSE⁺ OT-II and non-cognate CFSE⁻ B6 CD4⁺ T cells. We found that a two-hour incubation period at 37° C. in the absence of NaN₃ allowed for enrichment of OT-1I cells with either surface-bound or internalized aAPCs (FIG. 8h-i, left). In contrast, all other incubation conditions only enriched OT-II cells with surface-bound aAPCs. Moreover, enriched B6 cells from this incubation condition showed minimal particle internalization, despite non-specifically binding to the aAPCs (FIG. 8h-i, middle), demonstrating the antigen193 specificity of internalization, even in mixed samples.

Finally, unlike other conditions where OT-II CD4⁺ T cells were lost in the enrichment process even when bound at high levels with aAPCs, the majority of unenriched OT-II CD4⁺ T cells from samples incubated at 37° C. without metabolic inhibition, were tetramer negative (FIG. 8h-i, right), suggesting that aAPC interaction is more likely to lead to cell recovery in this condition.

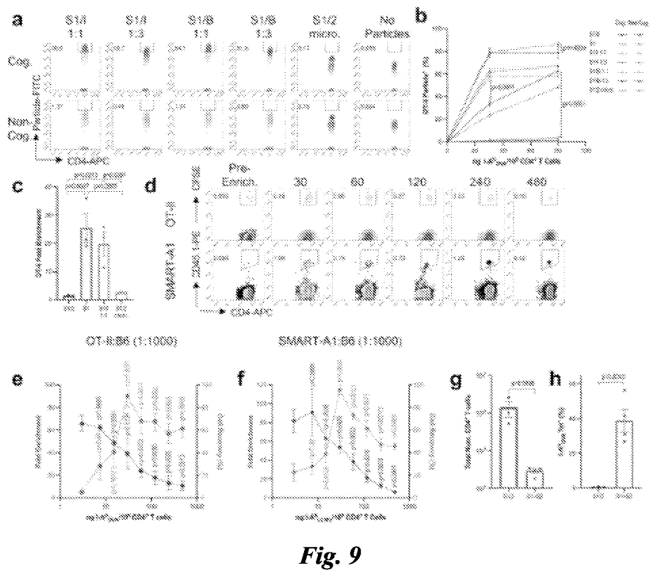

To examine whether poor specific binding and enrichment of OT-II CD4⁺ T cells with S1/2 aAPCs was due to lower TCR-pMHC avidity compared to S1 aAPCs or non-specific CD28/αCD28 interactions, we examined the binding of lower density S1/I and S1/B aAPCs (FIG. 7j) to cognate OT-II and non-cognate B6 CD4⁺ T cells. We found that while lower S1 density aAPCs bound less effectively to OT-II cells, their specific binding still remained significantly higher than their non-specific binding, unlike S1/2 aAPCs (FIG. 9a-b). Indeed S1/I aAPCs yielded similar fold enrichment of diluted OT-IT cells, as S1 aAPCs (FIG. 9c), despite having a slightly lower density of I-A^{b} (FIG. 7j). In contrast, 4.5- m S1/2 aAPCs bound poorly to, and failed to enrich, cognate cells (FIG. 9a-c).

The dose of aAPCs also affected the efficiency of enrichment and recovery of TCR transgenic OT-II and SMART-A1 lymphocytic choriomeningitis virus glycoprotein (I-Ab LCMV GP61-80) specific CD4⁺ T cells, with optimal cell enrichments and recoveries being achieved at 30 ng I-A^{b}/10⁶ CD4⁺ T cells (FIG. 9d-f). Using S1+S2 aAPCs with this optimized enrichment protocol, we observed 30-50-fold expansion of OT-II and SMART-A1 CD4⁺ T cells after 7 days (FIG. 2e-f). Likewise, the optimized protocol allowed us to expand a nearly 80% specific population of endogenous I-Abov_{A} specific CD4⁺ T cells from a naive B6 background in 7 days (FIG. 2g-h, FIG. 9g-h). Based on estimated precursor frequencies of I-A_{OVA}^{b} CD4⁺ T cells in B6 mice, Moon et al., 2007, this corresponds to approximately 1000-fold expansion. In contrast, S1/2 aAPCs yielded a higher total number of CD4⁺ T cells at day 7, but both the percentage and number of antigen-specific CD4⁺ T cells were significantly reduced (FIG. 2g-h, FIG. 9g-h), illustrating that separation of S1 and S2 can dramatically increase the frequency of rare antigen-specific CD4⁺ T cell populations.

1.3.3 MHC II aAPCs Promote CD4⁺ T Cell Cytotoxicity

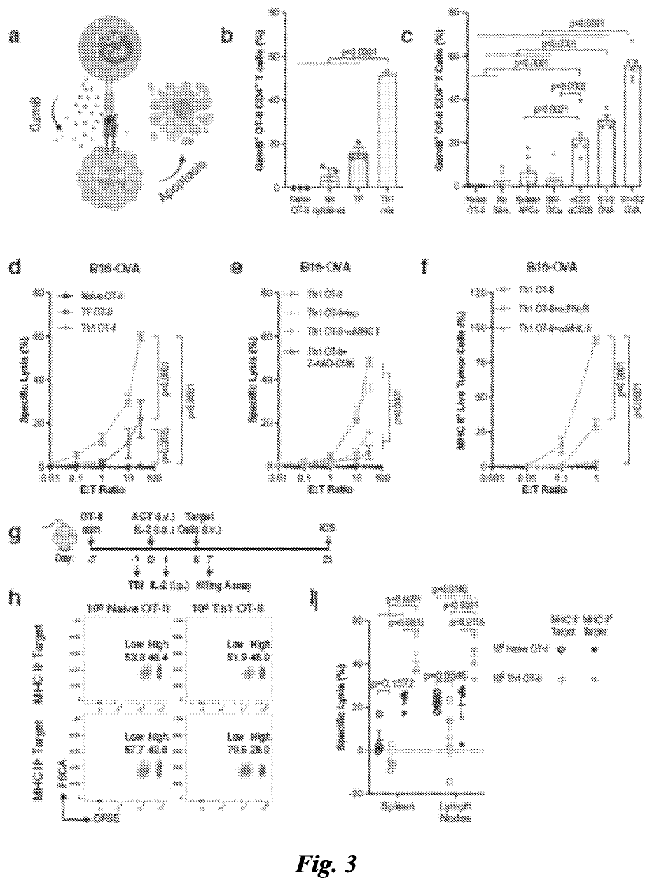

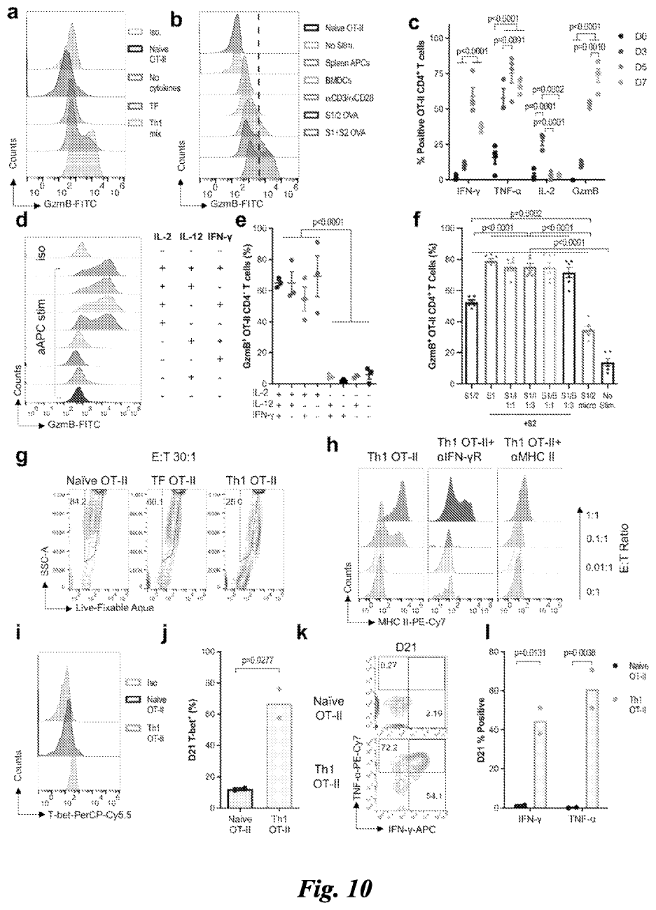

There have been published reports of CD4⁺ T cell acquisition of cytotoxic functions, Quezada et al., 2010, Oh et al., 2020, Cachot et al., 2021, Melenhorst et al., 2022, in various disease states but there is no consistent method for producing them or studying them ex vivo. To assess the impact of MHC II aAPCs on CD4⁺ T cell cytotoxicity, we monitored production of the serine protease Granzyme B (GzmB) and associated lytic capacity of aAPC activated CD4⁺ T cells (FIG. 3a). We found that induction of CD4⁺ T cell cytotoxicity was sensitive to both TCR engagement and the cytokine milieu. We observed a dramatic increase in GzmB levels when aAPC-activated CD4⁺ T cells were cultured in Th1 media compared to TF or cytokine-free media (FIG. 3b, FIG. 10a). In Th1 media, S1+S2 stimulation induced significantly higher levels of GzmB production than αCD3/αCD28 stimulation or the use of splenocytes or BMDCs pulsed with peptide (FIG. 3c, FIG. 10b). GzmB production increased over the course of S1+S2 stimulation (FIG. 10c) and was specifically dependent on the presence of IL-2 in the Th1 mix (FIG. 10d-e). Additionally, we found that GzmB induction depended on soluble, as opposed to conjugated S2, and nano-, as opposed to micro-particles, but did not depend on S1 density (FIG. 10f). Consequently, S1+52 stimulated OT-II CD4⁺ T cells were able to lyse B16-OVA tumor cells in vitro when cultured in Th1 media (FIG. 3d, FIG. 10g) in a GzmB and MHC II dependent manner (FIG. 3e). To elucidate how OT-II recognition of B16-OVA tumor cells was occurring, given that most tumors do not constitutively express MHC II, we monitored MHC II expression on live B16-OVA cells after co-culture with S1+52 aAPC activated OT-II cells, finding that the CD4⁺ T cells induce MHC II expression on B16-OVA in an IFN-γ dependent manner (FIG. 3f, FIG. 10h).

We next assessed the in vivo functional activity of S1+S2 aAPC activated OT-II CD4⁺ T cells by examining their lytic capacity and cytokine production 7 and 21 days post adoptive transfer into CD45.1 B6 mice (FIG. 3g). At day 7, aAPC activated cells specifically lysed OVA₃₂₃₋₃₃9 pulsed target cells in an MHC II-restricted manner (FIG. 3h-i). Activated cells persisted through day 21, remaining T-bet positive (FIG. 10i-j) and continuing to secrete IFN-γ and TNF-α (FIG. 10k-1). Collectively, in vitro and in vivo cytotoxicity studies revealed that aAPCs can activate lytic programs in CD4⁺ T cells.

1.3.4 MHC II aAPCs Modulate CD4⁺ T Cell Helper Function

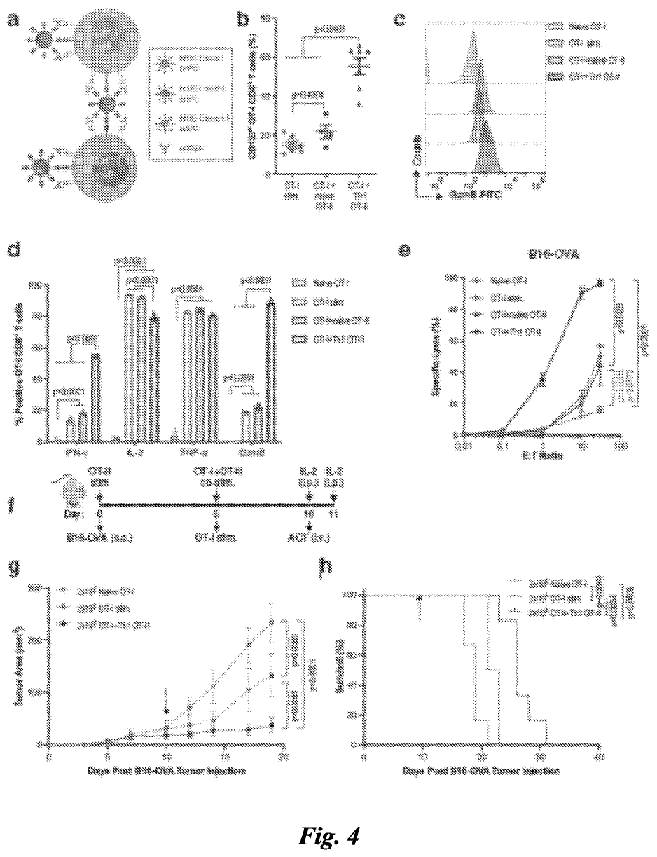

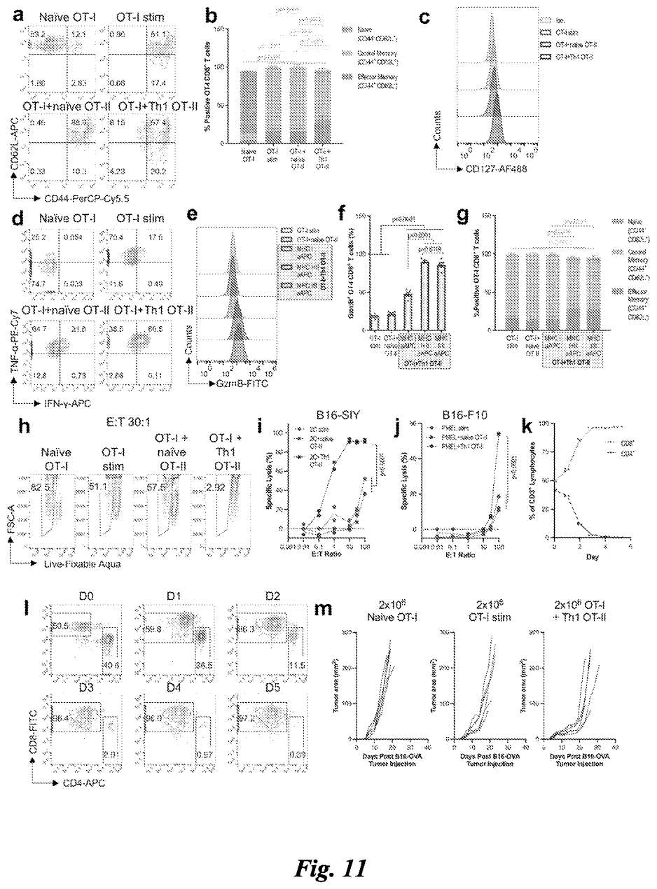

One objective in developing MHC II aAPCs was to produce a scalable approach for generating CD4⁺ T cells that could enhance the memory formation, function, and cytotoxicity of tumor-specific CD8⁺ T cells. Borst et al., 2018. To do so. CD4⁺ and CD8⁺ T cells were co-activated either with separate MHC I and MHC II aAPCs (MHC 1+II) or with a novel aAPC made by coupling nanoparticles with both MHC I and MHC II (MHC 1/II) (FIG. 4a). In all cases, αCD28 was delivered in solution. We first assessed the impact of CD4⁺ and CD8⁺ co-activation on CD8⁺ T cell memory formation and function by co-culturing TCR transgenic K^{b}OVA₂₅₇₋₂₆₄ specific OT-I CD8⁺ T cells (OT-I) at a 1:1 ratio with either naive OT-II CD4⁺ T cells or OT-II CD4⁺ T cells activated with S1+52 aAPCs (Th1 OT-II). We found that co-stimulation of OT-I with Th1 OT-II cells using separate MHC I+II aAPCs led to an increase in effector memory CD8⁺ T cells that also expressed significantly higher levels of IL-7 receptor-alpha (IL-7Ra or CD127), a marker associated with long lasting memory T cells (FIG. 4b, FIG. 11a-c). The addition of Th1 OT-II cells also increased OT-I production of GzmB (FIG. 4c-d) and IFN-γ (FIG. 4d, FIG. 11d). This effect was dependent on restimulation of the Th1 OT-II cells with either MHC 1/11 or MHC I+II aAPCs (FIG. 11e-g). Furthermore, co-culture with Th1 OT-II cells significantly boosted the in vitro cytotoxicity of TCR Transgenic OT-I, 2C Kb SIY, and PMEL Db gp100₂₅₋₃₃ specific CD8⁺ T cells against B16- OVA (FIG. 4e, FIG. 11h), B16-SIY (FIG. 11i), and B16-F10 tumor cells (FIG. 11j), respectively. To determine whether CD4⁺ T cell help led to superior in vivo antitumor efficacy of CD8⁺ T cell therapies, we used an adoptive transfer model of pre-established murine melanoma (FIG. 4f). In this model, B6 mice were injected subcutaneously with B16-OVA tumor cells and then treated 10 days later with either naive OT-I CD8⁺ T cells, aAPC activated OT-I CD8⁺ T cells, or OT-I CD8⁺ T cells co-activated with Th1 OT-II CD4⁺ T cells using MHC I+II aAPCs. By the day of treatment, all of the cells in the co-culture condition were CD8⁺, allowing direct comparisons of the antitumor function of CD8⁺ T cells across these three conditions (FIG. 11k-1). We found that treatment with OT-I CD8⁺ T cells that had been co-cultured with CD4⁺ T cells resulted in significantly improved B16-OVA antitumor control (FIG. 4g, FIG. 11m) and enhanced survival (FIG. 4h) compared to both naive or aAPC-activated OT-I CD8⁺ T cells. No tumors in treated mice fully regressed, potentially due to antigenic escape, a common limitation of this model. Kaluza et al., 2012. Hence, both in vitro assays and in vivo disease models corroborated the beneficial role of aAPC-stimulated CD4⁺ T cells in boosting CD8′ T cell function.

1.3.5 aAPC Mediated T Cell Help is Driven by Soluble Factors and Extends to Endogenous CD8⁺ T Cells

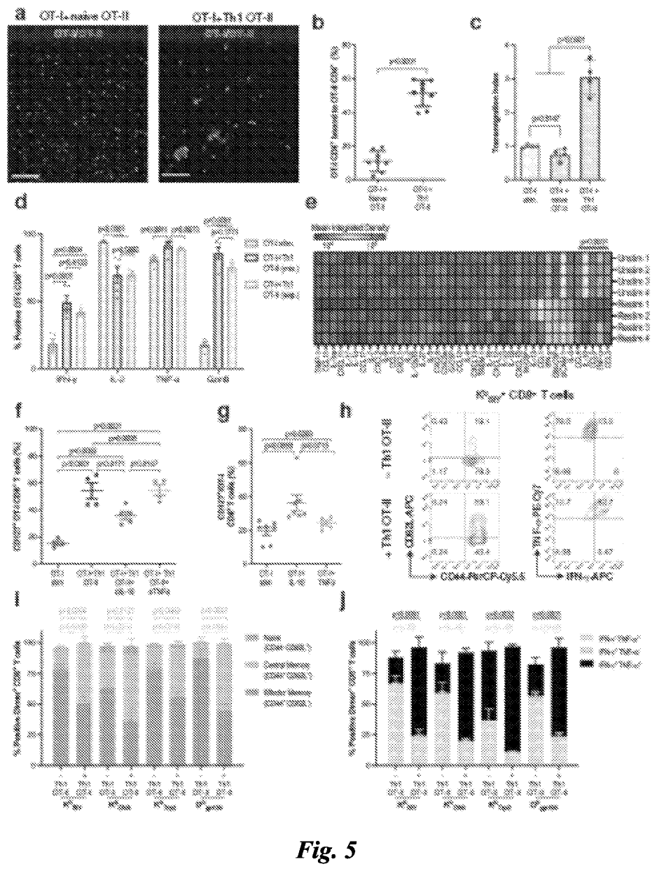

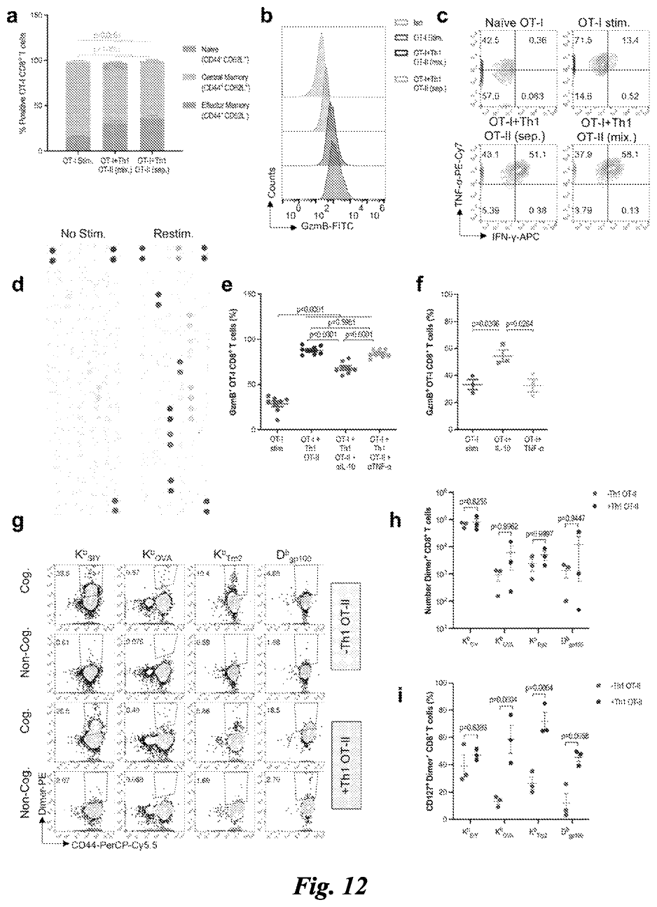

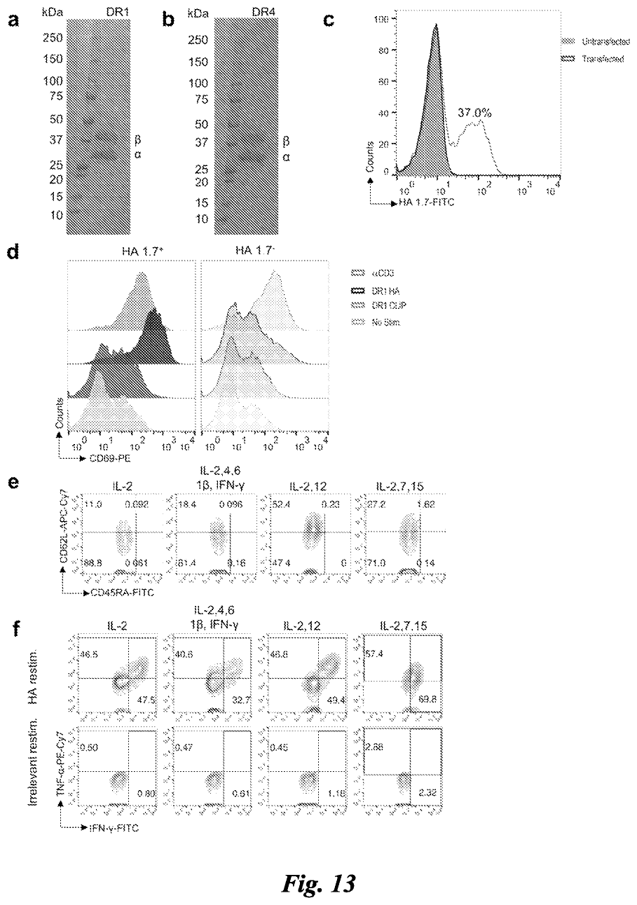

To better understand the mechanisms underlying bolstered activity of CD8⁺ T cells co-cultured with CD4⁺ T cells, we performed epifluorescent imaging of OT-I cells mixed with naive or Th1 OT-II CD4⁺ T cells. After 24 hours of co-incubation in the presence of MHC I/II aAPCs, OT-I CD8⁺ T cells had significantly more cell-cell interactions with Th1 OT-II than with naive OT-II cells (FIG. 5a-b). Accordingly, Th1 OT-1I cells induced significantly greater transmigration of OT-I than naive OT-II cells (FIG. 5c). To assess whether this enhanced cell-cell interaction was complementary to or a requirement for improving OT-I function, we performed transwell assays, wherein OT-I and Th1 OT-II cells were either mixed together in the same well or separated by a 0.4-μm membrane. Interestingly, separation of OT-I and Th1 OT-II did not affect CD8⁺ memory skewing (FIG. 12a) or function (FIG. 5d, FIG. 12b-c), suggesting that MHC II aAPC mediated CD4⁺ help occurred through soluble factors. Based on these results, we analyzed the supernatants of Th1 OT-11 cells using a cytokine protein array (FIG. 5e, FIG. 12d). The results indicated that the most highly abundant cytokines and chemokines were IL-10, TNF-α, CCL3, CCL4, and CCL5. Since chemokines CCL3, CCL4, and CCL5 primarily affect T cell migration, we focused on analyzing the impact of IL-10 and TNF-α. We found through blocking IL-10 and TNF-α in co-culture experiments and adding exogenous IL-10 and TNF-α to OT-I only cultures, that IL-10 specifically impacts OT-I GzmB production (FIG. 12e-f) and CD127 expression (FIG. 5f-g). Since help signals were observed to be delivered in solution, we next assessed how they would impact the memory phenotype and function of endogenous antigen-specific CD8⁺ T cells. To answer this question, we followed our existing protocol, Hickey et al., 2020, for enrichment and expansion of CD8⁺ T cells from naive B6 mice, with or without Th1 OT-II CD4⁺ T cells added to the enriched fractions. We found that the addition of CD4⁺ T cells did not significantly alter the number of antigen-specific CD8⁺ T cells on day 7 (FIG. 12g-h), but enhanced the central memory phenotype of antigen-specific CD8⁺ T cells (FIG. 5h-i), their IFN-γ production (FIG. 5h,j), and CD127 expression (FIG. 12i).

1.3.6 HLA II aAPCs Stimulate Functional Antigen-Specific Human CD4⁺ T Cells

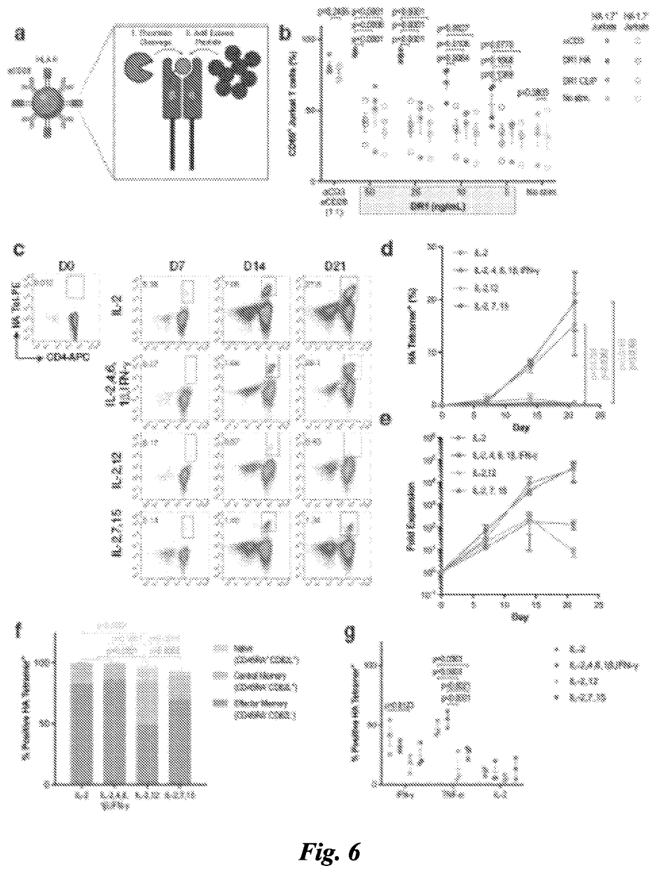

To establish whether the MHC II aAPC technology could be translated for human CD4⁺ T cell culture, we designed and expressed HLA class II monomers following a previously described system. Day et al., 2003 (FIG. 13a-b). We then covalently attached these HLA molecules and αCD28 proteins to iron dextran particles which could then be adapted to a range of target antigens through thrombin cleavage and peptide exchange (FIG. 6a). To assess the function and specificity of peptide-exchanged aAPCs, we exchanged HLA DR1 aAPCs overnight with the hemagglutinin HA₃₀₆₋₃₁₈ peptide and then monitored their ability to activate Jurkat cells transfected overnight with the HA₃₀₆₋₃₁₈-recognizing HA 1.7 TCR (FIG. 13c). DR1 aAPCs loaded with the cognate HA peptide (DR1 HA) upregulated CD69, a T cell activation marker, specifically on the HA 1.7 positive Jurkat cells; Moreover, unlike αCD3 based stimulation, which also activated HA 1.7 negative Jurkat cells, DR1 HA aAPCs were specific for the HA 1.7 expressing Jurkats (FIG. 6b, FIG. 13d).

We next assessed whether we could expand HA specific CD4⁺ T cells from healthy DR4 donors, using DR4/αCD28 aAPCs. We compared expansion in four different cytokine mixes: IL-2 expansion media; IL-2, IL-4, IL-6, IL-β, and IFN-γ human CD8⁺ culture media; Ichikawa et al., 2020; IL-2 and IL-12 Th1 skewing media; and IL-2, IL-7, and IL-15 memory skewing media. We found that both IL-2 media and IL-2,4,6,1β, and IFN-γ media resulted in robust expansion of HA specific CD4⁺ T cells from nearly undetectable precursor frequencies (FIG. 6c) to approximately 30% of the cell mixture (FIG. 6d), leading to nearly 100,000-fold expansion over the course of 21 days (FIG. 6e). Unlike with murine CD4⁺ T cells, this antigen-specific expansion was achieved without needing to separate S1 and S2. In contrast, IL-2 and 12 media and IL-2, 7, and 15 media only yielded modest expansions that declined after day 14. The resulting phenotype of the HA-specific CD4⁺ T cells from IL-2 or IL-2, 4, 6, 1$, and IFN-γ media was predominantly effector memory-like (FIG. 6f, FIG. 13e) and approximately 30-40% of the cells were IFN-γ and TNF-α positive after antigen-specific restimulation (FIG. 6g, FIG. 13f). Taken together, these results demonstrate that in the optimized cytokine milieu, HLA II aAPCs can expand rare antigen-specific human CD4⁺ T cells from endogenous repertoires.

### 1.4 Discussion

Synthetic technologies for ex vivo expansion of T cells have continued to evolve over the past several decades to incorporate the breadth of biophysical and chemical cues that have been shown to affect T cell function. Isser et al., 2021. These tools have thus far focused primarily on polyclonal T cell stimulation or expansion of antigen-specific CD8⁺ T cells, Hickey et al., 2018; Cachot et al.,2021; Cheung et al., 2018; Rhodes et al., 2021; Fadel et al., 2014. However, for many disease or pathogen-specific applications, CD8⁺ T cells may play a less dominant role than other T cell subsets, particularly CD4⁺ T cells. Even in cancer, where CD8⁺ T cells are central to the therapeutic immune response, the antitumor function of these cells may be suboptimal without the addition of CD4⁺ T cell help at both the priming, Zander et al., 2019, and effector, Alspach et al., 2019, stages. pMHC II-coated beads have been developed for in vivo induction of regulatory T cells in autoimmunity. Clemente-Casares et al., 2016; Singha et al., 2017. However, technologies that harness effector or helper roles of CD4⁺ T cells have yet to be explored.

To address these limitations, here we developed the MHC II aAPC, a nanoparticle platform for ex vivo expansion of antigen-specific murine and human CD4⁺ T cells. The platform confers several advantages over existing approaches to CD4⁺ T cell expansion such as αCD3/αCD28 microparticles and peptide-pulsed autologous dendritic cells (DCs). αCD3/αCD28 microparticles provide non-specific stimulation that can result in potential expansion of irrelevant or even pathogenic T cells, Ichikawa et al., 2020; Maus et al., 2002, presenting a hurdle for expansion of rare subsets of antigen-specific T cells. Autologous DCs provide antigen-specific stimulation; however, they require complex manufacturing steps, their availability is limited, Wöfl et al., 2014, and the level and composition of signals they present to T cells are minimally controllable, which is of particular concern for cancer patients whose DCs are often dysfunctional, Gigante et al., 2009; Satthaporn et al., 2004, or even immunosuppressive. Wculek et al., 2020.

Here we showed that MHC II aAPCs could be used off the shelf to activate murine and human CD4⁺ T cells at levels similar to non-specific αCD3/αCD28 stimulation, while maintaining specificity for cognate CD4⁺ T cells. Furthermore, MHC II aAPCs were able to specifically expand initially undetectable antigen-specific murine and human CD4⁺ T cells from endogenous T cell repertoires. MHC II aAPCs could additionally be used in conjunction with existing synthetic platforms for ex vivo CD8⁺ T cell activation to relay crucial help signals from CD4⁺ T cells to a wide range of CD8⁺ T cells. These help signals, in turn, boosted the memory formation, IFN-γ production, cytotoxicity, and in vivo antitumor control of antigen-specific CD8⁺ T cells. Thus, the MHC II aAPC presents a streamlined approach for ex vivo generation of personalized CD4⁺ T cell and the provision of helper signals to CD8⁺ T cell therapies.

In addition to the clinical applications of the MHC II aAPC, it also provides a bottom-up approach for exploring CD4⁺ T cell biology. For instance, here we show that MHC II aAPC stimulation results in generation of cytotoxic CD4⁺ T cells, a phenotype which, thus far, has been observed primarily in vivo. Quezada et al., 2010; Oh et al., 2020; Cachot et al., 2021; Melenhorst et al., 2022. While confirming the importance of IL-2 in this process, Sledzinska et al., 2020, we also observed that differentiation of CD4⁺ T cells into cytotoxic T lymphocytes (CTL) occurred after stimulation with artificial and not endogenous APCs. Further comparisons of the signals presented by endogenous and artificial APCs may uncover the precise cues required for CD4⁺ CTL generation. Similarly, here we utilized the MHC II aAPC platform to study which CD4⁺ T cell cues directly enhance CD8⁺ T cell cytotoxicity and memory formation, in the absence of confounding DC intermediaries. Interestingly, these studies revealed an immunostimulatory effect of IL-10 on CD8⁺ T cell cytotoxicity and effector function, in contrast with many studies that demonstrate IL-10 elicits T cell immunosuppression and anergy. Dennis et al.,2013. Our findings and other reported results, Steinbrink et al., 2002; Groux et al., 1998, indicate that the anti-inflammatory functions of IL-10 occur indirectly through suppression of APC function. whereas the direct effects of IL-10 on CD8⁺ T cells are stimulatory. Naing et al., 2018; Emmerich et al., 2012; Mumm et al., 2011; Saxton et al., 2021. By providing stable presentation of MHC and costimulatory molecules, aAPCs are uniquely poised to exploit the direct effects of IL-10 on enhancing CD8⁺ T cell antitumor function. In addition to the therapeutic implications of these findings, they are demonstrative of how a simplified approach using aAPCs can uncover additional aspects of the T cell help process that are difficult to study using traditional cellular approaches.

### 1.5 Methods

### 1.5.1 Mice

Permission for animal experiments was granted by the Johns Hopkins University's Animal Care and Use Committee under Protocol Number: M020M349. Similar numbers of male and female mice ranging from 8-12 weeks were used for experiments, and mice were maintained in adherence of committee guidelines. C57BL/6 (B6, Strain #: 000664), CD45.1 (Strain #: 002014), SMARTA-1 (Strain #: 030450), and OT-II (Strain #: 004194) mice were purchased from Jackson Laboratories (Bar Harbor, ME, USA). PMEL TCR transgenic mice (Jackson Strain #: 005023) were a gift from Nicholas Restifo (National Institutes of Health, MD, USA), and OT-IxRag2−/− mice (Taconic, Strain #: 2334) were a gift from Jonathan Powell (Johns Hopkins University, MD, USA). 2C TCR transgenic mice, Sha et al., 1998, were maintained as heterozygotes by breeding on a B6 background. Mice were housed in a specific pathogen free animal facility on a 12 light/12 dark light cycle, 65-426 75° F., and 40-60% humidity. Experimental and control animals were co-housed.

### 1.5.2 Human Studies

All uses of human material in this study have been approved by the ethical committee of the Johns Hopkins University, and all recruited volunteers provided written informed consent. Volunteers used in this study included two males, ages 27 and 55, and a female, age 32, each of whom was compensated for their blood donation ($20/80 mL). HLA DR4 typing was performed on donor PBMC using an NFLD.D.1 antibody. Drover et al., 1994.

### 1.5.3 Cells

B16-SIY was a gift from Thomas Gajewski (The University of Chicago, IL, USA), B16- F10 (ATCC no. CRL-6475) was a gift from Charles Drake (Johns Hopkins University, MD. USA), and B16-OVA was a gift from Jonathan Powell (Johns Hopkins University, MD, USA). Lymphoblastoid Cell Lines (LCL) were a gift from the Johns Hopkins Human Immunogenetics Laboratory (Johns Hopkins University, MD, USA). Human Jurkat T cells clone E6-1 (ATCC no. TIB-152) and Human Embryonic Kidney (HEK) 293 F cells (Thermo Invitrogen no. R79007) were a gift from Jamie Spangler (Johns Hopkins University, MD, USA). B16 cell lines were cultured in RPMI 1640 medium (Fisher Scientific) containing 10% FBS (Atlanta Biologicals) and 10 M ciproflaxin (Serologicals). B16-OVA and B16-SIY additionally received 400 g/mL geneticin (Gibco). LCLs were cultured in RPMI 1640 medium containing 20% FBS, 200 mM L- glutamine (Gibco), 2 mM HEPES (Quality Biologicals), and 1×Pen/Strep (Gibco). Jurkat T cells were grown in RPMI 1640 media with 10% FBS and 100 U/ml penicillin451 streptomycin (Sigma). Primary murine T cells were cultured in T cell media consisting of RPMI 1640 supplemented with L-glutamine, 1× non-essential amino acids (Gibco), 1 mM sodium pyruvate (Gibco), 0.4× MEM vitamin solution (Gibco), 92 M 2-mercaptoethanol (Gibco), 10 μM ciprofloxacin, and 10% FBS—supplemented with a previously described T cell growth factor cocktail18, unless otherwise indicated. Primary human T cells were cultured in the described T cell culture media containing 10% AB serum (Gemini Bio) instead of FBS and supplemented with additional indicated cytokines. All cells and cell lines were maintained at 37° C. in a humidified atmosphere with 5% CO₂. 1.5.4 Reagents

Recombinant murine IL-2, IL-12p70, IFNy, CCL3, CCL4, CCL5, IL-10, and TNFα and human IL-1P, IL-2, IL-4, IL-6, IL-7, IL-12, IL-15, and IFN-γ were purchased from Peprotech (Cranbury, NJ, USA). Recombinant human IL-2 used in adoptive cell transfer studies (Proleukin) was a gift from Prometheus Laboratories. I-A^{b} OVA₃₂₃₋₃₃₉ (AAHAEINEA), I-A^{b} CLIP₈₇₋₁₀₁ (PVSKMRMATPLLMQA), and I-A^{b} LCMV GP66-77 (DIYKGVYQFKSV) monomers and tetramers were provided by the NIH Tetramer Core Facility (Emory University, GA, USA). DR1 Plasmid was a gift from Luc Teyton (Scripps Research, CA, USA). Soluble DR1 and DR4 monomers were produced in-house, as described below. Day et al., 2003. Soluble Class I MHC-Ig dimers were purified, biotinylated, and loaded with peptides according to previously described approaches. Oelke et al., 2003. The murine/human chimera HA1.7 T cell receptor was produced in-house, as described below. The HLA DR4-restricted NFLD.D.1 hybridoma supernatant was a gift from Sheila Drover (Memorial University of Newfoundland, St. John's, Canada). Drover et al., 1994. A list of all antibodies and their usage is summarized in Table 1. Unlabeled murine and human monoclonal antibodies (anti-CD3 clones 145-2C11 and OKT-3, anti-CD28 clones 37.51 and 9.3, anti-OX40 clone OX-86, anti-IFNγR clone GR-20, anti-I-A/1478 E clone M5/114, anti-TNFα clone XT3.11, and anti-IL-10 clone JES5-2A5) were purchased from BioXCell (West Lebanon, NH, USA). Fluorescently labeled monoclonal antibodies were purchased from BioLegend (San Diego, CA, USA), BD Biosciences (Franklin Lakes, NJ, USA), or eBioscience (San Diego, CA, USA), as indicated below, and used at a 1:100 dilution. OVA₃₂₃₋₃₃₉ peptide was purchased from the Synthesis and Sequencing Facility (Johns Hopkins University, MD, USA). OVA₂₅₇₋₂₆₄ (SIINFEKL), Trp2₁₈₀₋₁₈₈ (SVYVFFDWL), SIY (SIYRYYGL), gp100₂₅₋₃₃ (KVPRNQDWL), HA₃₀₆₋₃₁₈ (PKYVKQNTLKLAT), and NY-ESO-1₁₅₇₋₁₇₀ (SLLMWITQCFLPVF) peptides were purchased from Genscript (Piscataway, NJ, USA).

TABLE 1
Antibody List

| Antibody | Amount | | Clone | Source | Cat. # | Validation |
|---|---|---|---|---|---|---|
| anti-mouse | 100 | ug/mg beads | 145-2C1 | BioXcell | BE0001-1 | T cell |
| CD3e | | | | | | activation |
| anti-mouse | 1 | ug/mL | 37.51 | BioXcell | BE0015-1 | T cell |
| CD28 | | | | | | activation |
| anti-mouse | 1 | ug/mL | OX-86 | BioXcell | BE0031 | T cell |
| OX40 | | | | | | activation |
| anti-mouse | 10 | ug/mL | GR-20 | BioXcell | BE0029 | IFNγR |
| IFNγR | | | | | | neutralization |
| anti-mouse | 10 | ug/mL | M5/114 | BioXcell | BE0108 | MHC II |
| I-A/I-E | | | | | | blockade |
| anti-mouse | 1 | ug/mL | XT3.11 | BioXcell | BE0058 | TNFα |
| TNFα | | | | | | neutralization |
| anti-mouse | 1 | ug/mL | JES5-2A5 | BioXcell | BE0049 | IL-10 |
| IL-10 | | | | | | neutralization |
| anti-human | 100 | ug/mg beads | OKT-3 | BioXcell | BE0001-2 | T cell |
| CD3 | | | | | | activation |
| anti-human | 1 | ug/mL | 9.3 | BioXcell | BE0248 | T cell |
| CD28 | | | | | | activation |

| | | | | | |
|---|---|---|---|---|---|
| PE anti-mouse | 1:100 | 17A2 | BioLegend | 100206 | Flow |
| CD3 | | | | | Cytometry |
| APC anti- | 1:100 | GK1.5 | BioLegend | 100412 | Flow |
| mouse CD4 | | | | | Cytometry |
| PerCP anti- | 1:100 | RM4-5 | BioLegend | 100538 | Flow |
| mouse CD4 | | | | | Cytometry |
| PE anti-mouse | 1:100 | H129.19 | BioLegend | 130310 | Flow |
| CD4 | | | | | Cytometry |
| APC- | 1:100 | GK1.5 | BioLegend | 100414 | Flow |
| Cyanine7 anti- | | | | | Cytometry |
| mouse CD4 | | | | | |
| Alexa Fluor | 1:100 | GK1.5 | BioLegend | 100446 | Flow |
| 594 anti- | | | | | Cytometry |
| mouse CD4 | | | | | |
| APC anti- | 1:100 | 53-6.7 | BioLegend | 100712 | Flow |
| mouse CD8a | | | | | Cytometry |
| PerCP anti- | 1:100 | 53-6.7 | Biolegend | 100732 | Flow |
| mouse CD8 | | | | | Cytometry |
| APC/Cyanine | 1:100 | 53-6.7 | BioLegend | 100714 | Flow |
| 7 anti-mouse | | | | | Cytometry |
| CD8a | | | | | |
| PE/Cyanine7 | 1:100 | 53-6.7 | BD | 561097 | Flow |
| anti-mouse | | | Biosciences | | Cytometry |
| CD8 | | | | | |
| PerCP-Cy5.5 | 1:100 | IM7 | BioLegend | 103032 | Flow |
| anti-mouse | | | | | Cytometry |
| CD44 | | | | | |
| PE anti-mouse | 1:100 | A20 | Biolegend | 110708 | Flow |
| CD45.1 | | | | | Cytometry |
| PE anti-mouse | 1:100 | 104 | BioLegend | 109808 | Flow |
| CD45.2 | | | | | Cytometry |
| APC anti- | 1:100 | MEL-14 | BioLegend | 104412 | Flow |
| mouse CD62L | | | | | Cytometry |
| Alexa Fluor | 1:100 | A7R34 | BioLegend | 135018 | Flow |
| 488 anti- | | | | | Cytometry |
| mouse CD127 | | | | | |
| PE/Cyanine7 | 1:100 | 4B12 | BioLegend | 120124 | Flow |
| anti-mouse | | | | | Cytometry |
| CD197 | | | | | |
| (CCR7) | | | | | |
| Brilliant | 1:100 | 2F1/KLRG1 | BioLegend | 138419 | Flow |
| Violet 605 | | | | | Cytometry |
| anti- | | | | | |
| mouse/human | | | | | |
| KLRG1 | | | | | |
| FITC anti- | 1:100 | M5/114.15.2 | BioLegend | 107606 | Flow |
| mouse I-A/I-E | | | | | Cytometry |
| Alexa Fluor | 1:100 | M5/114.15.2 | BioLegend | 107618 | Flow |
| 647 anti- | | | | | Cytometry |
| mouse I-A/I-E | | | | | |
| PE/Cyanine7 | 1:100 | M5/114.15.2 | BioLegend | 107630 | Flow |
| anti-mouse I- | | | | | Cytometry |
| A/I-E | | | | | |
| FITC anti- | 1:100 | H57-597 | BioLegend | 109206 | Flow |
| mouse TCR β | | | | | Cytometry |
| chain | | | | | |
| APC anti- | 1:100 | H57-597 | BioLegend | 109212 | Flow |
| mouse TCR β | | | | | Cytometry |
| chain | | | | | |
| Alexa Fluor | 1:100 | H57-597 | BioLegend | 109218 | Flow |
| 647 anti- | | | | | Cytometry |
| mouse TCR | | | | | |
| chain | | | | | |
| FITC anti- | 1:100 | FJK-16s | eBioscience | 14-5773-82 | Flow |
| mouse Foxp3 | | | | | Cytometry |
| PerCP- | 1:100 | eBio4B10 | eBioscience | 45-5825-82 | Flow |
| Cyanine5.5 | | | | | Cytometry |
| anti- | | | | | |
| mouse/human | | | | | |
| T-bet | | | | | |
| APC anti- | 1:100 | AFKJS-9 | eBioscience | 17-6988-82 | Flow |
| mouse/human | | | | | Cytometry |
| RORγT | | | | | |
| PE/Cyanine7 | 1:100 | TWAJ | eBioscience | 25-9966-42 | Flow |
| anti- | | | | | Cytometry |
| mouse/human | | | | | |
| Gata3 | | | | | |
| APC anti- | 1:100 | XMG1.2 | BioLegend | 505810 | Flow |
| mouse IFN-γ | | | | | Cytometry |
| PE/Cyanine7 | 1:100 | MP6-XT22 | BioLegend | 506324 | Flow |
| anti-mouse | | | | | Cytometry |
| TNF-α | | | | | |
| PE anti-mouse | 1:100 | JES6-5H4 | BioLegend | 503808 | Flow |
| IL-2 | | | | | Cytometry |
| FITC anti- | 1:100 | GB11 | BioLegend | 515403 | Flow |
| mouse/human | | | | | Cytometry |
| Granzyme B | | | | | |
| Pacific Blue | 1:100 | GB11 | BioLegend | 515408 | Flow |
| anti- | | | | | Cytometry |
| mouse/human | | | | | |
| Granzyme B | | | | | |
| APC anti- | 1:100 | OKT4 | BioLegend | 317416 | Flow |
| human CD4 | | | | | Cytometry |
| PE/Cyanine 7 | 1:100 | A161A1 | BioLegend | 357410 | Flow |
| antihuman | | | | | Cytometry |
| CD4 | | | | | |
| FITC anti- | 1:100 | HI100 | BioLegend | 983002 | Flow |
| human | | | | | Cytometry |
| CD45RA | | | | | |
| APC/Cyanine7 | 1:100 | DREG-56 | BioLegend | 304814 | Flow |
| antihuman | | | | | Cytometry |
| CD62L | | | | | |
| FITC anti- | 1:100 | FN50 | BioLegend | 310904 | Flow |
| human CD69 | | | | | Cytometry |
| PerCP- | 1:100 | FN50 | BioLegend | 310926 | Flow |
| Cyanine5.5 | | | | | Cytometry |
| antihuman | | | | | |
| CD69 | | | | | |
| APC anti- | 1:100 | Ber-ACT8 | BioLegend | 350216 | Flow |
| human CD103 | | | | | Cytometry |
| Brilliant | 1:100 | TU27 | BioLegend | 339010 | Flow |
| Violet 421 | | | | | Cytometry |
| antihuman | | | | | |
| CD122 | | | | | |
| FITC anti- | 1:100 | L243 | BioLegend | 307632 | Flow |
| human HLA | | | | | Cytometry |
| DR | | | | | |
| FITC anti- | 1:100 | 4S.B3 | BioLegend | 502506 | Flow |
| human IFN-γ | | | | | Cytometry |
| PerCP-Cy5.5 | 1:100 | MQ1-17H12 | BioLegend | 500322 | Flow |
| antihuman IL-2 | | | | | Cytometry |
| PE/Cyanine7 | 1:100 | MAb11 | BioLegend | 502930 | Flow |
| antihuman | | | | | Cytometry |
| TNF-α | | | | | |
| FITC anti- | 1:100 | R26-46 | BD | 553434 | Flow |
| mouse Ig λ1 | | | Biosciences | | Cytometry |
| λ2 λ3 light | | | | | |
| chain | | | | | |
| FITC anti- | 1:100 | R19-15 | BD | 553390 | Flow |
| mouse IgG2a | | | Biosciences | | Cytometry |
| FITC anti- | 1:100 | G94-56 | BD | 554008 | Flow |
| hamster IgG | | | Biosciences | | Cytometry |
| FITC anti- | 1:100 | G192-1 | BD | 554026 | Flow |
| hamster IgG | | | Biosciences | | Cytometry |

### 1.5.5 Expression of Human HLA DR Monomers

HLA DR1 and DR4 monomers (Table 2) were produced following a previously described approach. Day et al., 2003. Briefly, synthetic gene fragments (Twist Bioscience) for HLA-DR1 and DR4 a and 3 chains were separately cloned into the gWiz mammalian expression vector (Genlantis) using Gibson Assembly (New England Biolabs). The shared DRα chain vector consisted of the DRα gene (DRA*01:01) linked to a Fos leucine zipper dimerization domain that was further linked to a C-terminal hexahistidine tag. The distinct β chain vectors consisted of the Class II-associated invariant chain peptide (CLIP) followed by a thrombin cleavage site which was linked to the appropriate DRβ gene (DRB1*01:01 for HLA-DR1 or DRβ 1*04:01 for DR4). The DRβ gene was further linked to a Jun leucine zipper dimerization domain and C-terminal hexahistidine tag. Plasmids were purified using ZymoPURE II Plasmid Midiprep Kit (Zymo Research). All constructs were verified by Sanger sequencing. HLA-DR1 and DR4 MHC proteins were expressed in a HEK 293-F mammalian cell expression system. HEK 293-F cells were cultivated in Freestyle 293 Expression Medium (Thermo Invitrogen), supplemented with 10 U/mL penicillin-streptomycin (Gibco). All cell lines were maintained at 37° C. in a humidified atmosphere with 5% CO₂. HEK 293F cells were maintained on a shaker set to 125 rpm. HLA-DR1 and DR4 monomers were expressed recombinantly in human embryonic kidney (HEK) 293-F cells via transient co-transfection of plasmids encoding the respective DRα and DRβ chains. DRα and DRβ chain plasmids were titrated in small scale co-transfection tests to determine optimal DNA ratios for large-scale expression. HEK 293F cells were grown to 1.2×10⁶ cells/mL and diluted to 1.0×10⁶ cells/mL on the day of transfection. Plasmid DNA (filter sterilized though a 0.22- m PES filter Corning]) and polyethyleneimine (PEI, Polysciences) were independently diluted to 0.05 and 0.1 mg/mL, respectively, in OptiPro medium (Thermo Invitrogen), and incubated at 20° C. for 15 min. Equal volumes of diluted DNA and PEI were mixed and incubated at 20° C. for an additional 15 min. Subsequently, the DNA/PEI mixture (40 mL per Liter cells) was added to a flask containing the diluted HEK cells. which was then incubated at 37° C. with shaking for 3-5 days. Secreted protein was harvested from HEK 293F cell supernatants by via Ni-NTA (Expedeon) affinity chromatography, followed by size exclusion chromatography on an AKTA fast protein liquid chromatography (FPLC) instrument using a Superdex 200 column (Cytiva). All proteins were stored in HEPES buffered saline (HBS, 150-mM NaCl in 10 mM HEPES pH 7.3). Purity was verified by SDS-PAGE analysis.

TABLE 2
HLA DR monomer and HA1.7 construct sequences

| | |
|---|---|
| DRα | MAISGVPVLGFFIIAVLMSAQESWAIKEEH |
| | VIIQAEFYLNPDQSGEFMFDFDGDEIFHVD |
| | MAKKETVWRLEEFGRFASFEAQGALANIAV |
| | DKANLEIMTKRSNYTPITNVPPEVTVLTNS |
| | PVELREPNVLICFIDKFTPPVVNVTWLRNG |
| | KPVTTGVSETVFLPREDHLFRKFHYLPFLP |
| | STEDVYDCRVEHWGLDEPLLKHWEFDAPSP |
| | LPETTEVDGGGGGLTDTLQAETDQLEDEKS |
| | ALQTEIANLLKEKEKLEFILAAGGSGGSGL |
| | NDIFEAQKIEWHEH HHHHH |
| | (SEQ ID NO: 1) |
| DR1β | MAISGVPVLGFFIIAVLMSAQESWAIKEEH |
| | VIIQAEFYLNPDQSGEFMFDFDGDEIFHVD |
| | MAKKETVWRLEEFGRFASFEAQGALANIAV |
| | DKANLEIMTKRSNYTPITNVPPEVTVLTNS |
| | PVELREPNVLICFIDKFTPPVVNVTWLRNG |
| | KPVTTGVSETVFLPREDHLFRKFHYLPFLP |
| | STEDVYDCRVEHWGLDEPLLKHWEFDAPSP |
| | LPETTEVDGGGGGLTDTLQAETDQLEDEKS |
| | ALQTEIANLLKEKEKLEFILAAGGSGGSGL |
| | NDIFEAQKIEWHEH HHHHH |
| | (SEQ ID NO: 2) |
| DR4β | MVCLKLPGGSCMTALTVTLMVLSSPLALAG |
| | DTGLPVSKMRMATPLLMQASGGGSLVPRGS |
| | GGGGSGDTRPRFLWQLKFECHFFNGTERVR |
| | LLERCIYNQEESVRFDSDVGEYRAVTELGR |
| | PDAEYWNSQKDLLEQRRAAVDTYCRHNYGV |
| | GESFTVQRRVEPKVTVYPSKTQPLQHHNLL |
| | VCSVSGFYPGSIEVRWFRNGQEEKAGVVST |
| | GLIQNGDWTFQTLVMLETVPRSGEVYTCQV |
| | EHPSVTSPLTVEWRARSESAQSKVDGGGGG |
| | RIARLEEKVKTLKAQNSELASTANMLREQV |
| | AQLKQKVMNH (SEQ ID NO: 3) |
| HA1.7 | MLLLLVPVLEVIFTLGGTRAQSVTQLGSHV |
| | SVSEGALVLLRCNYSSSVPPYLFWYVQYPN |
| | QGLQLLLKYTSAATLVKGINGFEAEFKKSE |
| | TSFHLTKPSAHMSDAAEYFCAVSESPFGNE |
| | KLTFGTGTRLTIIPIQNPEPAVYQLKDPRS |
| | QDSTLCLFTDFDSQINVPKTMESGTFITD |
| | KTVLDMKAMDSKSNGAIAWSNQTSFTCQDI |
| | FKETNATYPSSDVPCDATLTEKSFETDMNL |
| | NFQNLSVMGLRILLLKVAGFNLLMTLRLWS |
| | SRRKRGSGATNFSLLKQAGDVEENPGPMGI |
| | RLLCRVAFCFLAVGLVDVKVTQSSRYLVK |
| | RTGEKVFLECVQDMDHENMFWYRQDPGLGL |
| | RLIYFSYDVKMKEKGDIPEGYSVSREKKER |
| | FSLILESASTNQTSMYLCASSSTGLPYGYT |
| | FGSGTRLTVVEDLRNVTPPKVSLFEPSKAE |
| | IANKQKATLVCLARGFFPDHVELSWWVNGK |
| | EVHSGVSTDPQAYKESNYSYCLSSRLRVSA |
| | TFWHNPRNHFRCQVQFHGLSEEDKWPEGSP |
| | KPVTQNISAEAWGRADCGITSASYHQGVLS |
| | ATILYEILLGKATLYAVLVSGLVLMAMVKK |
| | KNS |
| | (SEQ ID NO: 4) |

### 1.5.6 Biotinylation, Thrombin Cleavage, Peptide Exchange, and Tetramerization of Human HLA DR Monomers

For preparation of biotinylated HLA-DR1 and DR4, a C-terminal biotin acceptor peptide (BAP) GLNDIFEAQKIEWHE sequence was added to the previously described HLA-DR expression vectors. Following transfection and Ni-NTA affinity chromatography, the HLA-DR monomers were biotinylated with the soluble BirA ligase enzyme in 0.5 mM Bicine pH 8.3, 100 mM ATP, 100 mM magnesium acetate, and 500 mM biotin (Sigma). After overnight incubation at 4° C., excess biotin was removed by size-exclusion chromatography on an AKTA FPLC instrument using a Superdex 200 column (Cytiva). To confirm covalent attachment of biotin, at least I g of each biotinylated HLA-DR protein was incubated with 2 mL of streptavidin (5 mg/mL, MilliporeSigma) at 20° C. for 5 min followed by SDS-PAGE analysis to confirm a shift in molecular weight. CLIP peptides were cleaved by incubating DR proteins with 20 U of thrombin (Novagen, Madison WI) per milligram of monomer at 37° C. for 2 hours. Peptide exchange was then performed by adjusting the concentration of monomer to 3.3 μM in a peptide exchange buffer consisting of 50 mM sodium citrate pH 5.2, 1% octylglucoside (ThermoFisher), 100 mM NaCl and 1× protease inhibitor cocktail (Roche) and incubating with 50 M of peptide overnight at 37° C. To remove excess peptide, monomers were then washed three times in PBS with a 10 kDA MWCO concentrator (Sigma) and then frozen in small aliquots at −80° C. Multimerization reactions were performed through incremental addition of fluorescent streptavidin molecules (Agilent) to biotinylated monomer at 20° C. to reach a final streptavidin to monomer ratio of 1:3.5.

1.5.7 Synthesis of aAPCs

Murine I-A^{b} CLIP and I-A^{b} OVA and murine and human αCD3/αCD28 microparticles (Dynal, Lake Success, New York) were synthesized according to the manufacturer's instructions and as previously described. Oelke et al., 2003. Murine and human nanoparticle aAPCs were synthesized as previously described, Hickey et al., 2020, and in accordance with the manufacturer's instructions by incubating 200 nm NHS-activated magnetic beads (Ocean Nanotech, Springdale, AR, USA) with either I-A^{b}, DR1, DR4, K^{b}—Ig or D^{b}-Ig monomers, dimers, or fluorescently labelled tetramers. Combined Signal 1 and Signal 2, Signal 1 and Isotype, or Signal 1 and BSA aAPCs were produced by pre-mixing monomers or dimers at a 1:1 or 1:3 molar ratio, as indicated, with mouse or human αCD28, isotype Armenian hamster IgG antibodies Clone HTK888 (Biolegend), or Bovine Serum Albumin (GeminiBio). Combined MHC I and MHC II aAPCs were produced by pre-mixing I-A^{b} monomers with K^{b}-Ig dimers at a 1:1 molar ratio. Human aAPCs underwent thrombin cleavage and peptide exchange post conjugation of DR-CLIP proteins. Briefly, aAPCs were incubated with 40 units of thrombin per milligram of conjugated DR protein at 37° C. for 2 hours. Particles were then magnetically washed and resuspended at 30 nM conjugated protein in peptide exchange buffer and then incubated overnight at 37° C. with 3 M peptide. Finally, particles were washed and resuspended either in storage buffer (1× PBS and 0.05% BSA) or human T cell culture media.

1.5.8 Characterization of aAPCs

Nanoparticle were sized using a Zetasiser DLS and imaged using Transmission Electron Microscopy (TEM). For TEM, iron dextran nanoparticles were allowed to adhere on carbon coated copper support grids (EMS CF400-Cu-UL) for 2 minutes, rinsed three times with deionized water, and allowed to dry at 20° C. The grids were mounted and imaged on a transmission electron microscope (Hitachi 7600) at an acceleration voltage of 80 kV. Protein conjugation to Dynal microparticles was characterized by staining microparticles with FITC labelled secondary antibodies and then comparing them to a standard curve based on a Quantum FITC-5 MESF fluorescence quantification kit (Bangs Laboratories). Protein conjugation to nanoparticle aAPCs was performed as previously described, Komides et al., 2017, by staining particles with FITC labelled secondary antibodies, magnetically washing the particles, and then comparing their absorbance at 405 nm (Beckman Coulter AD340) and fluorescence at 485 nm (FisherScientific Varioskan LUX) to standard curves of known bead and protein concentrations, respectively. The following secondary antibodies were used: FITC anti-hamster IgG clone G94-56 (BD Biosciences) for murine αCD3, FITC anti-hamster IgG clone G192-1 (BD Biosciences) for murine αCD28, FITC anti-mouse I-A/I-E clone M5/114.15.2 (BioLegend) for murine IAb, FITC anti-mouse Ig λ1 λ2 λ3 light chain clone R26-46 (BD Biosciences) for murine K^{b}-Ig and D^{b}-Ig, FITC anti-mouse IgG2a clone R19-15 (BD Biosciences) for human αCD3 and αCD28, and FITC anti-human HLA DR clone L243 (BioLegend) for human DR1 and DR4. For fluorescent tetramer-labelled nanoparticles, the protein concentration per nanoparticle was determined by comparing the fluorescence of the particles to a standard curve of unconjugated fluorescent tetramer.

1.5.9 T cell isolation

OT-II, SMART-A1, or B6 mice were used for CD4⁺ expansions, and OT-I, 2C, PMEL, and B6 mice were used for CD8⁺ expansions. Spleens and lymph nodes were harvested from 8 to 12-week-old mice and processed through a 70- m cell strainer. Then, CD4⁺ and CD8⁺ T cells were isolated using corresponding no-touch isolation kits and magnetic columns from Miltenyi Biotech (Auburn, CA, USA) according to the manufacturer's instructions.

For human isolations, blood was drawn from healthy donors per JHU IRB approved protocols and PBMC were isolated by Ficoll-Paque PLUS (GE Healthcare) density gradient centrifugation. Cells were cryopreserved in a 90% FBS, 10% DMSO solution at 10⁷ cells/mL and stored in liquid nitrogen. Prior to use, cryopreserved PBMC were thawed with 50 U/mL benzonase Nuclease HC (EMD Millipore), washed, and then incubated overnight in T cell culture medium at 37° C. The following morning, CD4⁺ T cells were purified using no-touch CD4⁺ isolation kits and magnetic columns (Miltenyi).

### 1.5.10 Bone Marrow Derived Dendritic Cell Isolation

Bone marrow derived dendritic cells (BMDC) were generated following a well-established approach. Lutz et al., 1999. Marrow was flushed from femurs and tibia of B6 mice, filtered, red blood cells lysed, washed, and cultured in non-treated 6 well plates at 1×10⁶ cells/mL in DC media containing RPMI 1640 media (Gibco) supplemented with 10% FBS, 1% Pen/Strep (Gibco), 50 [M 2-mercaptoethanol (Gibco), and 20 ng/mL GM-CSF (Peprotech). On day 3, cells were refed with DC media containing 40 ng/mL GM-CSF. On day 6, 50% of cell supernatant was replaced with DC media containing 20 ng/mL GM-CSF. On day 8, non-adherent or loosely adherent cells were harvested and matured overnight by replating cells at 1×10⁶ cells/mL in DC media containing 100 ng/mL lipopolysaccharide (Sigma Aldrich), 20 ng/mL GM-CSF, and 1 μM of peptide. Prior to stimulation of CD4⁺ T cells, DC maturation was confirmed via flow cytometry by staining for FITC anti-mouse CD11b clone M1/70 (BD Biosciences), PerCP-Cy5.5 anti- mouse CD11c clone N418 (BioLegend), APC anti-mouse CD86 clone GL-1 (BioLegend), Live/Dead Fixable Violet (Invitrogen), BV605 anti-mouse F4/80 clone BM8 (BioLegend), PE anti-mouse CD80 clone 16-10A1 (BioLegend), and PE-Cy7 anti-mouse I-A/I-E clone M5/114.15.2 (BioLegend). 1.5.11 Ex vivo T cell expansion Isolated murine CD4⁺ T cells were cultured in T cell culture media with the addition of either a previously described optimized T cell growth factor cocktail (TF), Oelke et al., 2000, IL-2 (10 ng/mL), or various combinations of a Th1 skewing media composed of IL-2, IL-12p70, and IFN-γ (each at 10 ng/mL). Cells were plated on day 0 at 10⁵ cells/mL and refed on day 3 of culture, with half of the initial volume of T cell culture media and twice the concentration of cytokines. On day 0, micro-aAPCs were added at a 1:1 particle to cell ratio, whereas nano-aAPCs were added at a concentration of 80 ng/mL of conjugated I Ab, unless otherwise indicated. For aAPCs lacking Signal 2 on their surface, soluble UCD28 was added at a concentration of 1 μg/mL unless otherwise indicated. For peptide-based stimulations, isolated splenocytes were plated at 8×10⁵ cells/mL in T cell culture media with the addition of 1 g/mL of peptide. For BMDC-based stimulations, murine CD4⁺ T cells were plated at 10⁵ cells/mL and at a 1:1 ratio with mature BMDCs in T cell culture media.

Murine CD4⁺ T cell proliferation was assessed by labelling a subset of isolated CD4⁺ T cells on day 0 with carboxyfluorescein succinimidyl ester (CFSE, Invitrogen). Cells were incubated with 5 M dye in T cell culture media at 37° C. for 20 minutes, washed and plated as above, and on day 3 of culture harvested and assessed for CFSE dilutions on a BD FACSCalibur flow cytometer. Another subset of unlabeled cells was plated as above and, on day 7, harvested, stained with Trypan blue to exclude dead cells, and then manually counted with a hemocytometer. Fold expansion was calculated as the ratio of live cells at days 7 and 0. Cell phenotype and function was assessed, as described below.

Isolated murine CD8⁺ T cells were cultured as above in T cell culture media supplemented with TF. Class I aAPCs were added at a concentration of 30 ng/mL of conjugated K^{b} or D^{b} and 1 μg/mL soluble αCD28 unless otherwise indicated. Cells were refed as above on day 3 and then harvested and counted on day 7 for functional and phenotypic analyses. For some experiments, T cell culture media was additionally supplemented at day 0 with 25 ng/mL IL-10 or 5 ng/mL TNF-α, and then refed with double these concentrations and half the initial volume on day 3. For murine CD4⁺ and CD8⁺ co-culture experiments, CD8⁺ T cells were mixed at a 1:1 ratio with either freshly isolated CD4⁺ T cells or CD4⁺ T cells activated with S1+S2 aAPCs (80 ng/mL conjugated I-A^{b} and 1 g/mL αCD28) for 5 days in Th1 media. The CD4:CD8 mixture was then plated at 10⁵ cells/mL in T cell culture media supplemented with TF, MHC I aAPCs (30 ng/mL), MHC II aAPCs (80 ng/mL), and soluble αCD28 (1 g/mL), unless otherwise indicated. For some experiments, T cell culture media was additionally supplemented at days 0 and 3 with 1 μg/mL IL-10 or TNF-α blocking antibodies. Cells were refed as above on day 3 and harvested and counted on day 7 for further functional and phenotypic analyses. The relative ratios of CD4⁺ and CD8⁺ T cells over the co-culture period was tracked via flow cytometry by staining cells with APC anti-mouse CD4 clone GK1.5 (Biolegend), PE anti-mouse CD3 clone 17A2 (Biolegend), FITC anti-mouse CD8a clone 53-6.7 (BD Biosciences), and Live/Dead Fixable Violet (Invitrogen). CD4⁺ and CD8⁺ co-culture experiments were also performed in 0.4-μm pore-size polycarbonate membranes transwell plates (Costar). 10⁵ OT-I CD8⁺ T cells were placed in the lower compartment in 0.75 mL of T cell culture media supplemented with T cell growth factor, MHC I aAPCs (30 ng/mL conjugated K^{b}) and 1 g/mL αCD28. 10⁵ Day 5 Th1 OT-II CD4⁺ T cells were either separated in the upper or mixed with the CD8⁺ T cells in the lower compartment in an additional 0.75 mL of T cell culture media supplemented with TF, MHC II aAPCs (80 ng/mL conjugated I-A^{b}), and 1 g/mL αCD28. Cells were refed as above on day 3 and harvested and counted on day 7 for further functional and phenotypic analyses.

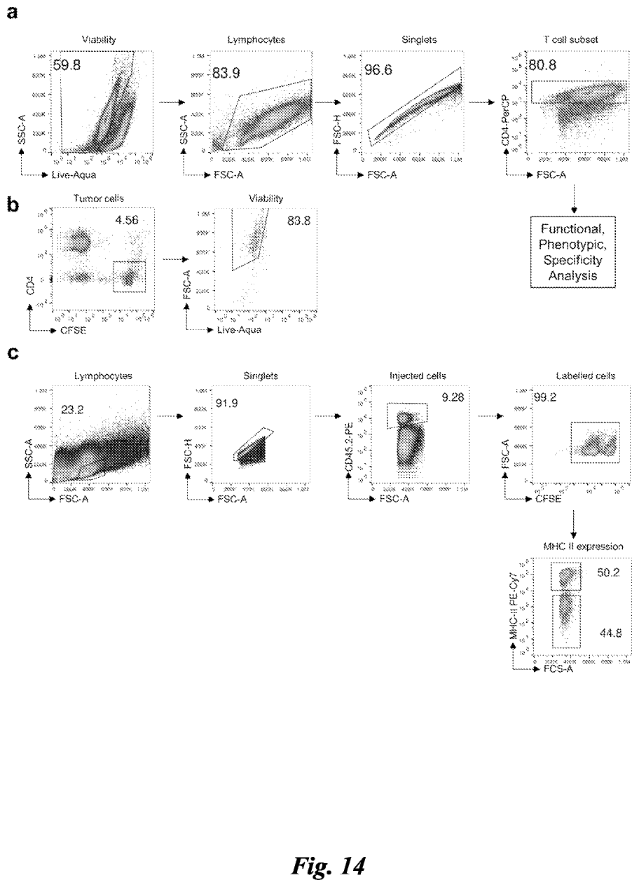

For human T cell expansions, the day 0 precursor frequencies of HA₃₀₆_₃₁₈ CD4⁺ T cells was assessed through tetramer staining. Isolated CD4⁺ T cells were then seeded at 10⁶ cells/mL in human T cell culture medium with indicated cytokines, and peptide exchanged Class II aAPCs were added at a concentration of 30 ng/mL of conjugated DR4. On days 3, 5, 10, 12, 17, and 19, cells were refed with one quarter of the initial volume of T cell culture media and twice the concentration of cytokines, and on days 7, 14, and 21, cells were harvested, counted, and assessed for antigen specificity, phenotype, and function. On days 7 and 14 cells were additionally re-plated with fresh media, cytokines, and aAPCs at 5×10⁵ cells/mL and 100 ng/mL DR4 (day 7) and 3×10⁵ cells/mL and 100 ng/mL DR4 (day 14). respectively. Fold proliferation at days 7, 14, and 21 was calculated as the ratio of live tetramer positive CD4⁺ T cells (total number of cells multiplied by the percentage of live lymphocytes that were both CD4 and tetramer positive) at the current and previous time points. Representative gating strategies for ex vivo T cell expansion studies can be found in FIG. 14a.

### 1.5.12 Ex Vivo T Cell Phenotypic Studies

Lineage specific transcription factors of naive or expanded murine CD4⁺ T cells were analyzed by washing cells and staining them for 15 minutes at 4° C. with Live/Dead Fixable Aqua (Invitrogen) and APC-Cyanine7 anti-mouse CD4 clone GK1.5 (BioLegend). Cells were then washed, fixed, and permeabilized using the Foxp3 Transcription Factor Staining Buffer Set (eBioscience), and then stained for FITC anti- mouse Foxp3 clone FJK-16s (eBioscience), PerCp-Cyanine5.5 anti-mouse/human T bet clone eBio4B10 (eBioscience), APC anti-mouse/human RORγT clone AFKJS-9 (eBioscience), and PE/Cyanine7 anti-mouse/human Gata3 clone TWAJ (eBioscience), or their corresponding isotypes. Finally, cells were washed and resuspended in FACS wash buffer (1× PBS, 2% FBS, 0.5% sodium azide) and then analyzed on an Attune N×T Flow Cytometer.

The memory phenotype of naive or expanded murine CD4⁺ or CD8⁺ T cells was analyzed by harvesting cells, and then washing and staining them for 15 minutes at 4° C. with Live/Dead Fixable Violet (Invitrogen), PE anti-mouse CD3 clone 17A2 (BioLegend), APC/Cyanine 7 anti-mouse CD4 clone GK1.5 (BioLegend) or APC/Cyanine 7 anti- mouse CD8a clone 53-6.7 (BioLegend), Alexa Fluor 488 anti-mouse CD127 clone A7R34 (BioLegend), PerCP-Cy5.5 anti-mouse CD44 clone IM7 (BioLegend), APC anti- mouse CD62L clone MEL-14 (BioLegend), Brilliant Violet 605 anti-mouse/human KLRG1 clone 2F1/KLRG1 (BioLegend), and PE/Cyanine7 anti-mouse CD197 (CCR7) clone 4B12 (BioLegend), or their corresponding isotypes. For rare T cell analysis, PE-labelled multimer staining was substituted for anti-CD3 (see below) and performed prior to other surface marker staining. The memory phenotype of human CD4⁺ T cells was analyzed by first staining cells with PE labelled tetramers (see below), and then staining them for 15 minutes at 4° C. with Live/Dead Fixable Aqua, PE/Cyanine 7 anti-human CD4 clone A161 A1 (BioLegend), FITC anti-human CD45RA clone HI100 (BioLegend), APC/Cyanine7 anti-human CD62L clone DREG-56 (BioLegend), PerCP-Cyanine5.5 anti-human CD69 clone FN50 (BioLegend), APC anti-human CD103 clone Ber-ACT8 (BioLegend), and Brilliant Violet 421 anti-human CD122 clone TU27 (BioLegend), or their corresponding isotypes. Representative gating strategies for ex vivo T cell phenotypic studies can be found in FIG. 14a.

### 1.5.13 Ex Vivo T Cell Functional Studies

Intracellular cytokine staining of murine CD4⁺ and CD8′ T cells was performed by diluting them to approximately 2×10⁶ cells/mL in T cell culture media and incubating them at 37° C. for 6 hours with 1× cytokine activation cocktail (BioLegend) and GolgiPlug (BD Biosciences). No stimulation controls received only GolgiPlug. Following incubation, cells were washed and stained with PerCP anti-mouse CD4 clone RM4-5 (BioLegend) or PerCP anti-mouse CD8 clone 53-6.7 (Biolegend) and Live/Dead Fixable Aqua (Invitrogen) for 15 minutes at 4° C. Cells were then fixed and permeabilized overnight with the Cytofix/Cytoperm Fixation/Permeabilization kit (BD Biosciences), washed, and stained with APC anti-mouse IFN-γ clone XMG1.2 (BioLegend), PE/Cyanine7 anti-mouse TNF-α clone MP6-XT22 (BioLegend), PE anti-mouse IL-2 clone JES6-5H4 (BioLegend), and FITC anti-mouse/human Granzyme B clone GB11 (BioLegend). Cells were then washed and resuspended in FACS wash buffer and analyzed on an Attune N×T Flow Cytometer.

For cytokine analysis of antigen-specific murine CD8⁺ T cells, a similar assay was used with the following modifications. Prior to stimulation, T cells were stained with cognate and non-cognate biotinylated pMHC-Ig dimers (see below), washed, and then re-stimulated. After the 6 hour incubation, cells were washed and stained with or PerCP anti-mouse CD8 clone 53-6.7 (BioLegend), PE-labeled streptavidin (BD Biosciences), and Live/Dead Fixable Aqua (Invitrogen) for 15 minutes at 4° C. Cells were then fixed and permeabilized and stained with APC anti-mouse IFN-γ clone XMG1.2 (BioLegend), PE/Cyanine7 anti-mouse TNF-α clone MP6-XT22 (BioLegend), and FITC anti-mouse/human Granzyme B clone GB11 (BioLegend). Cells were then washed and resuspended in FACS wash buffer and analyzed on an Attune N×T Flow Cytometer. Antigen-specific human CD4⁺ T cell cytokine analysis was performed by pulsing LCLs with 10 μg/mL cognate (HA₃₀₆₋₃₁₈) or irrelevant (NY-ESO-1161-180) peptide for 1 hour at 20° C., washing, and then incubating them 1:1 with T cells in human T cell culture media containing GolgiPlug for 5 hours at 37° C. Tetramer staining was begun 50 minutes prior to the end of the 5 hour incubation (see below). Afterwards, cells were washed and stained for APC anti-human CD4 clone OKT4 (BioLegend) and Live/Dead Fixable Aqua (Invitrogen). Cells were then fixed and permeabilized as above and stained for FITC anti-human IFN-γ clone 4S.B3 (BioLegend), PerCP-Cy5.5 anti-human IL-2 clone MQ1- 17H12 (BioLegend), Pacific Blue anti-mouse/human Granzyme B clone GB11 (BioLegend), and PE/Cyanine7 anti-human TNF-α clone MAb 11 (BioLegend). Cells were then washed and resuspended in FACS wash buffer and analyzed on an Attune N×T Flow Cytometer. Representative gating strategies for ex vivo T cell functional studies can be found in FIG. 14a.

In vitro killing assays of murine CD4⁺ and CD8⁺ T cells were performed as previously described, Hickey et al., 2020, by labelling 5×10⁶ B16 tumor cells with 5 M CFSE dye (Invitrogen) at 37° C. for 20 minutes in 1 mL PBS. The reaction was quenched by adding 5 mL FBS and incubating cells at 37° C. for 5 minutes. Tumor cells were plated at 5×10⁴ cells/mL in ultra-low cluster 96 well plates (Costar) co-incubated with T cells at varying effector to target ratios (30:1, 10:1, 1:1, 0.1:1, 0.01:1, and 0:1) at 37° C. for 16 hours. For blocking studies, anti-I-A/I-E clone M5/114 (BioXcell) or anti-IFNγR clone GR-20 (BioXcell) as well as their corresponding isotype controls were added at 10 g/mL, while Granzyme B inhibitor Z-AAD-CMK (Calbiochem) was added at 25 [M. Cells were then treated with trypsin to detach plate-bound tumor cells, stained for 15 minutes at 4° C. with Live/Dead Fixable Aqua (Invitrogen) and APC anti-mouse CD4 clone GK1.5 (BioLegend) or APC anti-mouse CD8a clone 53-6.7 (BioLegend), washed, and then run analyzed on an Attune N×T Flow Cytometer. To monitor MHC II expression on live tumor cells, cells were instead stained with Live/Dead Fixable Violet (Invitrogen), APC anti-mouse CD4 clone GK1.5 (BioLegend), and PE/Cyanine7 anti-mouse I-A/I-E clone M5/114.15.2 (BioLegend). Representative gating strategies for in vitro killing assays can be found in FIG. 14b.

### 1.5.14 Multimer Staining

Murine CD4⁺ T cell tetramer staining was performed by incubating 1×10⁵ cells at 37° C. for 2 hours with 60 μg/mL cognate and non-cognate I-A^{b} tetramers (NIH Tetramer Core Facility) in T cell culture medium. Cells were then washed in PBS, stained with APC anti-mouse CD4 clone GK1.5 (BioLegend) and Live/Dead Fixable Green (Invitrogen) for 15 minutes at 4° C., washed and resuspended in FACS Wash Buffer, and then analyzed on an Attune N×T Flow Cytometer. Murine CD8⁺ T cell dimer staining was performed by incubating 1×10⁵ cells at 4° C. for 1 hour with 10 [g/mL cognate and non-cognate biotinylated K^{b}-Ig or D^{b}-Ig dimers (in-house) in FACS Wash Buffer. Cells were then washed in PBS, stained with APC anti-mouse CD8a clone 53-6.7 (BioLegend) and Live/Dead Fixable Green (Invitrogen) for 15 minutes at 4° C., washed and resuspended in FACS Wash Buffer, and then analyzed on an Attune N×T Flow Cytometer.

Human CD4⁺ T cell tetramer staining was performed by incubating 1×10⁵ cells at 20° C. for 5 minutes with 40 L/mL Human TruStain FcX Fe Receptor Blocking Solution (BioLegend) in T cell culture medium. An additional 20 minute incubation at 37° C. with 50 nM dasatinib (Axon Medchem) followed by a 30 minute incubation at 37° C. with 20 μg/mL cognate and non-cognate tetramers (in-house) was then done. Cells were then washed in PBS, stained with APC anti-human CD4 clone OKT4 (BioLegend) and Life/Dead Fixable Green (Invitrogen) for 15 minutes at 4° C., washed and resuspended in FACS Wash Buffer, and then analyzed on an Attune N×T Flow Cytometer. Representative gating strategies for multimer staining can be found in FIG. 14a.

### 1.5.15 T Cell Binding, Internalization, Enrichment, and Combined Enrichment and Expansion

Murine CD4⁺ T cell binding studies were performed by incubating 1×10⁵ recently isolated OT-II, SMART-A1, or B6 CD4⁺ T cells for 30 minutes at 37° C. in T cell culture media with varying concentrations of nano- and micro-aAPCs. Cells were then washed, stained for 15 minutes at 4° C. in FACS Wash Buffer with FITC anti-mouse I-A/I-E clone M5/114.15.2 (BioLegend) and APC anti-mouse CD4 clone GK1.5 (BioLegend) to detect aAPC-bound CD4⁺ T cells, washed again, and then analyzed on a BD FACSCalibur Flow Cytometer.

Murine CD4⁺ T cell internalization studies were performed as above using nanoparticles coated with PE-labelled I-A_{OVA}^{b} tetramers at 80 ng I-A^{b}/10⁵ CD4⁺ T cells. The incubation time was varied between 30 and 120 minutes, incubation temperature between 4° C. and 37° C., and incubation media between T cell culture with and without 0.5% sodium azide (NaN₃) supplementation. Cells were then washed and stained for 15 minutes at 4° C. in FACS Wash Buffer with FITC anti-mouse TCR P chain clone H57-597 (BioLegend), APC anti-mouse CD4 clone GK1.5 (BioLegend), and PE-Cy7 anti-mouse T-A/T-E clone M5/114.15.2 (BioLegend). Samples were then washed again and analyzed on an Attune N×T flow cytometer for the percentage of cells with surface-bound (Tetramer⁺MHC II⁺) versus internalized (Tetramer⁺MHC II⁻) aAPCs.

OT-II doped enrichment studies were performed by CFSE labelling recently isolated OT-II CD4⁺ T cells with 5 μM CFSE (Invitrogen) in T cell culture medium for 20 minutes at 37° C. and then diluting them 1:1000 with recently isolated, unlabeled B6 CD4⁺ T cells. Cells were then incubated for 2 hours with micro- or nano-aAPCs at 37° C. in T cell culture media and then magnetically enriched using a 96-well ring magnet. Hickey et al., 2020, For some experiments, the incubation was performed at 4° C. or with T cell culture media supplemented with 0.5% sodium azide (NaN₃). The enriched fraction was then counted with a hemocytometer, washed, and stained at 4° C. for 15 minutes with APC anti-mouse CD4 clone GK1.5 (BioLegend) in FACS Wash Buffer. Cells were then washed and analyzed on a BD FACSCalibur Flow Cytometer. Fold enrichment and percent cell recovery were calculated by taking the ratio of both the frequency and number of CFSE⁺ CD4⁺ T cells pre and post enrichment. To track aAPC internalization during the enrichment process, diluted cells were incubated with nano-aAPCs conjugated with PE-labelled tetramers at 30 ng I-Ab/10⁶ CD4⁺ T cells, as above. Both the enriched and unenriched fractions were collected, counted with a hemocytometer, washed, and stained at 4° C. for 15 minutes with PerCP anti-mouse CD4 clone RM4-5 (Biolegend), PE-Cy7 anti-mouse I-A/I-E clone M5/114.15.2 (BioLegend), and Alexa Fluor 647 anti-mouse TCR R chain clone H57-597 (BioLegend). Samples were then washed and analyzed on an Attune N×T Flow Cytometer, monitoring, as above, the percentage of cognate (CFSE⁺) and irrelevant (CFSE⁻) cells with surface-bound (Tetramer⁺MHC II⁺) versus internalized (Tetramer⁺MHC II⁻) aAPCs. SMART-A1 doped enrichment studies were performed analogously, except unlabeled SMART-A1 cells were used instead and detected with a PE anti-mouse CD45.1 clone A20 (Biolegend) antibody.

Doped enrichment and expansion studies were performed by diluting freshly isolated, unlabeled OT-II or SMART-A1 CD4⁺ T cells into recently isolated, unlabeled B6 CD4⁺ T cells. Cells were then incubated for 2 hours with 30 ng conjugated I-A^{b}/10⁶ CD4⁺ T cells of S1 aAPCs at 37° C. in T cell culture media and then magnetically enriched using a 96- well ring magnet. Hickey et al., 2020. The enriched fractions were plated at 2.5×10⁵ cells/mL in T cell culture media supplemented with Th1 skewing cytokines and 1 g/mL soluble αCD28. Cells were refed on day 3 with half of the initial volume of T cell culture media and twice the concentration of cytokines. On day 7, the frequency and number of OT-II and SMART-A1 T cells were determined by harvesting and counting samples, staining them with tetramers or PE anti-mouse CD45.1 clone A20 (Biolegend) antibodies, respectively, and analyzing them on a BD FACSCalibur flow cytometer. Endogenous murine CD4⁺ T cell enrichment and expansion studies were performed analogously to the doped enrichment and expansion studies, using freshly isolated B6 CD4⁺ T cells. On day 7, the frequency and number of antigen-specific CD4⁺ T cells was determined by harvesting and counting samples, staining them with cognate and non-cognate tetramers, and then analyzing them on a BD FACSCalibur flow cytometer. Endogenous murine CD8⁺ T cell enrichment and expansion studies were performed as previously described, Hickey et al., 2020, by isolating B6 CD8⁺ T cells, and then incubating them for 1 hour with MHC I aAPCs (30 ng conjugated K^{b}-Ig or D^{b}-Ig per 106 CD8⁺ T cells) at 4° C. in AutoMACS Running Buffer (1× PBS with 2 mM EDTA and 0.5% Bovine Serum Albumin). Cells were then magnetically enriched on a 96-well ring magnet and plated at 2.5×10⁵ cells/mL in T cell culture media supplemented with an optimized CD8⁺ cytokine mix, Oelke et al., 2000, and 1 g/mL soluble αCD28. For endogenous co-culture experiments, the enriched fractions were additionally supplemented with an equal number of Day 5 Th1 skewed CD4⁺ T cells (see above) and S1 aAPCs (80 ng/mL conjugated I-Ab). Cells were refed on day 3 with half of the initial volume of T cell culture media and twice the concentration of the CD8⁺ cytokine mix. On day 7, cells were harvested and counted, and then analyzed for specificity, phenotype, and function of dimer positive CD8⁺ T cells. Representative gating strategies for binding, internalization, and enrichment experiments can be found in FIG. 14a.

### 1.5.16 Imaging Studies

OT-I/OT-II imaging studies were performed by labeling freshly isolated OT-I CD8+ T cells at 37° C. for 20 minutes with 5 μM CellTracker green dye (Invitrogen) in T cell culture media without serum and then quenching at 37° C. for 5 additional minutes with 5 mL FBS. Analogously, freshly isolated or Day 5 Th1 skewed OT-II CD4⁺ T cells were labeled with 5 μM CellTrace Far Red dye (Invitrogen). Labeled OT-II CD4⁺ T cells were then pre-incubated with MHC I/II aAPCs at 80 ng conjugated I-A^{b}/10⁵ CD4⁺ T cells for two hours at 37° C., prior to mixing them 1:1 with labeled OT-I CD8⁺ T cells. T cell mixtures were incubated on gelatin coated (0.1%) plates and imaged using a Zeiss AxioObserver epifluorescent microscope with an incubation chamber at 37° C. and 5% CO₂. Images at 24 hours were analyzed using a custom protocol in CellProfiler. CD4⁺ and CD8⁺ T cells within 5 pixels of each other were considered bound. OT-II internalization imaging studies were performed by incubating freshly isolated OT-II CD4⁺ T cells with nanoparticles conjugated with Alexa Fluor 488-labelled I-A_{OVA}^{b} tetramer at a concentration of 80 ng I-A^{b}/10⁶ cells for 2 hours at 37° C. Cells were then washed in PBS and stained with Alexa Fluor 594 anti-mouse CD4 clone GK1.5 (BioLegend) and Alexa Fluor 647 anti-mouse I-A/I-E clone M5/114.15.2 (BioLegend) antibodies for 15 minutes at 4° C. Cells were then washed in PBS and fixed overnight in 1% paraformaldehyde. The following morning, cells were washed in PBS and stained with DAPI (ThermoFisher) at 0.1 μg/mL for 10 minutes at 20° C. Cells were then washed and imaged in a #1.5 chambered coverglass slide (Cellvis) using an LSM980 confocal microscope with Airyscan super-resolution. Airyscan processing was performing using Zen software, and the Pearson Correlation between Alexa Fluor 488 and Alexa Fluor 647 fluorescent signals was calculated in ImageJ.

### 1.5.17 Transwell Migration Assays

Transwell migration assays were performed as previously described, Galeano Niño et al., 2020, using transwell plates (Costar) with 5.0 μm pore-size polycarbonate membranes. Day 7 stimulated OT-I CD8⁺ T cells were labelled at 37° C. for 20 minutes with 5 M CFSE dye (Invitrogen) in T cell culture media without serum and then quenched at 37° C. for 5 additional minutes with 5 mL FBS. Analogously, freshly isolated or Day 5 Th1 skewed OT-II CD4⁺ T cells were labeled with 5 M CellTrace Far Red dye (Invitrogen). The bottom compartments of the transwell plates received 600 μL of control medium (RPMI 1640 with 0.5% BSA) with or without 1×10⁶ labelled naive or Th1 OT-II CD4⁺ T cells at a 1:1 ratio with αCD3/αCD28 Dynal microbeads, while the top compartments received 1×10⁶ OT-I CD8⁺ T cells in 100 μL control medium. Plates were incubated at 37° C. for 3 hours and then the upper and lower compartments were harvested, manually counted with a hemocytometer, and stained with Live/Dead Fixable Violet (Invitrogen), PE anti-mouse CD4 clone H129.19 (BioLegend), and PE/Cyanine7 anti-mouse CD8 clone 53-6.7 (BD Biosciences). Cells were washed, resuspended in FACS Wash Buffer and analyzed on an Attune N×T Flow Cytometer. The transmigration index was calculated as the ratio of the number of CD8⁺ T cells transmigrated in a given sample to the number of CD8⁺ T cells transmigrated in control medium.

### 1.5.18 Protein Arrays

Day 5 Th1 OT-II CD4⁺ T cells were either left unstimulated or were re-stimulated overnight with MHC II aAPCs (80 ng I-Ab/10⁵ CD4⁺ T cells) and soluble αCD28 (1 g/10⁵ CD4⁺ T cells). Cell supernatants were then collected and filtered through Spin-X Centrifuge Tube filters (Corning). Cytokines in the cell supernatants were then analyzed with the Proteome Profiler Mouse Cytokine Array Kit A (R&D Systems). The blots were visualized with chemiluminescence using an iBright 1500 imaging system and quantified using the Protein Array Analyzer plugin in ImageJ.

1.5.19 Cloning of HA1.7 TCR The native signal sequence and a and R variable domains of TCR HA1.7, Hennecke et al., 2000, (IMGT ID 1FYT) were cloned into the AbVec mammalian expression vector, Wagner et al., 2019, containing the murine constant domains—to promote pairing of the exogenous α and β TCR chains- and human transmembrane domains (see Table 2). The a and R chains were separated by a P2A peptide. Plasmid was purified using ZymoPURE II Plasmid Midiprep Kit (Zymo Research).

### 1.5.20 HA1.7 Expression and Activation in Jurkat Cells

10⁷ Jurkat cells per transfection were centrifuged at 250×g for 5 minutes, resuspended in 5 mL of OptiMEM (Gibco), and incubated at 20° C. for 8 minutes. Cells were centrifuged as before, resuspended in 400 μl of OptiMEM and 20 μg HA1.7 plasmid, and transferred to a 4-mm electroporation cuvette (BioRad). Cells were incubated for 8 minutes before pulsing exponentially with 250 V. 950 μF, and ∞ ohms resistance on a Bio-Rad GenePulser Xcell with PC and CE modules. After an 8 minute recovery period, cells were rescued with 10 mL of pre-warmed Jurkat culture media (RPMI 1640+10% FBS+100 U/mL penicillin-streptomycin), and kept at 37° C., 5% CO₂. In vitro stimulation of HA1.7 TCR-transfected Jurkat cells was performed 12-16 hours after transfection. αCD3/αCD28 microbeads or titrations of nanoscale DR1 HA peptide exchanged and DR1 CLIP unexchanged aAPCs were incubated at 37° C. with 5×10⁴ transfected Jurkat T cells per stimulation in Jurkat culture media. At 24 hours post-transfection, samples were washed and stained for 15 minutes at 4° C. in FACS Wash Buffer with APC anti-mouse TCR R chain clone H57-597 (BioLegend) and FITC anti-human CD69 clone FN50 (BioLegend) to detect the HA1.7 TCR and activation, respectively. Cells were then washed again and analyzed on a BD FACSCalibur Flow Cytometer.

### 1.5.21 In Vivo Killing Assay

One day prior to adoptive cell transfer (ACT), CD45.1 B6 mice received 500 cGy of irradiation to induce transient lymphopenia and promote T cell engraftment. Wrzesinski et al., 2010. On the day of adoptive transfer, OT-II CD4⁺ T cells were either freshy isolated (naïve) or harvested after 7 days of stimulation with MHC II aAPCs (80 ng/mL conjugated I-A^{b} and 1 μg/mL soluble αCD28) in Th1 skewing media (Th1). Naive and Th1 CD4⁺ T cells were labeled with 5 μM CellTrace Violet (CTV, Invitrogen) in 1 mL PBS for 20 minutes at 37° C. The reaction was quenched with 5 mL FBS at 37° C. for 5 minutes, and then cells were washed twice in PBS. 10⁶ CTV labelled naive or Th1 CD4⁺ T cells were then injected intravenously in volumes of 100 L per recipient mouse. On the day of and the day after adoptive transfer, mice received intraperitoneal injections of 30,000 U IL-2 (Prometheus Labs) in a volume of 100 μL. To analyze in vivo killing, six days post adoptive transfer, freshly isolated spleens from B6 mice were brought to a single cell suspension. Cells were then labeled either with 5 μM or 0.5 μM CFSE (Invitrogen) to generate CFSE^{h}i and CFSE^{lo} populations. CFSE^{hi} splenocytes were then loaded for 1 hour at 37° C. with 1 μg of OVA₃₂₃₋₃₃₉ peptide per 10⁷ cells in T cell culture media, washed twice in PBS, and mixed 1:1 with unloaded CFSE^{lo} splenocytes. 10⁷ cells of the mixture were then injected intravenously in 100 L volumes per recipient mouse. The following day, spleens and lymph nodes of recipient mice were harvested, processed, and stained for Live/Dead Fixable Aqua (Invitrogen), PE anti-mouse CD45.2 clone 104 (BioLegend), APC anti-mouse CD4 clone GK1.5 (BioLegend), and PE/Cyanine7 anti-mouse I-A/I-E clone M5/114.15.2 (BioLegend) for 15 minutes at 4° C. Cells were then washed, resuspended in FACS Wash Buffer, and analyzed on an Attune N×T Flow Cytometer. Specific lysis was calculated as 100%×(1−[(CFSE^{lo,pre-injection}/CFSE^{hi,pre-injection)}/(CFSE^{loPost-injection}/CFSE^{hi,post-injection})]). Representative gating strategies for in vivo killing assays can be found in FIG. 14c. To analyze in vivo phenotypic and functional markers, 21 days post adoptive transfer, spleens and lymph nodes were harvested from recipient mice, processed, resuspended at 10⁷ cells/mL in T cell culture media and incubated at 37° C. for 6 hours with 1× cytokine activation cocktail (BioLegend) and GolgiPlug (BD Biosciences). No stimulation controls received only GolgiPlug. Following incubation, cells were washed and stained with Live/Dead Fixable Aqua (Invitrogen), PE anti-mouse CD45.2 clone 104 (BioLegend), and APC anti-mouse CD4 clone GK1.5 (BioLegend). Cells were then washed, fixed and permeabilized overnight with the Foxp3 Transcription Factor Staining Buffer Set (eBioscience), and then stained with APC anti-mouse IFN-γ clone XMG1.2 (BioLegend), PE/Cyanine7 anti-mouse TNF-α clone MP6-XT22 (BioLegend), and PerCp-Cyanine5.5 anti-mouse/human T-bet clone eBio4B10 (eBioscience) or its corresponding isotype. Finally, cells were washed and resuspended in FACS wash buffer (1× PBS, 2% FBS, 0.5% sodium azide) and then analyzed on an Attune N×T Flow Cytometer.

### 1.5.22 Adoptive Transfer Melanoma Model

The in vivo therapeutic efficacy of OT-I CD8′ T cells co-cultured with Th1 OT-1I CD4⁺ T cells was compared to traditionally stimulated OT-I CD8⁺ T cells using a B16-OVA murine melanoma model. On day 0, B6 mice received a subcutaneous injection of 2×10⁵ tumor cells on the left flank. On that same day, OT-II CD4⁺ T cells were activated in Th1 skewing media with MHC II aAPCs (80 ng/mL conjugated I-A^{b} and 1 g/mL soluble αCD28). On day 5, OT-I CD8⁺ T cells were stimulated with MHC I aAPCs (30 ng/mL conjugated K^{b} and 1 g/mL soluble αCD28) in T cell culture media supplemented with TF. Co-cultured OT-I CD8⁺ T cells were additionally mixed at a 1:1 ratio with the day 5 Th1 OT-II CD4⁺ T cells and MHC II aAPCs (80 ng/mL conjugated I-A^{b}). On day 10, 2×10⁶ OT-I CD8⁺ T cells that were freshly isolated, stimulated alone, or stimulated in co-culture with Th1 OT-II CD4⁺ T cells, were injected intravenously in 100 L volumes into B16-OVA tumor bearing mice. On the day of and the day after adoptive transfer, mice received intraperitoneal injections of 30,000 U IL-2 (Prometheus Labs) in 100 μL volumes. Tumor size was measured with digital calipers every 2-3 days until tumors became necrotic or reached 200 mm², after which mice were sacrificed with CO₂ asphyxiation and cervical dislocation.

1.5.23 Statistical Analysis Error bars in graphs represent the standard error of the mean (s.e.m.) unless otherwise stated. All n values are given in the Figure legends. Statistical analyses were performed in GraphPad Prism software version 8.4.3. Two-tailed Student's t tests were used for comparisons between two groups. One and two-way ANOVAs with Tukey's multiple comparisons test were used for comparisons between multiple groups. One-way ANOVAs with Dunnett's post-hoc test was used for comparison of multiple groups to a control group. Repeated measure two-way ANOVAs with Tukey's multiple comparisons test were used for comparing tumor growth curves, and log-rank tests were used for comparing survival curves.

### Example 2

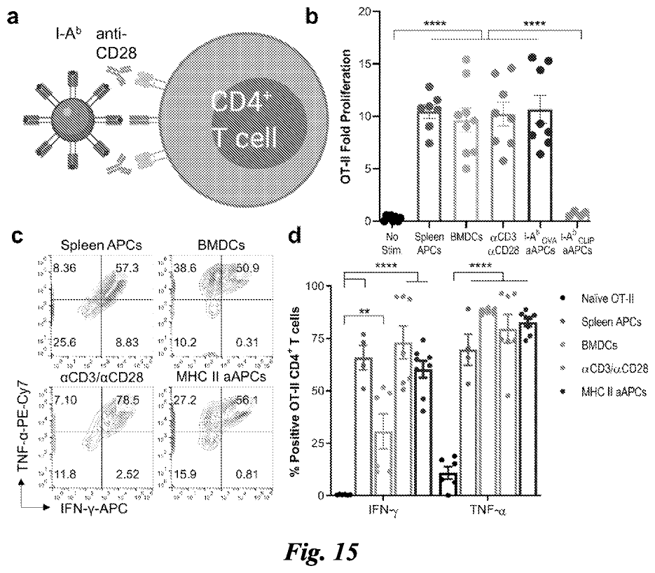

In further embodiments, we synthesized MHC II aAPCs, 300 nm iron dextran nanoparticles conjugated with peptide-loaded I-A^{b} molecules as S1, while anti-CD28 as S2 was added into solution during T cell culture (FIG. 15a). MHC II aAPC stimulation of OT-II CD4⁺ T cells cultured in a Th1 skewing mix (IL-2, IL-12, and IFN-γ), led to similar levels of antigen-specific CD4⁺ T cell expansion and function as traditional approaches, such as αCD3/αCD28 microparticles, spleen APCs, or bone marrow derived DCs (BMDCs) (FIG. 15b-c).

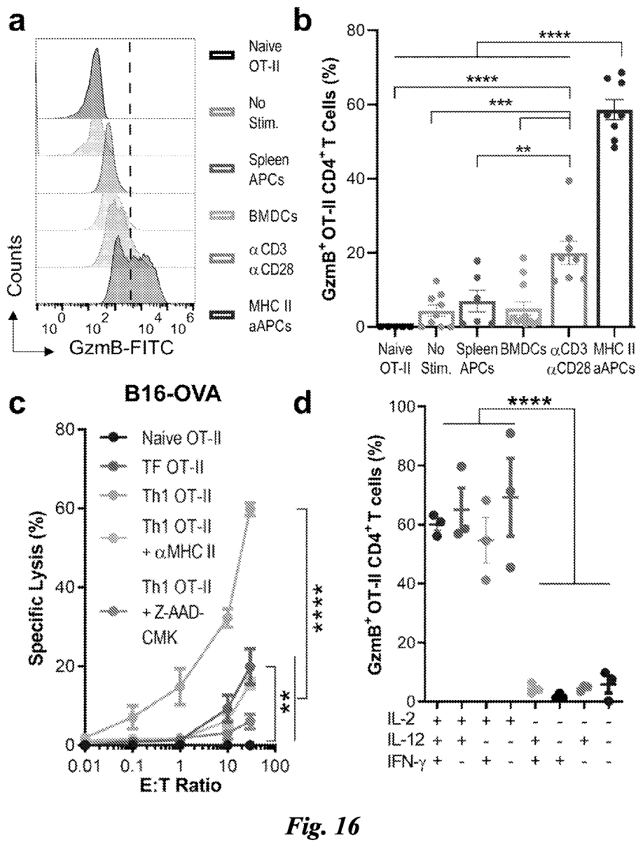

Interestingly, unlike these other approaches, aAPCs uniquely induced CD4⁺ CTL differentiation, as observed through upregulation of Granzyme B (GzmB) (FIG. 16a-b) and specific-lysis of cognate B16-OVA tumor cells in an MHC II− and GzmB-dependent manner (FIG. 16c). The cytotoxicity of aAPC activated CD4⁺ T cells also depended on the cytokine milieu; T cell growth factor media (TF) generated significantly fewer lytic CD4⁺ T cells (FIG. 16c) than Th1 media.

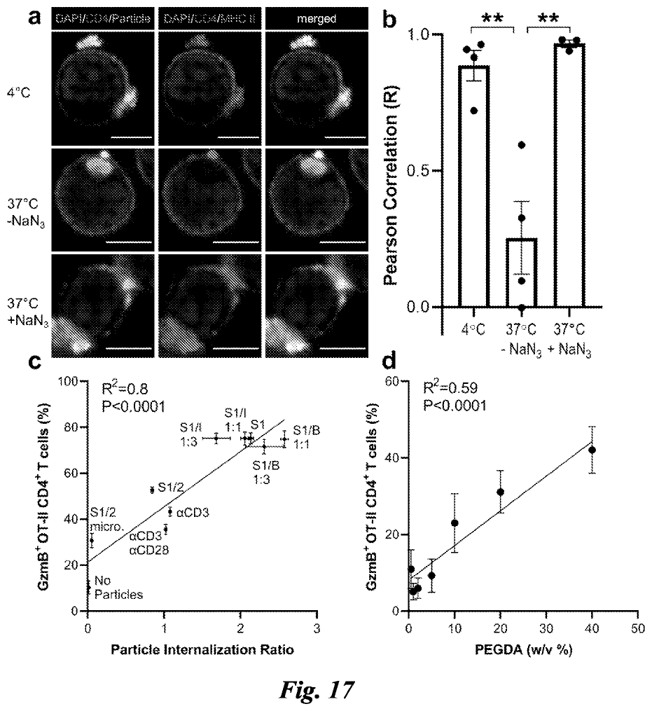

Closer analysis of the Th1 cytokines revealed that, specifically, IL-2 was necessary for CD4⁺ CTL induction (FIG. 16d). We had separately observed profound TCR-mediated internalization of MHC II aAPCs by OT-II CD4⁺ T cells after incubation at 37° C. for two or more hours (FIG. 17a-b); hence we asked whether particle internalization was contributing to CTL induction.

Interestingly, the extent of internalization for a variety of particles, as measured by the loss of surface-bound particles between 30 and 120 minutes, was tightly correlated with OT-II day 7 GzmB levels (FIG. 17c). This, together with our observation that hyaluronic acid hydrogel substrate rigidity impacts GzmB levels (FIG. 17d), suggested that mechanical cues delivered to the cells could be important for CD4⁺ CTL induction.

Referring now to FIG. 17, correlation between uptake and granzyme induction is very tight, FIG. 17C. All of the particles used in FIG. 17C were matched for amounts of what we call Signal 1 which was either cognate antigen (I-Av) or anti-CD3. I-A^{b} performed significantly better than anti-CD3 although they both have the same amount of protein. Thus there is something unexpectedly different about the way antigen, I-Ab, binds TCR than CD3. Signal 2, anti-CD28, seems to block uptake, S1/2 versus S1/1 or S1 or S1/B, FIG. 17C. FIG. 17D shows this is dependent on stiffness of the particle- also novel and unpredicted. FIG. 17B shows it's a metabolically active process. All these points are relevant to the novelty of activation by the class II-based aAPC, original application, and also relevant to the new application of using this approach to modify T cells, either genetically or otherwise, to alter T cell function or fate. Also while documented for CD4⁺ cells could also be true for CD8⁺ T cells.

### Example 3

### Production and Testing of DR-Fc aAPCs

Further, aAPCs comprising DR-Fc fusions were designed, produced, and tested to evaluate stimulation of human T cells by DR-Fc aAPCs.

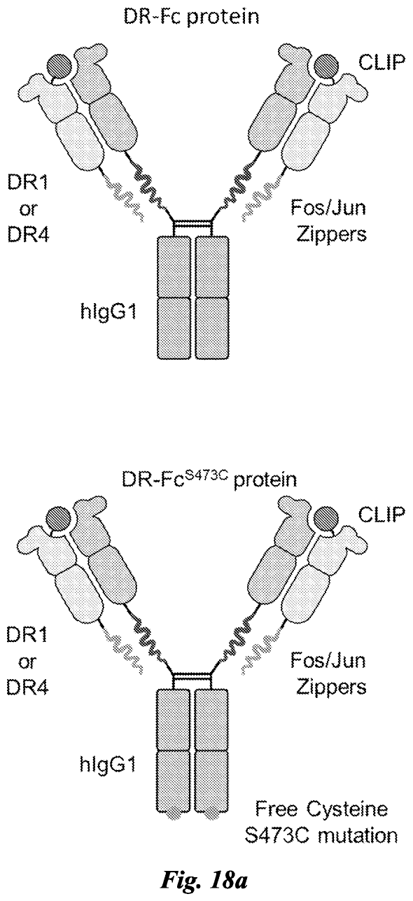

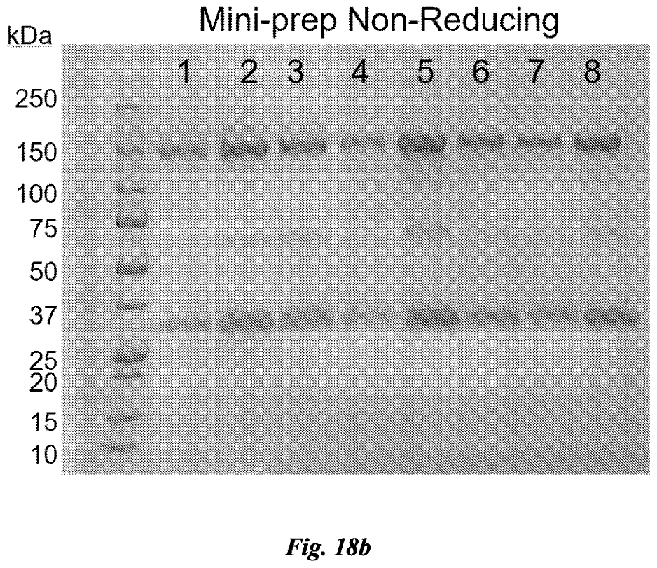

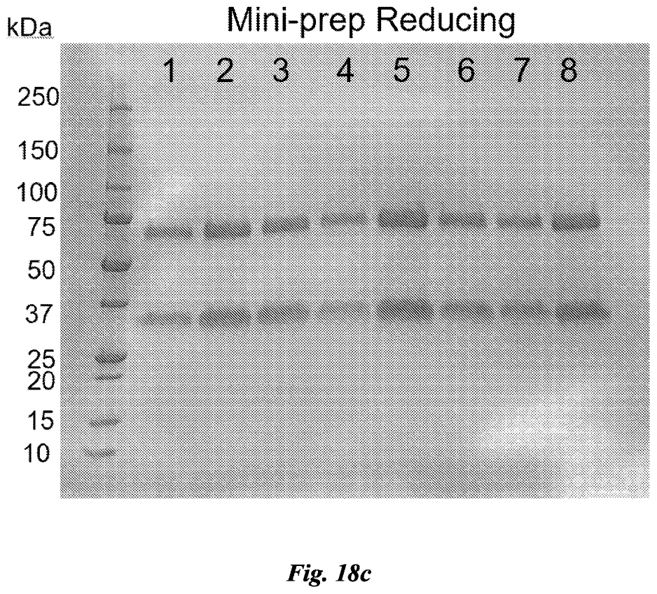

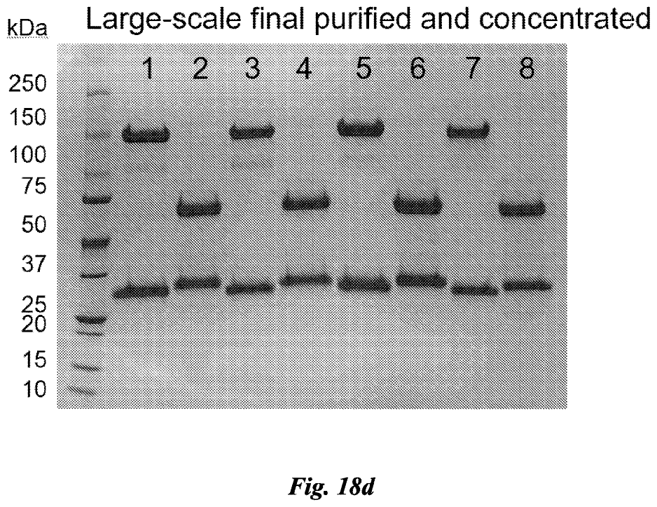

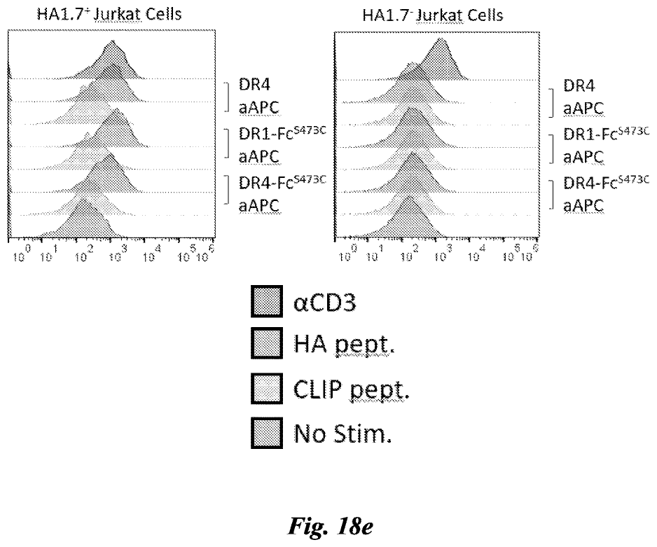

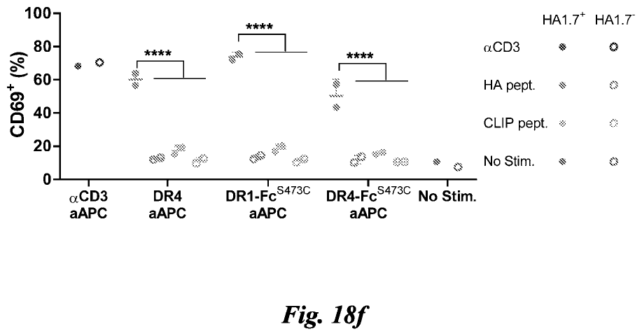

DR-Fc fusions proteins were designed as described using the methods described herein to produce a cysteine substituted and cysteine non-substituted constructs shown in FIG. 18a. In particular, the DR-Fc fusions were designed to comprise a human IgG1 Fc domain (hIgG1), a DR1 or DR4 chain, Fos/Jun zipper domains, and a CLIP chain peptide (FIG. 18a). DR1-Fc and DR4-Fc proteins were produced comprising an unmodified Fe sequence (FIG. 18a, top) and comprising a substitution of cysteine for the serine at position 473 (S473C) (FIG. 18a, bottom).

The DR1-Fc and DR4-Fc fusions were expressed recombinantly using methods described herein to co-transfect plasmids encoding the respective DRα and DRβ chains. DRα and DRβ chain plasmids were titrated in small-scale co-transfection tests to determine optimal DNA ratios (1:2α:β, 1:1 α:β, and 2:1α:β; FIG. 18b and FIG. 18c) for large-scale expression and purification (FIG. 18d). Small-scale protein preparations were produced under non-reducing (FIG. 18b) and reducing (FIG. 18c) conditions, and products were assayed by gel electrophoresis. The large-scale purified and concentrated proteins were produced under reducing and non-reducing conditions and verified by gel electrophoresis (FIG. 18d). The results indicated successful expression and purification of the DR1-Fc and DR4-Fc cysteine substituted and cysteine non-substituted fusion proteins.

The DR4, DR1-Fc^{S473C}, and DR4-Fc^{S473C} proteins were used to produce DR4, DR1-FC^{S473C}, and DR4-Fc^{S473C} aAPCs using methods described herein. Jurkat cells were transfected with the HA1.7 TCR overnight using methods described herein. Non-transfected cells were used as a control. The CLIP peptides of the DR4, DR1-Fc^{S473C}, and DR4-Fc^{S473C} constructs were peptide exchanged with HA peptide overnight using methods as described herein. The transfected Jurkat T cells were stimulated for 6 hours with peptide exchanged aAPCs, peptide unexchanged aAPCs, and polyclonal anti-CD3. Non-stimulated Jurkat T cells were used as a control. CD69 expression after stimulation was measured using flow cytometry (FIG. 18e and FIG. 18f). As indicated by the data shown in FIG. 18e, stimulation of the HA1.7+Jurkat cells with the DR4 aAPC and with the DR1-Fc^{S473C} and DR4-Fc^{S473C} fusion construct aAPCs induced the CD69 marker in more cells than in HA1.7* Jurkat cells stimulated with unexchanged constructs comprising the CLIP peptide. The stimulation was similar to stimulation produced by anti-CH3 antibody in both HA1.7* and HA1.7- Jurkat cells. DR4 aAPC, DR1-Fc^{S473C} aAPC, and DR4-Fc^{S473C} aAPCs did not stimulate HA1.7-Jurkat cells. Stimulation was quantified by calculating the percentage of HA1.7* and HA1.7-cells expressing CD69 (FIG. 18f).

In sum, these data indicate that DR1-Fe, DR4-Fc, DR1-Fc^{S473C}, and DR4-Fc^{S473C}proteins were successfully expressed. Further, DR1-Fc^{S473C} and DR4-Fc^{S473C} aAPCs stimulated cognate Jurkat cells with antigen-specificity similarly to stimulation observed for DR4 aAPCs. In contrast, the anti-CD3 antibody non-specifically activates cognate and non-cognate Jurkat cells.

### Example 4

### Nanoparticles For Delivery Of Immunoregulatory Materials To T Cells

Helper (CD4⁺) T cells perform direct therapeutic functions and augment responses of cells, such as cytotoxic (CD8⁺) T cells, against a wide variety of diseases and pathogens. Nevertheless, inefficient synthetic technologies for expansion of antigen-specific CD4⁺ T cells hinders consistency and scalability of CD4⁺ T cell-based therapies, and complicates mechanistic studies.

Here we describe a nanoparticle platform for ex vivo CD4⁺ T cell culture that mimics antigen presenting cells (APC) through display of major histocompatibility class II (MHC II) molecules. When combined with soluble co-stimulation signals, MHC II artificial APCs (aAPC) expand cognate murine CD4⁺ T cells, including rare endogenous subsets, to induce potent effector functions in vitro and in vivo.

Moreover, MHC II aAPCs provide help signals that enhance antitumor function of aAPC-activated CD8⁺ T cells in a mouse tumor model. Lastly, human leukocyte antigen class II-based aAPCs expand rare subsets of functional, antigen-specific human CD4⁺ T cells. Overall. MHC II aAPCs provide a promising approach for harnessing targeted CD4⁺ T cell responses.

One of our novel findings is that MHC II-based aAPC are internalized by their cognate T cells. Thus, MHC II-based aAPC approach may represent a novel, non-viral, approach to delivering immunomodulatory materials, including genetic material, to T cells including CD4⁺ T cells.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Grupp, S. A. et al. Chimeric antigen receptor-modified T cells for acute lymphoid leukemia. N. Engl. J. Med. 368, 1509-1518 (2013).
Porter, D. L., Levine, B. L., Kalos, M., Bagg, A. & June, C. H. Chimeric Antigen Receptor-Modified T Cells in Chronic Lymphoid Leukemia. N. Engl. J. Med. 365, 725-733 (2011).
Tran, E. et al. Cancer Immunotherapy Based on Mutation-Specific CD4⁺ T Cells in a Patient with Epithelial Cancer. Science (80-. ). 344, 641 LP—645 (2014).
Hunder, N. N. et al. Treatment of metastatic melanoma with autologous CD4⁺ T cells against NY-ESO-1. N. Engl. J. Med. 358, 2698-2703 (2008).
Isser, A., Livingston, N. K. & Schneck, J. P. Biomaterials to enhance antigen-specific T cell expansion for cancer immunotherapy. Bioniaterials 268, 120584 (2021).
Oelke, M. et al. Ex vivo induction and expansion of antigen-specific cytotoxic T cells by HLA-Ig-coated artificial antigen-presenting cells. Nat. Med. 9, 619-625 (2003).
Kreiter, S. et al. Mutant MHC class II epitopes drive therapeutic immune responses to cancer. Nature 520, 692-6 (2015).
Alspach, E. et al. MHC-II neoantigens shape tumour immunity and response to immunotherapy. Nature 574, 696-701 (2019).
Mumberg, D. et al. CD4⁺ T cells eliminate MHC class II-negative cancer cells in vivo by indirect effects of IFN-γ. Proc. Natl. Acad. Sci. 96, 8633 LP—8638 (1999).
Hung, K. et al. The Central Role of CD4(+) T Cells in the Antitumor Immune Response. J. Exp. Med. 188, 2357-2368 (1998).
Perez-diez, A. et al. CD4 cells can be more efficient at tumor rejection than CD8 cells. Blood 109, 5346-5355 (2016).
Quezada, S. A. et al. Tumor-reactive CD4(+) T cells develop cytotoxic activity and eradicate large established melanoma after transfer into lymphopenic hosts. J. Exp. Med. 207, 637-650 (2010).
Borst, J., Ahrends, T., Bgbala, N., Melief, C. J. M. & Kastenmuller, W. CD4⁺ T cell help in cancer immunology and immunotherapy. Nat. Rev. Immunol. 18, 635-647 (2018).
Sugata, K. et al. Affinity-matured HLA class II dimers for robust staining of antigen-specific CD4⁺ T cells. Nat. Biotechnol. 39, 958-967 (2021).
Lillemeier, B. F. et al. TCR and Lat are expressed on separate protein islands on T cell membranes and concatenate during activation. Nat. Immunol. 11, 90-96 (2010).
Hickey, J. W., Vicente, F. P., Howard, G. P.. Mao, H.-Q. & Schneck, J. P. Biologically Inspired Design of Nanoparticle Artificial Antigen-Presenting Cells for Immunomodulation. Nano Lett. 17, 7045-7054 (2017).
Tay, R. E., Richardson, E. K. & Toh, H. C. Revisiting the role of CD4⁺ T cells in cancer immunotherapy-new insights into old paradigms. Cancer Gene Ther. 28, 5-17 (2021).
Oelke, M. et al. Generation and purification of CD8⁺ melan-A-specific cytotoxic T lymphocytes for adoptive transfer in tumor immunotherapy. Clin. Cancer Res. 6, 1997-2005 (2000).
Perica, K. et al. Enrichment and Expansion with Nanoscale Artificial Antigen Presenting Cells for Adoptive Immunotherapy. ACS Nano 9, 6861-6871 (2015).
Hickey, J. W. et al. Efficient magnetic enrichment of antigen-specific T cells by engineering particle properties. Biomaterials 187, 105-116 (2018).
Ichikawa, J. et al. Rapid Expansion of Highly Functional Antigen-Specific T Cells from Patients with Melanoma by Nanoscale Artificial Antigen-Presenting Cells. Clin. Cancer Res. 26, 3384-3396 (2020).
Cameron, T. O., Cochran, J. R., Yassine-Diab, B., Sêkaly, R.-P. & Stern, L. J. Cutting Edge: Detection of Antigen-Specific CD4⁺ T Cells by HLA-DR1 Oligomers Is Dependent on the T Cell Activation State. J. Immunol. 166, 741 LP—745 (2001).
Hickey, J. W. et al. Adaptive Nanoparticle Platforms for High Throughput Expansion and Detection of Antigen-Specific T cells. Nano Lett. 20, 6289-6298 (2020).
Moon, J. J. et al. Naive CD4(+) T cell frequency varies for different epitopes and predicts repertoire diversity and response magnitude. Immunity 27, 203-213 (2007).
Oh, D. Y. et al. Intratumoral CD4(+) T Cells Mediate Anti-tumor Cytotoxicity in Human Bladder Cancer. Cell 181, 1612-1625.e13 (2020).
Cachot, A. et al. Tumor-specific cytolytic CD4 T cells mediate immunity against human cancer. Sci. Adv. 7, eabe3348 (2021).
Melenhorst, J. J. et al. Decade-long leukaemia remissions with persistence of CD4⁺ CAR T cells. Nature 602, 503-509 (2022).
Kaluza, K. M. et al. Adoptive T cell therapy promotes the emergence of genomically altered tumor escape variants. Int. J. Cancer 131, 844-854 (2012).
Day, C. L. et al. Ex vivo analysis of human memory CD4 T cells specific for hepatitis C virus using MHC class II tetramers. J. Clin. Invest. 112, 831-842 (2003).
Hickey, J. W. et al. Engineering an Artificial T-Cell Stimulating Matrix for Immunotherapy. Adv. Mater. 31, 1807359 (2019).
Cheung, A. S., Zhang, D. K. Y. Y., Koshy, S. T. & Mooney, D. J. Scaffolds that mimic antigen-presenting cells enable ex vivo expansion of primary T cells. Nat. Biotechnol. 36, 160-169 (2018).
Rhodes, K. R. et al. Biodegradable Cationic Polymer Blends for Fabrication of Enhanced Artificial Antigen Presenting Cells to Treat Melanoma. ACS Appl. Mater. Interfaces 13, 7913-7923 (2021).
Fadel, T. R. et al. A carbon nanotube-polymer composite for T-cell therapy. Nat. Nanotechnol. 9, 639-647 (2014).
Zander, R. et al. CD4(*) T Cell Help Is Required for the Formation of a Cytolytic CD8(+) T Cell Subset that Protects against Chronic Infection and Cancer. Immunity 51, 1028-1042.e4 (2019).
Clemente-Casares, X. et al. Expanding antigen-specific regulatory networks to treat autoimmunity. Nature 530, 434-440 (2016).
Singha, S. et al. Peptide-MHC-based nanomedicines for autoimmunity function as T-cell receptor microclustering devices. Nat. Nanotechnol. 12, 701-710 (2017).
Maus, M. V et al. Ex vivo expansion of polyclonal and antigen-specific cytotoxic T lymphocytes by artificial APCs expressing ligands for the T-cell receptor, CD28 and 4-1BB. Nat. Biotechnol. 20, 143-148 (2002).
Wölfl, M. & Greenberg, P. D. Antigen-specific activation and cytokine-facilitated expansion of naive, human CD8⁺ T cells. Nat. Protoc. 9, 950-966 (2014).
Gigante, M. et al. Dysfunctional DC subsets in RCC patients: ex vivo correction to yield an effective anti-cancer vaccine. Mol. Immunol. 46, 893-901 (2009).
Satthaporn, S. et al. Dendritic cells are dysfunctional in patients with operable breast cancer. Cancer Immunol. Immunother. 53, 510-8 (2004).
Wculek, S. K. et al. Dendritic cells in cancer immunology and immunotherapy. Nat. Rev. Immunol. 20, 7-24 (2020).
Sledzinska, A. et al. Regulatory T Cells Restrain Interleukin-2- and Blimp-1-Dependent Acquisition of Cytotoxic Function by CD4⁺ T Cells. Immunity 52, 151-166.e6 (2020).
Dennis, K. L., Blatner, N. R., Gounari, F. & Khazaie, K. Current status of interleukin-10 and regulatory T-cells in cancer. Curr. Opin. Oncol. 25, 637-645 (2013).
Steinbrink, K., Graulich, E., Kubsch, S., Knop, J. & Enk, A. H. CD4⁺ and CD8* anergic T cells induced by interleukin-10-treated human dendritic cells display antigen-specific suppressor activity. Blood 99, 2468-2476 (2002).
Groux, H., Bigler, M., de Vries, J. E. & Roncarolo, M.-G. Inhibitory and Stimulatory Effects of IL-10 on Human CD8⁺ T Cells. J. Immunol. 160, 3188 LP—3193 (1998).
Naing, A. et al. PEGylated IL-10 (Pegilodecakin) Induces Systemic Immune Activation, CD8⁺ T Cell Invigoration and Polyclonal T Cell Expansion in Cancer Patients. Cancer Cell 34, 775-791.e3 (2018).
Emmerich, J. et al. IL-10 Directly Activates and Expands Tumor-Resident CD8⁺ T Cells without De Novo Infiltration from Secondary Lymphoid Organs. Cancer Res. 72, 3570 LP—3581 (2012).
Mumm, J. B. et al. IL-10 Elicits IFNy-Dependent Tumor Immune Surveillance. Cancer Cell 20, 781-796 (2011).
Saxton, R. A. et al. Structure-based decoupling of the pro- and anti-inflammatory functions of interleukin-10. Science (80-. ). 371, eabc8433 (2021).
Sha, W. C. et al. Selective expression of an antigen receptor on CD8-bearing T lymphocytes in transgenic mice. Nature 335, 271-274 (1988).
Drover, S., Karr, R. W., Fu, X.-T. & Marshall, W. H. Analysis of monoclonal antibodies specific for unique and shared determinants on HLA-DR4 molecules. Hum. Immunol. 40, 51-60 (1994).
Kosmides, A. K., Sidhom, J.-W., Fraser, A., Bessell, C. A. & Schneck, J. P. Dual Targeting Nanoparticle Stimulates the Immune System To Inhibit Tumor Growth. ACS Nano 11, 5417-5429 (2017).
Lutz, M. B. et al. An advanced culture method for generating large quantities of highly pure dendritic cells from mouse bone marrow. J. Immunol. Methods 223, 77-92 (1999).
Galeano Nino, J. L. et al. Cytotoxic T cells swarm by homotypic chemokine signalling. Elife 9, e56554 (2020).
Hennecke, J., Carfi, A. & Wiley, D. C. Structure of a covalently stabilized complex of a human alphabeta T-cell receptor, influenza HA peptide and MHC class II molecule, HLA-DR1. EMBO J. 19, 5611-5624 (2000).
Wagner, E. K. et al. Human cytomegalovirus-specific T-cell receptor engineered for high affinity and soluble expression using mammalian cell display. J. Biol. Chem. 294, 5790-5804 (2019).
Wrzesinski, C. et al. Increased intensity lymphodepletion enhances tumor treatment efficacy of adoptively transferred tumor-specific T cells. J. Immunother. (Hagerstown, Md. 1997) 33, 1 (2010).

Although the foregoing subject matter has been described in some detail by way of illustration and example for purposes of clarity of understanding, it will be understood by those skilled in the art that certain changes and modifications can be practiced within the scope of the appended claims.

1. An artificial antigen presenting cell (aAPC) comprising a particle having a major histocompatibility complex class II (MHC II) molecule conjugated to a surface thereof.

2. An artificial antigen presenting cell (aAPC) consisting essentially of a particle having a major histocompatibility complex class II (MHC II) molecule conjugated to a surface thereof.

3. The aAPC of claim 1 or claim 2, wherein the MHC II molecule comprises an MHC II I-A^{b} monomer.

4. The aAPC of any one of claims 1-3, further comprising a costimulatory ligand conjugated to a surface thereof.

5. The aAPC of claim 4, wherein the costimulatory ligand is selected from the group consisting of an antibody or antigen-binding fragment thereof that specifically binds to CD28, CD80 (B7-1), CD86 (B7-2), B7- H3, 4-1BBL, 4-11B, CD27, CD30, CD134 (OX-40L), B7h (B7RP-1), CD40, LIGHT, an antibody or antigen-binding fragment thereof that specifically binds to HVEM, an antibody or antigen-binding fragment thereof that specifically binds to CD40L, an antibody or antigen binding fragment thereof that specifically binds to OX40, and an antibody or antigen-binding fragment thereof that specifically binds to 4-1BB.

6. The aAPC of claim 5, wherein the costimulatory ligand comprises an anti-CD28 (αCD28) antibody.

7. The aAPC of any one of claims 1-3, further comprising a major histocompatibility complex class I molecule conjugated to a surface thereof.

8. The aAPC of claim 7, wherein the MHC-class I molecule comprises a K^{b}-Ig dimer.

9. The aAPC of claim 1 or claim 2, wherein the MHC II molecule comprises a human leukocyte antigen (HLA) class II monomer.

10. The aAPC of claim 9, wherein the HLA class II monomer is selected from the group consisting of HLA-DR, HLA-DP, and HLA-DQ.

11. The aAPC of claim 10, wherein the HLA class II monomer comprises DR1 or DR4.

12. The aAPC of claim 9, wherein the HLA class II monomer comprises a cleavable thrombin linker, wherein the cleavable thrombin linker enables peptide exchange.

13. The aAPC of claim 10, wherein the HLA class II monomer comprises DR1 fused to an Fc domain.

14. The aAPC of claim 13, wherein the Fc domain comprises an amino acid sequence comprising a cysteine substitution.

15. The aAPC of claim 14, wherein the Fc domain comprises a cysteine at position 473.

16. The aAPC of claim 10, wherein the HLA class II monomer comprises DR4 fused to an Fc domain.

17. The aAPC of claim 16, wherein the Fc domain comprises an amino acid sequence comprising a cysteine substitution.

18. The aAPC of claim 17, wherein the Fc domain comprises a cysteine at position 473.

19. An aAPC having an HLA class I molecule and an HLA class II molecule conjugated to a surface thereof.

20. The aAPC of claim 19, wherein the aAPC comprises DR1 or DR4 monomers with HLA A2-Ig dimers.

21. The aAPC of claim 19, wherein the HLA class II monomer comprises DR1 fused to an Fc domain.

22. The aAPC of claim 21, wherein the Fc domain comprises an amino acid sequence comprising a cysteine substitution.

23. The aAPC of claim 22, wherein the Fc domain comprises a cysteine at position 473.

24. The aAPC of claim 19, wherein the HLA class II monomer comprises DR4 fused to an Fc domain.

25. The aAPC of claim 24, wherein the Fc domain comprises an amino acid sequence comprising a cysteine substitution.

26. The aAPC of claim 25, wherein the Fc domain comprises a cysteine at position 473.

27. The aAPC of any one of claims 1-26, wherein the particle comprises an iron-dextran particle.

28. A method for identifying, isolating, or detecting one or more antigen-specific T cells, the method comprising:
(a) contacting a plurality of unpurified immune cells comprising one or more antigen-specific T cells with a plurality of aAPCs of any one of claims 1-27;
(b) separating antigen-specific T cells associated with the plurality of aAPCs from cells not associated with the plurality of aAPCs;
(c) recovering antigen-specific T cells associated with the plurality of aAPCs; and
(d) expanding the recovered antigen-specific T cells in culture for a period of time to provide a composition comprising antigen-specific T cells.

29. The method of claim 8, wherein the plurality of unpurified immune cells comprising one or more antigen-specific T cells are obtained from a sample comprising one or more of a peripheral blood mononuclear cell (PBMC) sample, memory T cells, naive T cells, previously activated T cells, and tumor infiltrating lymphocytes.

30. The method of claim 28 or claim 29, wherein the plurality of unpurified immune cells comprising one or more antigen-specific T cells are obtained from a sample comprising one or more of bone marrow, lymph node tissue, spleen tissue, and a tumor.

31. The method of any one of claims 28-30, wherein the plurality of unpurified immune cells are obtained from a patient or a donor.

32. The method of claim 31, wherein the donor comprises a donor who is HLA-matched to an adoptive transfer recipient.

33. The method of claim 31, wherein the plurality of unpurified immune cells are obtained from a patient and the patient has one or more diseases, disorders, or conditions selected from the group consisting of a cancer, an infectious disease, and an autoimmune disease.

34. The method of any one of claims 28-33, wherein the one or more antigen-specific T cells are selected from the group consisting of cytotoxic CD4⁺ T cells, CD4⁺ helper T cells, CD8⁺ cytotoxic T lymphocytes, T-helper 17 (Th17) cells, regulatory T cells (Tregs), and combinations thereof.

35. The method of any one of claims 28-34, wherein the expanding of the recovered cells in culture for a period of time is performed on a multi-well microtiter plate.

36. The method of claim 35, wherein the multi-well microtiter plate comprises a 96-well microtiter plate.

37. The method of any one of claims 28-36, wherein a purity of the expanded recovered antigen-specific T cells is improved relative to a method in which the antigen-specific T cells are isolated from the plurality of unpurified immune cells prior to contacting the plurality of unpurified immune cells with the plurality of aAPCs.

38. The method of any one of claims 28-37, wherein a percent of antigen-specific T cells is increased relative to a method in which the antigen-specific T cells are isolated from the plurality of unpurified immune cells prior to contacting the plurality of unpurified immune cells with the plurality of aAPCs.

39. The method of any one of claims 28-38, wherein a number of antigen-specific T cells is increased relative to a method in which the antigen-specific T cells are isolated from the plurality of unpurified immune cells prior to contacting the plurality of unpurified immune cells with the plurality of aAPCs.

40. The method of any one of claims 28-39, wherein the plurality of aAPCs comprise or consist essentially of a particle having a major histocompatibility complex class II (MHC II) molecule conjugated to a surface thereof.

41. The method of claim 40, further comprising ex vivo generation of cytotoxic CD4⁺ T cells.

42. The method of claim 40, further comprising administering a soluble costimulatory ligand to the antigen-specific T cells associated with the plurality of aAPCs after step (b).

43. The method of claim 4, wherein the costimulatory ligand is selected from the group consisting of an antibody or antigen-binding fragment thereof that specifically binds to CD28, CD80 (B7-1), CD86 (B7-2), B7- H3, 4-1BBL, 4-1BB, CD27, CD30, CD134 (OX-40L), B7h (B7RP-1), CD40, LIGHT, an antibody or antigen-binding fragment thereof that specifically binds to HVEM, an antibody or antigen-binding fragment thereof that specifically binds to CD40L, an antibody or antigen binding fragment thereof that specifically binds to OX40, and an antibody or antigen-binding fragment thereof that specifically binds to 4-1BB.

44. The method of claim 43, wherein the costimulatory ligand comprises an anti-CD28 (αCD28) antibody.

45. The method of any one of claims 28-44, further comprising administering one or more cytokines to the plurality of unpurified immune cells comprising one or more antigen-specific T cells.

46. The method of claim 45, wherein the one or more cytokines include one or more of IL-2, IL-12p70, and IFN-γ.

47. The method of any one of claims 28-36, further comprising incubating the plurality of unpurified immune cells comprising one or more antigen-specific T cells contacted with a plurality of aAPCs for a period of time at a predetermined temperature.

48. The method of claim 47, wherein the period of time is about 2 hr.

49. The method of claim 47, wherein the predetermined temperature is about 37° C.

50. The method of any one of claims 8-49, comprising a ratio of major histocompatibility complex class II (MHC II) molecule to CD4⁺ T cells is about 30 ng MHC II/10⁶ CD4⁺ T cells.

51. The method of claim 28, wherein the aAPC comprises a particle having a major histocompatibility complex class II (MHC II) molecule and major histocompatibility complex class I molecule conjugated to a surface thereof.

52. The method of claim 51, wherein the method co-activates CD4⁺ and CD8⁺ T cells.

53. The method of claim 52, wherein the co-activation of CD4⁺ and CD8⁺ T cells enhances the therapeutic function and memory formation of the CD8⁺ T cells.

54. The method of claim 52, comprising redirecting CD4⁺ T cell help of one specificity toward CD8⁺ T cells of a multitude of specificities.

55. The method of claim 28, wherein the aAPC has an HLA class I molecule and an HLA class II molecule conjugated to a surface thereof.

56. The method of claim 55, wherein the aAPC comprises DR1 or DR4 monomers with HLA A2-Ig dimers.

57. The method of claim 55, wherein the HLA class II monomer comprises DR1 fused to an Fc domain.

58. The method of claim 57, wherein the Fc domain comprises an amino acid sequence comprising a cysteine substitution.

59. The method of claim 58, wherein the Fc domain comprises a cysteine at position 473.

60. The method of claim 55, wherein the HLA class II monomer comprises DR4 fused to an Fc domain.

61. The method of claim 60, wherein the Fc domain comprises an amino acid sequence comprising a cysteine substitution.

62. The method of claim 61, wherein the Fc domain comprises a cysteine at position 473.

63. The method of claim 55, wherein the method comprises redirecting a particular HLA class II specificity to relay help from CD4⁺ T cells of that specificity to CD8⁺ T cells of a range of specificities.

64. The method of any one of claims 28-63, wherein the particle comprises a paramagnetic particle.

65. The method of claim 64, wherein the paramagnetic particle comprises an iron-dextran particle.

66. The method of claim 28, wherein the separating of the antigen-specific T cells associated with the plurality of aAPCs from the cells not associated with the plurality of aAPCs is by magnetic separation.

67. A method for treating a disease, disorder, or condition, the method comprising administering to a subject in need of treatment thereof a composition comprising one or more antigen-specific T cells prepared by the method of any one of claims 16-66.

68. The method of claim 67, wherein the disease, disorder, or condition is selected from the group consisting of a cancer, an infectious disease, and an autoimmune disease.

69. The method of claim 68, wherein the disease, disorder, or condition is a cancer and the one or more antigen-specific T cells comprise cytotoxic T cells specific for one or more tumor-associated peptide antigens to the subject in need of treatment thereof.

70. The method of claim 69, wherein the cancer comprises a solid tumor or a hematological malignancy.

71. The method of claim 70, wherein the cancer is selected from the group consisting of a melanoma, colon cancer, duodenal cancer, prostate cancer, breast cancer, ovarian cancer, ductal cancer, hepatic cancer, pancreatic cancer, renal cancer, endometrial cancer, testicular cancer, stomach cancer, dysplastic oral mucosa, polyposis, head and neck cancer, invasive oral cancer, non-small cell lung carcinoma, small-cell lung cancer, mesothelioma, transitional and squamous cell urinary carcinoma, brain cancer, a neuroblastoma, and a glioma.

72. The aAPC of any one of claims 1-27, further comprising an immunomodulatory material.

73. The aAPC of claim 72, wherein the immunomodulatory material comprises a genetic material.

74. A non-viral method for delivering an immunomodulatory material to a T cell, the method comprising administering an aAPC of claim 72 or claim 73 to a cell.
